(19) United States
(12) Patent Application Publication
(10) Pub. No.: US 2026/0118368 A1
(43) Pub. Date: Apr. 30, 2026

(54) DETECTING CANCER

(71) Applicant: UCL BUSINESS LTD, London (GB)

(72) Inventors: Samuel GAMBLE, London (GB); James READING, London (GB); Sam JANES, London (GB); Maxine TRAN, London (GB); Vitor TEIXEIRA, London (GB); Claudia PEINADOR, London (GB)

(21) Appl. No.: 19/115,597

(22) PCT Filed: Sep. 26, 2023
(86) PCT No.: PCT/EP2023/076521

§ 371 Date: Mar. 26, 2025

(30) Foreign Application Priority Data
Sep. 27, 2022 (GB) 2214114.7
Sep. 27, 2022 (GB) 2214117.0

(51) Int. Cl. G01N 33/68; C12Q 1/6869; C12Q 1/6886; G01N 15/10; G01N 15/14

(52) U.S. Cl. CPC G01N 33/6872; C12Q 1/6869; C12Q 1/6886; G01N 15/14; G01N 2015/1006; G01N 2015/1486; G01N 2015/1488; G01N 2333/705; G01N 2800/50; G01N 2800/52

(57) ABSTRACT

A method for determining whether a subject is at risk for having a progressing or high-grade pre-invasive lesion, nodule or small mass, or having a solid malignant tumour is described. The method involves analysing the proportion of T cells in a sample of blood obtained from the subject which are activated and/or exhausted T cells by analysing a trait of the T cells, e.g., via analysing biomarker expression using cytometry. In some instances, a plurality of traits is analysed and combined, e.g., via analysing biomarker expression using cytometry in combination with analysing the diversity or clonality of the blood TCR repertoire using TCR-seq.

### BACKGROUND

Cancer is a leading cause of disease worldwide. Certain types of cancer have a high chance of cure if they are detected at an early stage and adequately treated. However, many cancers are detected at a late stage and delays in cancer diagnosis can occur throughout the diagnostic pathway. This can include patients failing to recognise symptoms or delaying see a healthcare provider. Doctors may not recognise symptoms of cancers and so may not investigate them appropriately or refer on time. Furthermore, some cancers are difficult to detect. Late diagnosis is a major cause of cancer mortality.

Renal cell carcinoma (RCC) is the cause of approximately 5,000 cancer deaths in the UK per year and non-small cell lung cancer (NSCLC) causes approximately 35,000 cancer deaths in the UK per year, with a combined annual NHS cost of over £3bn. This human and financial cost is replicated around the world. More than 40% of NSCLC and 10-20% of RCC cases are diagnosed late, representing a major cause of mortality. More than 40% of patients diagnosed with non-small cell lung cancer (NSCLC) present with late-stage disease (Stage 3-4), when the cancer has spread and where 5-year survival rates are dismal (90% mortality). As a result, NSCLC is the leading cause of cancer death both worldwide and in the UK, where the disease causes over 20% of the 160,000 annual cancer deaths. In addition, NSCLC incurs significant resource burden(s) costing the NHS an estimated £2.4bn each year and a global cost estimated to exceed £150bn. Patients diagnosed with Stage 1 NSCLC are ten times more likely to survive 5 years than those diagnosed at stage 4, engendering efforts for early detection of NSCLC.

### NSCLC is Comprised of Two Major Histological Subtypes;

i) lung adenocarcinoma (LUAD), which is localised to the lung parenchyma in the periphery and accounts for approximately 60-65% of disease and
ii) lung squamous cell carcinoma (LUSC) is found in the central airways, which constitutes approximately 30% of disease.

Each subtype has distinct forms of pre-malignant disease due to unique clinical and genomic features meaning that LUAD and LUSC are preferentially detected via specific screening approaches.

Low-dose computerised tomography (LDCT) lung screening is advocated as a major pathway for early lung cancer detection and predominantly detects LUAD. This strategy increases early diagnosis and reduces mortality by 20-26% but even the perfectly executed CT screening programme will only prevent 20% of deaths, as it targets the highest-risk patients, missing lighter and never smokers. Additional disadvantages of LDCT include resource intensiveness, radiation exposure and failure to detect most LUSCs. LDCT also detects pre-malignant and benign peripheral lung nodules, but these have indeterminate disease penetrance and require continual surveillance.

The pre-invasive stages of LUSC (‘pre-LUSC’) are characterised by asymptomatic lesions that develop in a stepwise process of increasing dysplasia and can be classified as low-grade (LG) and high-grade (HG). LG lesions are associated with no increased risk of NSCLC but 40-90% of patients with HG lesions progress. Traditional screening of pre-LUSC relies on sputum cytology which has poor sensitivity, and autofluorescence bronchoscopy which suffers from high false-positive rates, low patient throughput and the burden(s) associated with an invasive procedure. Similarly, whilst a 280-gene classifier of airway bronchial cells has been successfully used to predict the presence of pre-LUSC lesions this remains an expensive and invasive approach.

New non-invasive, tools to detect progressing lesions/nodules would address a vast clinical unmet need. Circulating tumour DNA (ctDNA) is the most widely proposed future clinical strategy for liquid multi-cancer detection but detects only 40-50% of stage I-II NSCLC cancers, falling to as low as 10% for stage I LUAD and is expected to be less effective in the pre-invasive setting.

Thus, current NSCLC detection protocols are often insensitive, non-specific, time-consuming, resource-heavy, invasive/painful and can lead to anxiety and over diagnosis or delayed diagnosis.

It is known that T cell differentiation is skewed in subjects with established or late stage cancer. The balance of T cell populations in a subject shifts towards the majority of T cells exhibiting biomarkers that are associated with having identified their cognate antigen on a tumour. This has mostly been described as a process within the tumour or tissue, with sparse evidence that this could also be seen in the blood.

However, there is still no means for early cancer detection at the stage of detecting a progressing or high-grade pre-invasive lesion or nodule or solid tumours before the cancer has fully developed in the subject.

There therefore remains an unmet and urgent need for early detection of NSCLC and other solid cancers. In particular, a new blood test with high specificity and sensitivity could fulfil this need as a non-invasive classifier of progressing preinvasive disease or early established disease.

The ability to distinguish between subjects with early-stage solid cancers or pre-invasive lesions or nodules likely to progress to cancer, versus subjects with no malignancy or with lesions or nodules unlikely to progress would enable early, curative intervention or targeted surveillance, saving lives and significantly reducing resource burden.

### The Present Invention

The present invention aims to solve these and other problems by providing a novel method for determining whether a subject is at risk for having a progressing or high-grade pre-invasive lesion or nodule or a solid malignant tumour of any stage. The present invention provides, for the first time, a non-invasive immune-based method for distinguishing between low-risk pre-invasive lesions or nodules, low grade pre-invasive lesions or nodules (or lack of tumour), and progressing pre-invasive lesions or nodules, high grade pre-invasive lesions or nodules (or having a solid tumour, e.g., a stage I solid tumour). The present invention also detects pre-invasive lesions or nodules (e.g., high grade pre-invasive lesions or nodules) that are at risk of progressing via measuring immunological markers in the blood, and provides a readout as compared with a healthy subject or a plurality of healthy subjects.

The use of a combination multi-omic biomarkers to determine a subject's T cell differentiation state in a blood sample obtained from a subject, that can be used in the early detection setting to detect the presence of progressive pre-invasive lesions or nodules, or early-stage tumours (e.g., stage I tumours) is described herein for the first time.

When T cells recognise non-self antigen they differentiate from a naïve or resting state into a range of activated, exhausted and memory T cells. These changes can be measured by cytometry. Cytometry in this context, is used to profile the frequency and intensity of biomarkers on T cells. The biomarkers to be detected can be those related to T cell activation, exhaustion and memory differentiation. Combinations of these biomarkers can be used to determine the strength or type of immune activation in disease states such as infection, autoimmunity, transplantation and cancer. These biomarkers can be measured within CD3+ live cells in the blood (viable T cells) and amongst the major T cell lineages of killer cytotoxic T cells (‘CD8+ T cells’) or helper & regulatory (‘CD4 T cells’). Other methods such as TCR-seq are also disclosed herein which can also be used to interrogate the immune landscape and track T cell traits before neoplasia actually develops. The present invention therefore demonstrates multiple ways to track T cell traits and predict progression into cancer by measurements made in the periphery using only blood. The combined use of these tools can also be used to more significantly differentiate between patient groups at risk of developing or with malignant disease as an early cancer detection tool to detect pre-invasive neoplasia.

In chronic infections like HIV-1 and HCV there is well established expansion of activated, memory and exhausted T cells at the expense of resting and naïve T cells in the blood. The level of activated and exhausted T cells reflects the presence of disease compared to healthy individuals and the amount of virus detectable. This shift in differentiation is termed T cell differentiation skewing. It has been discovered that a similar remodelling of T cell differentiation inside the tumours of patients with established NSCLC and RCC; is characterised by T cells co-expressing biomarkers of exhaustion activation and terminal differentiation and loss of stem-like populations expressing biomarkers of early-differentiation and progenitor potential.

T cell differentiation skewing in the blood of cancer patients is less well characterised.

The present inventors have shown in NSCLC, that (neo) antigen load (inferred by tumour mutational burden) correlated with T cell differentiation skewing inside the tumour (loss in resting, progenitor and naïve cells, increase in terminally differentiated, exhausted and activated T cells).

The use of T cell differentiation skewing, defined as a loss of resting/naïve T cells and a gain in activated/exhausted T cells as a method of early cancer detection, such as early lung cancer detection, has not previously been described. Prior to the present invention there is no published data to show that the presence of pre-invasive LUSC lesions or pre-LUAD nodules are associated with blood T cell differentiation skewing. Equally prior to the present invention there was no data to support an equivalent process in another solid tumour type.

Prior to the present invention, existing methods and strategies, such as disclosed in US2018356420 A1 have focused on using antibody panels to analyse T cells within patients with established cancer. However, these methods have limitations as they cannot be used for distinguishing between progressing and non-progressing lesions or nodules, nor for determining whether a subject is at risk for having a progressing or high-grade pre-invasive lesion or nodule or having a very early stage (stage 1) solid tumour.

Elsewhere in earlier studies, Li et al, Lung Cancer 162 (2021) 16-22 describes lung cancer-associated T cell repertoire as potential biomarker for detection of stage lung cancer. The authors do not propose detection of lesions or nodules before a cancer is established. The authors do not propose analysis of the T cell repertoire for distinguishing between progressing and non-progressing lesions or nodules, nor for determining whether a subject is at risk for having a progressing or high-grade pre-invasive lesion or nodule. The authors do not use the ratio of exhausted/activated to naïve/resting T cells as a method for early stage cancer detection.

Mascaux et al, 570, Nature, Vol 571, 25 Jul. 2019, relates to immune evasion before tumour invasion in early lung squamous carcinogenesis and indicates that the adaptive immune response within tumours may be strongest at the earliest stage of carcinoma. However, there is no suggestion of distinguishing between progressing and non-progressing lesions or nodules, nor determining whether a subject is at risk for having a progressing or high-grade pre-invasive lesion or nodule or having a solid tumour.

WO2021188941 A1 relates to methods for isolating T cells and T-cells receptors from peripheral blood by single-cell analysis for immunotherapy, in addition to preparing and enriching a population of T cells having antigenic specificity for a target antigen. However, the authors focused on analysing and using the T cell repertoire as a therapeutic strategy for established cancer in patients and does not propose a method for distinguishing between progressing and non-progressing lesions or nodules, nor for determining whether a subject is at risk for having a progressing or high-grade pre-invasive lesion or nodule or having a solid tumour.

Cancers, vol. 14, 2022, “Martinez-Gomez et al” identifies certain T cell biomarkers as markers for tumor specific T cells or T cells which are a surrogate for an active immune response. However, this work is focused on samples with established disease, with no suggestion or teaching of what biomarkers, if any, could be used for pre-invasive or early stage (e.g., stage) disease.

Immune Network, vol 20(6), 2020, “Kim et al” article e48, similarly focuses on T cells in cancer patients, and more specifically highlights the difference between those that do or do not respond to checkpoint inhibitors with clinical benefit. However, this document also only relates to later stage disease and response to a therapy. This does not show any patients with preinvasive or early-stage disease who are distinguished by these cells compared with no disease.

WO 2016/185182 describes methods of assessing whether an individual has an exhausted CD8+ T cell or lack of CD4+ T cell co-stimulation phenotype to determine an individual's risk of autoimmune disease progression, progression of a chronic infection or cancer progression. In contrast, the present invention relates to measuring T cell differentiation for cancer early detection (i.e., at a pre-invasive stage, or early stage (e.g., stage I), as opposed to forecasting progression of patients with established disease. There is no suggestion to combine different T-cell analyses, for example, using cytometry and TCR-seq, let alone as a tool for the early detection of cancer (e.g., at an early or the pre-invasive stage).

Clinical Cancer Research, vol 27, 2021, Laumont et al focuses on the prognostic potential of exhausted T cells infiltrating established ovarian cancer. However, this article does not allude to measuring phenotypic and/or TCR-seq features in the blood of individuals at risk of developing or having progressive pre-invasive or early-stage disease for the purposes of early cancer detection. This article also does not describe combining phenotypic and TCR-seq features in the blood.

Journal of Pathology, vol. 251, 2020, Guo et al, pp 26-27 references using TCR-seq and transcriptional measures of T cells in the blood of patients with renal cell carcinoma. However, this document does not teach nor suggest what traits can be used to forecast cancer development (e.g., at a pre-invasive stage), nor does this document teach how to discriminate between progressive (e.g., high-grade) vs low grade pre-invasive neoplasia. This document also fails to teach of a method that can be used for early detection cancer screening via a non-invasive blood test. Finally, this document does not disclose that multiple methods (e.g., cytometry and TCR-seq) can be combined to forecast cancer development using a multiomic model.

### STATEMENTS OF THE INVENTION

The present invention provides a method for determining whether a subject is at risk for having a progressing or high-grade pre-invasive lesion, nodule or small mass, or having a solid malignant tumour, the method comprising analysing the proportion of T cells in a sample of blood obtained from the subject which are activated and/or exhausted T cells by analysing a trait of the T cells. In some embodiments, the solid malignant tumour is a stage I malignant tumour.

The present invention also provides a method for determining whether a subject is at risk for having a progressing or high-grade pre-invasive lesion, nodule or small mass, the method comprising analysing the proportion of T cells in a sample of blood obtained from the subject which are activated and/or exhausted T cells by analysing a trait of the T cells.

Analysing the proportion of T cells in the sample of blood obtained from the subject which are activated and/or exhausted may comprise analysing a plurality of traits of the T cells.

The method may further comprise combining results obtained from analysing the plurality of traits, preferably wherein the traits are (i) the diversity and/or clonality of T cell receptors as determined by TCR Seq and (ii) biomarkers expressed by T cells as determined by flow cytometry.

The trait may be a phenotypic trait.

The phenotypic trait may be the diversity (i.e., diversity or clonality) of T cell receptors on the T cells in the sample of blood, optionally the diversity (i.e., diversity or clonality) of CDR3B on TCRs of the T cells in the sample of blood, further optionally using TCR Seq. The phenotypic trait may be the diversity or clonality of T cell receptors on the T cells in the sample of blood, optionally the diversity or clonality of CDR3B on TCRs of the T cells in the sample of blood, further optionally using TCR Seq.

In some embodiments, the phenotypic trait is biomarker expression by the T cells in the sample of blood using cytometry, preferably flow cytometry.

The present invention also provides a method for determining whether a subject is at risk for having a progressing or high-grade pre-invasive lesion, nodule or small mass, or having a solid malignant tumour, the method comprising analysing the proportion of T cells in a sample of blood obtained from the subject which are activated and/or exhausted T cells by analysing a trait of the T cells, wherein the trait is a phenotypic trait which is: a) the diversity or clonality of T cell receptors on the T cells in the sample of blood, and/or b) biomarker expression by the T cells in the sample of blood, optionally using cytometry. In some embodiments, the method comprises analysing a plurality of traits of the T cells, and the method comprises combining results obtained from analysing the plurality of traits of the T cells, which involves combining results obtained from a) and b)

The present invention also provides a method for determining whether a subject is at risk for having a progressing or high-grade pre-invasive lesion, nodule or small mass, the method comprising analysing the proportion of T cells in a sample of blood obtained from the subject which are activated and/or exhausted T cells by analysing a trait of the T cells, wherein the trait is a phenotypic trait which is
a) the diversity or clonality of T cell receptors on the T cells in the sample of blood and/or
b) biomarker expression by the T cells in the sample of blood, optionally using cytometry. In some embodiments, the method comprises analysing a plurality of traits of the T cells, and the method comprises combining results obtained from analysing the plurality of traits of the T cells, which involves combining results obtained from a) and b).

The present invention also provides a method for determining whether a subject is at risk for having a progressing or high-grade pre-invasive lesion, nodule or small mass, or having a solid malignant tumour, using a sample of blood obtained from the subject, the method combining two or more T cell analyses comprising a) measuring the ratio of activated and/or exhausted T cells:naïve and/or resting T cells using cytometry, or measuring the proportion of activated and/or exhausted cells as a percentage of T cells using cytometry and
b) the diversity or clonality of the blood TCR repertoire.

In embodiments of the above methods, the present invention also comprises analysing a plurality of traits of T cells in a sample of blood obtained from the subject. The phenotypic traits (or diversity or clonality) can be diversity of T cell receptors, which may be analysed, for example, by TCR Seq, and biomarkers expressed by T cells, which may be analysed, for example, by flow cytometry.

For the above methods, in some embodiments, the subject is at risk for having, or has, a progressing or high-grade pre-invasive lesion, nodule or small mass; in some embodiments, the subject is at risk for having, or has, a solid malignant tumour, e.g., a stage I solid malignant tumour.

### For all the Above Methods, the Subject May be at Risk if:

a) the diversity of the repertoire of TCRs is lower than the diversity of the repertoire of TCRs of a comparison subject or a plurality of comparison subjects,
b) D50 diversity score is lower than a D50 diversity score obtained from analysing T cells in a sample of blood obtained from a comparison subject or a plurality of comparison subjects,
c) a clonality score is higher than a clonality score obtained from analysing T cells in a sample of blood obtained from a comparison subject or a plurality of comparison subjects, or
d) Hill number is lower than a Hill number obtained from analysing T cells in a sample of blood obtained from a comparison subject or a plurality of comparison subjects.

### The Subject May be at Risk if:

(e) the clonality of the repertoire of TCRs occupied by the top n clones is higher than the clonality of the repertoire of TCRs occupied by the top n clones in a comparison subject or a plurality of comparison subjects, optionally wherein n is between 50 and 1000, preferably wherein n=100
(f) the proportion of the repertoire of TCRs occupied by small clones is lower than the proportion of the repertoire of TCRs occupied by small clones in a comparison subject or a plurality of comparison subjects, or
(g) the proportion of the repertoire of TCRs occupied by large clones is higher than the proportion of the repertoire of TCRs occupied by large clones in a comparison subject or plurality of comparison subjects or
(h) the ratio of the repertoire of TCRs occupied by large clones:the repertoire of TCRs occupied by small clones is higher than the ratio of the repertoire of TCRs occupied by large clones:the repertoire of TCRs occupied by small clones in a comparison subject or a plurality of comparison subjects

The comparison subject or the plurality of comparison subjects is/are healthy subject(s) or a part of the general population.

In some embodiments, the phenotypic trait is biomarker expression by the T cells in the sample of blood using cytometry, preferably spectral or flow cytometry.

The phenotypic trait may be biomarker expression by the T cells in the sample of blood, optionally using cytometry, further optionally wherein cytometry is used to detect the presence or absence of a panel of biomarkers comprising Ki67 and CD39.

In some embodiments, the activated and/or exhausted T cells express Ki67 and CD39. The panel of biomarkers may further comprise one or more biomarkers selected from CD45RA, CCR7, PD-1, CD57 or CD38, or FoxP3.

The panel of biomarkers may further comprise one or more biomarkers selected from CD3, CD4 or CD8. In some embodiments, the T cells are CD3+ T cells (i.e., the T cells express the CD3 biomarker). In some embodiments, the T cells are CD4+ T cells (i.e., the T cells express the CD4 biomarker) or CD8+ T cells (i.e., the T cells express the CD8 biomarker). In some embodiments, the T cells are CD3+CD4+ T cells (i.e., the T cells express the CD3 and CD4 biomarker) or CD3+CD8+ T cells (i.e., the T cells express the CD3 and CD8 biomarker). The analysis may comprise use of a viability dye (e.g., to determine live CD3+ T cells).

The panel of biomarkers may further comprise CD45RA, or CCR7, or CD45RA and CCR7, or FoxP3.

The panel of biomarkers may further comprise CD45RA, CCR7, and PD-1. or CD45RA, CCR7, PD-1 and FoxP3

The panel of biomarkers may further comprise CD45RA, CCR7, PD-1 and CD57, or CD45RA, CCR7, PD-1 and CD57 and FoxP3.

The panel of biomarkers may further comprise CD45RA, CCR7, PD-1, CD57 and CD38, or CD45RA, CCR7, PD-1, CD57, CD38 and FoxP3

The panel of biomarkers may further comprise CD45RA, CCR7, CD57, and CD38, or CD45RA, CCR7, CD57, CD38 and FoxP3

The panel of biomarkers may further comprise CD45RA, PD-1 and CD57, or CD45RA, PD-1, CD57 and FoxP3.

In some embodiments, the activated and/or exhausted T cells express Ki67 and CD39, wherein the T cells are CD4 T cells or CD4+CD3+ T cells. In some embodiments, the activated and/or exhausted T cells express Ki67 and CD39, wherein the T cells are CD8+ T cells or CD8+CD3+ T cells. In some embodiments, the activated and/or exhausted T cells express Ki67 and CD39, and do not express CD45RA−, CCR7 and PD1. In some embodiments the T cells are CD8+ T cells or CD3+CD8+ T cells.

In some embodiments, cytometry is used to detect the presence of a panel of biomarkers comprising FoxP3 and CD4, preferably flow cytometry. In some embodiments, the panel of biomarkers may further comprise one or more biomarkers selected from CD45RA, CCR7, PD-1, CD57 Ki67, or CD39. In some embodiments, the panel of biomarkers may further comprise CD3. In some embodiments, the analysis may comprise use of a viability dye.

In some embodiments, the activated and/or exhausted T cells express FoxP3, wherein the T cells are CD4 T cells. In some embodiments the T cells are CD3+CD4+ T cells. In some embodiments, the activated and/or exhausted T cells express FoxP3 and CD39, optionally wherein the activated and/or exhausted T cells further (i) express the biomarker Ki67 or (ii) do not express the biomarker CD45RA.

In some embodiments, the activated and/or exhausted T cells express the biomarkers Ki67 and CD39.

### The Subject May be at Risk if:

i) the ratio of activated and/or exhausted T cells:naïve and/or resting T cells is equal to or greater than
a ratio of activated and/or exhausted T cells:naïve and/or resting T cells of a comparison subject, or
an average ratio of activated and/or exhausted T cells:naïve and/or resting T cells of a plurality of comparison subjects, or
ii) the ratio of activated and/or exhausted T cells:T cells which are not activated and/or exhausted is equal to or greater than
a ratio of activated and/or exhausted T cells:T cells which are not activated and/or exhausted of a comparison subject, or
an average ratio of activated and/or exhausted T cells:T cells which are not activated and/or exhausted of a plurality of comparison subjects, or
iii) the proportion of activated and/or exhausted T cells as a percentage of T cells is higher than the proportion of activated and/or exhausted T cells in a comparison subject or a plurality of comparison subjects, optionally wherein the activated and/or exhausted T cells are CD4+ regulatory T cells (Tregs) (i.e., the activated and/or exhausted T cells are CD4+ T cells which express FoxP3) or effector regulatory T cells.
v) the proportion of naïve T cells as a percentage of T cells is lower than the proportion of naïve T cells as a percentage of T cells in a comparison subject or a plurality of comparison subjects, optionally wherein the naïve cells are naïve CD4+ T cells.
   A Comparison Subject May be Selected from:
a) a subject known to have a progressing or high-grade pre-invasive lesion, nodule or small mass or an established solid malignant tumour,
b) the subject at a different time point.
   A Plurality of Comparison Subjects May be Selected from:
a) a plurality of subjects known to have a progressing or high-grade pre-invasive lesion, nodule or small mass or an established solid malignant tumour,
b) a plurality of healthy subjects, or
c) a plurality of subjects of the general population.

For all above embodiments, the cytometry may comprise one or more of flow cytometry, spectral cytometry or mass cytometry, optionally the cytometry comprises flow cytometry.

The present invention also provides for a method for determining whether a subject is at risk for having a progressing or high-grade pre-invasive lesion, nodule or small mass, or having a solid malignant tumour, using a sample of blood obtained from the subject, the method combining two or more T cell analyses comprising:
a) measuring the ratio of activated and/or exhausted T cells:naïve and/or resting T cells determined by flow cytometry, or measuring the proportion of activated and/or exhausted cells as a percentage of T cells determined by flow cytometry and
b) the diversity or clonality of the blood TCR repertoire. In some embodiments, the activated and/or exhausted T cells express the biomarkers Ki67 and CD39. In some embodiments, the activated and/or exhausted T cells express the biomarker FoxP3, wherein the T cells are CD4+ T cells, (optionally in combination with CD39, and further optionally (i) in combination with CD39 and Ki67, or (ii) in combination with CD39 and in the absence of CD45RA). The diversity and clonality or the blood can be measured by any suitable technique. In some embodiments and examples, the diversity or clonality of the blood is measured using the D50 index. In some embodiments and examples, the diversity or clonality of the blood is measured using the ratio of the repertoire of TCRs occupied by large clones:the repertoire of TCRs occupied by small clones.

The present invention also provides a method of treating a subject determined to be at risk for having a progressing or high-grade pre-invasive lesion, nodule or small mass, or having a solid malignant tumour, comprising analysing the proportion of T cells in a sample of blood obtained from the subject which are activated and/or exhausted T cells by analysing a trait of the T cells, wherein the trait is a phenotypic trait wherein the analysing comprises
a) analysing the proportion of T cells in a sample of blood obtained from the subject which are activated and/or exhausted T cells using biomarker expression, optionally using cytometry, wherein the subject is at risk if
(i) the ratio of activated and/or exhausted T cells:naïve and/or resting T cells is equal to or greater than a ratio of activated and/or exhausted T cells:naïve and/or resting T cells of a comparison subject, or in a plurality of comparison subjects, or
(ii) the ratio of activated and/or exhausted T cells:T cells which are not activated and/or exhausted is equal to or greater than a ratio of activated and/or exhausted T cells:T cells which are not activated and/or exhausted of a comparison subject, or in a plurality of comparison subjects.
(iii) the proportion of activated and/or exhausted T cells is higher than the proportion of activated and/or exhausted T cells in a comparison subject or a plurality of comparison subjects, and/or
b) determining the diversity or clonality of T cells in a sample of blood obtained from the subject, optionally using TCR-seq, wherein the subject is at risk if
(i) the diversity of the repertoire of TCRs is lower than the diversity of the repertoire of TCRs of a comparison subject or a plurality of comparison subjects,
ii) D50 diversity score is lower than a D50 diversity score obtained from analysing T cells in a sample of blood obtained from a comparison subject or a plurality of comparison subjects,
iii) a clonality score is higher than a clonality score obtained from analysing T cells in a sample of blood obtained from a comparison subject or a plurality of comparison subjects, or
iv) the Hill number is lower than a Hill number obtained from analysing T cells in a sample of blood obtained from a comparison subject or a plurality of comparison subjects, or
v) the clonality of the repertoire of TCRs occupied by the top n clones is higher than the clonality of the repertoire of TCRs occupied by the top n clones in a comparison subject or a plurality of comparison subjects, optionally wherein n is from 50-1000, and preferably wherein n=100, or
vi) the proportion of the repertoire of TCRs occupied by small clones is lower than the proportion of the repertoire of TCRs occupied by small clones in a comparison subject or a plurality of comparison subjects, or
vii) the proportion of the repertoire of TCRs occupied by large clones is higher than the proportion of the repertoire of TCRs occupied by large clones in a comparison subject or a plurality of comparison subjects or
viii) the ratio of the repertoire of TCRs occupied by large clones:small clones is higher than the ratio of the repertoire of TCRs occupied by large clones:small clones in a comparison subject or a plurality of comparison subjects, optionally wherein the comparison subject is a healthy subject, or the plurality of comparison subjects are healthy subjects, and
treating the subject if the subject is at risk.

In some embodiments, the treating may comprise administering an anti-cancer therapeutic. In some embodiments, the treating may comprise administering a therapeutic suitable for treating pre-invasive neoplasia and/or a high grade pre-invasive lesion, nodule or small mass. In some embodiments, the treating may comprise administering a therapeutic suitable for treating a solid malignant tumour, for example, a stage I solid malignant tumour. In some embodiments, the treating may comprise electrocautery, argon plasma coagulation (APC), cryotherapy and photodynamic therapy (PDT). The latter are all minimally invasive treatment options that may be used for the treatment of high-grade pre-invasive lesions, as described in Daniels et al; Ther. Adv. Med Oncol. 2013 July; 5(4); 235-248.

The novelty and inventiveness of the present disclosure does not rely on the techniques to identify T-cell traits themselves (e.g., using flow cytometry and/or TCR-seq, since these are well-known techniques), but the use of these techniques to analyse the peripheral blood from pre-invasive cancer patients, e.g., pre-invasive lung cancer patients. Most of the research is carried out in early-stage invasive disease, highlighting the rarity of the cohort used. These samples have been used to interrogate the immune landscape even before invasive neoplasia actually develops. The present invention demonstrates multiple ways to track T cell traits and predict progression into cancer in the periphery using only blood. While prior research has also looked at the presence of activated/exhausted T cells and has used TCR clonality to identify tumour-reactive T cells, such techniques have only previously been demonstrated in established disease. For the first time, the present inventors have uniquely demonstrated that these tools can be used for the early detection of cancer in the context of pre-invasive disease (e.g., a high grade pre-invasive lesion, nodule or small mass, or pre-invasive neoplasia).

Further, the present inventors have demonstrated that the combined use of flow cytometry and TCR-seq can be used as an early detection tool for cancer. These techniques capture different parts of the peripheral biology and differentiation skewing occurring in pre-invasive patients, therefore providing a synergistic effect. Prior methods have not used a combination of these techniques, let alone in the context of pre-invasive disease.

In some examples, the term “comprising analysing the proportion of T cells in a sample of blood obtained from the subject which are activated and/or exhausted T cells by analysing a trait of the T cells” may encompass or be used interchangeably with the term “determining a subject's T cell differentiation state in a blood sample”. Therefore, also disclosed herein is a method for determining whether a subject is at risk for having a progressing or high-grade pre-invasive lesion, nodule or small mass, or having a solid malignant tumour, the method comprising determining a subject's T cell differentiation state in a blood sample obtained from a subject. In some embodiments, a subject's T cell differentiation state is determined (i) using cytometry (and optionally flow cytometry) to detect the presence or absence of a panel of biomarkers and/or (ii) TCR-seq to determine the diversity or clonality of the blood TCR repertoire. In some embodiments, the solid malignant tumour is a stage I malignant tumour.

In some examples, the term “high-grade pre-invasive lesion, nodule or small mass” may encompass or be used interchangeably with the term “pre-invasive neoplasia”, “pre-invasive neoplastic lesion”, as well as other terms described elsewhere herein.

For any method defined herein describing a solid malignant tumour, the solid malignant tumour is a stage I solid malignant tumour (i.e., an early-stage solid malignant tumour). This is distinguished from more established tumours in later stages.

### BRIEF DESCRIPTION OF THE DRAWINGS

For a better understanding of the present invention, and to show more clearly how it may be carried into effect, reference will now be made, by way of example, to the accompanying drawings, in which:

FIG. 1 shows the use of biomarkers (CD39, Ki67) to classify low from high grade pre-LUSC. The data uses a combination of CD4 and CD8 T cells. In this example the data is shown as a generated TEDI score (short hand for the T cell early detection index, developed by the inventors) that shows the capacity to classify low from high-grade pre-LUSC. A) Workflow for how to generate the TEDI score, B) result of non parametric Mann-Whitney, One-tailed test of the Minimal. TEDI score between low-grade and high-grade pre-LUSC patients as described above. C) Gating strategy for identifying cell populations required to generate the score and D) the associated calculation.

FIG. 2 shows a workflow showing the protocol of how the computational high dimensional analysis and clustering of the flow cytometry data was completed following the analysis steps outlined. The Figure shows each major step in how a skilled operator should analyse files from a flow cytometer using the R programming environment (https://www.r-project.org/) to obtain a list of all T cell clusters within a set of samples, and the proportion of each cluster in each sample, including the exhausted/activated and resting/naïve T cell clusters used in the invention. All packages described are freely available to download at https://cran.r-project.org/or https://www.bioconductor.org/. The person skilled in the art will know that the workflow provided is merely an example in the context of the Examples provided herein and is not intended to limit the invention in any way.

FIG. 3 shows that systemic T cell differentiation skewing distinguishes patients with high vs low-grade pre-invasive central airway lesions. A) Flow cytometry data from PBMCs showing Uniform Manifold Approximation Projections (UMAPs) of CD4 (left) and CD8 (right) viable T cells from 66 samples (31 high vs 35 low grade) of 30 patients (14 high vs 16 low Grade) with pre-invasive lesions. This shows which clusters of T cells are present across all samples and their relative abundance. Individual clusters and major subsets of cells are indicated. B) Heatmaps of biomarker expression in each cluster for CD4 and CD8 T cells. This shows that there are 31 different clusters T cell present in the blood of patients with pre-LUSC, and details which biomarkers describe those different clusters. Clusters are in rows and biomarkers are in columns. For each cluster the drawing shows whether a biomarker is highly, lowly, or intermediately expressed. The lines on the left are a dendrogram, which shows how similar clusters of T cells are related. All clusters within CD8 T cells are on the left heatmap, and those within CD4 T cells are on the right. This shows that there are multiple types of activated/exhausted and resting/naïve T cell present in the pool of samples. C) Volcano plots showing significant differences in cluster frequency for CD8 (left) and CD4 (right) T cells. Clusters enriched in high-grade disease are shown on the left of each plot and those enriched in low grade disease on the right. Samples from patients that progressed or regressed between lesion grades were excluded for analysis. This shows which clusters of T cell are increased in high-grade disease, and which are increased in low grade disease. Activated/exhausted T cells are increased in high-grade and resting/naïve are increased in low grade disease. D) The ratio of clusters enriched in high vs low grade (activated/exhausted vs resting progenitor) was developed as a potential classifier and plotted per patient for CD8 (left), CD4 (centre) or all T cell subsets (right). This shows that the ratio of activated/exhausted:naïve/resting T cells is significantly higher in high vs low grade disease. E) Receiver operator characteristic curves (ROCs) for the ratio of activated/exhausted:naïve/resting T cells within CD4 and CD8 T cells, labelled as CD4 TEDI and CD8 TEDI, respectively. This shows that 92-94% of patients can be correctly classified as High or Low-grade using the invention. P values from one-tailed, unpaired Wilcoxon test. E) ROC curves of the metrics from flow cytometry and data indicated, AUC, Area Under Curve. TEDI, T cell early detection index.

FIG. 4 shows CD4 T cell differentiation skewing in pre-invasive lung neoplasia. A) Summary of biomarker expression in clusters that show significantly different abundance in high vs low grade disease according to sample level analysis. The mean frequency of each cluster was calculated per patient and shown in B) volcano plot and C) bar plots. D) Cluster names, median biomarker expression and annotation of enrichment in high-grade or low-grade. This Figure shows which clusters of T cell are present amongst CD4 T cells and highlights those that are significantly different in high or low-grade disease in detail, indicating expression of key biomarkers CD39 and Ki67 and additional biomarkers PD1, CCR7, CD45RA, CD57 and CD38.

FIG. 5 shows CD8 T cell differentiation skewing in pre-invasive lung neoplasia. A) Summary of biomarker expression in clusters that show significantly different abundance in high vs low grade disease according to sample level analysis. The mean frequency of each cluster was calculated per patient and shown in B) volcano plot and c) bar plots. D) Cluster names, median biomarker expression and annotation of enrichment in high-grade or low-grade. This Figure shows which clusters of T cell are present amongst CD8 T cells and highlights those that are significantly different in high or low-grade disease in detail, indicating expression of key biomarkers CD39 and Ki67 and additional biomarkers PD1, CCR7, CD45RA, CD57 and CD38.

FIG. 6 shows a basic workflow showing how to take a blood sample and measure the change in CD4 or CD8 clusters as a single score referred to as the T cell early detection index (TEDI). To generate TEDI, a ratio of [sum freq. of all CD4 T cell clusters enriched in HG disease]/[sum freq. of all clusters enriched in LG disease] was calculated and the process was repeated for CD8 T cells. Activated/exhausted T cells were increased in high-grade and resting/naïve are increased in low-grade disease.

This is a key step in using the invention. The person skilled in the art will know that the workflow provided is merely an example in the context of the Examples provided herein and is not intended to limit the invention in any way.

FIG. 7 shows T cell early detection indices (TEDI) in high vs low-grade disease. Clusters associated with high-grade disease were summed and divided by the sum frequency of clusters associated with low grade disease to generate a single score referred to as the TEDI. This was calculated per patient according to clusters in CD4 or CD8 T cells or the average of both (combined). Each dot is a patient, p values from one way Wilcoxon tests, dotted lines represent the sensitivity adjusted cut-off values in the table below. This drawing summarises the key results from the example, showing that the ratio of exhausted/activated:naïve resting T cells is significantly higher in high vs low-grade disease. The person skilled in the art will know that the use of a TEDI provided is an in-house term that is specific to the inventors and that various methods and techniques discussed herein can be performed using a variety of different apparatus, assay conditions, computational hardware and software components that may result in different numerical values obtained, but which equate to the same biological results to the Examples provided herein. Therefore, the use of a TEDI is merely an example in the context of the Examples provided herein and is not intended to limit the invention in any way.

FIG. 8 shows the gating strategy for manual identification of high and low grade associated T cell populations. Gating (from left to right upper panel) shows identification of live, single, CD3+ lymphocytes, within live T cells CD4 and CD8 T cells were identified and then gating of key CD8 and CD4 T cell subsets that were enriched in high-grade samples and low-grade samples. This drawing shows how a skilled researcher could bypass computational analysis to identify several activated/exhausted and naïve/resting T cell clusters manually using compensated FCS (Flow Cytometry Standard) files in FlowJo software.

FIG. 9 shows data from RCC patients showing an increase in systemic T cell differentiation in malignant (n=16 samples from 10 patients) vs benign (n=3 samples from 3 patients) disease. A) UMAP of FlowSOM defined T cell clusters from PBMC of all samples stained with 31 biomarkers and analysed by spectral cytometry. 5000 live CD3+ events per samples were down-sampled for analysis. B) The ratio of progenitor vs exhausted CD4 (left), CD8 (centre) or combined (right) T cell subsets. P values from one-tailed, unpaired Wilcoxon test. This drawing shows that the ratio of activated/exhausted:naïve/resting T cells is increased in the blood of patients with renal cancer vs patients with benign disease.

FIG. 10 shows TCRseq data from pulmonary pre-invasive neoplasia. A-B) TCRseq analysis on PBMCs from 22 patients. Each sample is a different patient. Proportion of the repertoire occupied by clonotypes of the indicated size was analysed and B) compared between high vs low grade disease. C-E) TCRseq metrics in high- and low-grade patients. Boxplots display C) D50 diversity index, D) clonality score, and E) Hill number Significance determined by unpaired, one-tailed Wilcoxon test. The Figure indicates that there is a significant difference in some of these TCRseq metrics between high- and low-grade patients, possibly reflective of persistent TCR engagement from a chronic antigen burden. D50 (AUC=88.4%), Hill number (AUC=86%).

FIG. 11 shows an ROC curve for significant TCRseq metrics identified from FIG. 10. ROC curves for the TCRseq metrics listed for all analysed TCRseq PBMCs in 22 patients. Hill number AUC 86%, D50 AUC 88.4%. The Figure shows that as the D50 has the most significant difference between high- and low-grade disease (FIG. 10C) and has the greatest diagnostic power (FIG. 11), this metric has the highest potential.

FIG. 12 shows a multi-omic approach and the combining of data from a plurality of T cell analyses. FIG. 12 shows ROC curves of the metrics from flow cytometry and TCRseq data, in addition to results using a combination of these techniques. (A) AUC values and ROC analysis from important flow cytometry (CD4 and CD8 TEDI) and TCRseq analysis used alone. (B) The relative importance of multiple metrics from flow cytometry and TCRseq assays (‘feature importance’) as determined by the XGBoost machine learning library. This is a combined methodology used multiple metrics to classify patients with high vs low grade disease and shows that TEDI CD4 from flow cytometry in addition to the D50 index from TCRseq are important classifiers within a combined multivariate classifier. The combined methodology showed improved results (C) for classifying patients with high vs low grade disease compared to the use of cytometry and TCRseq alone, as shown by ROC analysis. This Figure demonstrates that both cytometry and TCRseq approaches can be used to effectively distinguish subjects into high-grade and low-grade disease. These techniques can also be combined together by incorporating multiple metrics from these techniques into a single analysis, to generate data that further improves distinguishing subjects into high-grade and low-grade disease, as demonstrated with ROC analysis.

(AUC=Area Under curve)

FIG. 12A shows ROC curves of the metrics from flow cytometry and TCRseq data indicated. FIG. 12B shows feature importance scores of indicated variables from a machine learning model using 70 percent of data to train the model (30 percent of the data was then used to test the model). The model was developed with the XGBoost algorithm using multiple metrics derived from flow and TCRseq data. FIG. 12C shows the performance of the trained multivariate classifier when used to classify patients with high vs low grade disease as shown by ROC analysis in test data (30% of all data available from the TCRseq and flow cytometry analysis of the pre-LUSC cohort).

FIG. 13 shows a workflow showing how to combine data obtained from a plurality of T cell analyses

FIG. 14 Top) depicts the pre-invasive data and shows the T cell early detection indices (TEDI) in high grade pre-invasive samples versus low grade samples, Bottom) shows the T cell early detection indices (TEDI) in healthy patients versus NSCLC (LUAD+LUSC) patients (the majority of which have stage I disease) deriving from the ASCENT analysis. Each datapoint is a patient and the p-value is from a one-tailed MW test. This is demonstrated by the TEDI CD39 Ki67 (CD3), where NSCLC patients have a higher proportion of CD3 CD39+Ki67+ cells than healthy.

FIG. 15 shows the T cell early detection indices (TEDI) of the pre-invasive data (i.e., high grade and low-grade samples) and the ASCENT data (i.e., healthy and NSCLC samples) combined. Each datapoint is a patient with the median value shown (top) or the mean of all patients in each group (bottom) and p-values are from a Kruskal-Wallis test, where only significant values are shown. Error bars represent SEM. An increase in the TEDI CD39 Ki67 ratio from healthy samples to low-grade samples to high-grade samples is observed, with the signal peaking at high-grade samples and dropping off at the lung cancer stage.

FIG. 16 shows the lower frequency of naïve CD4+ T cells in NSCLC patients compared with healthy patients (p=0.1, one-tailed MW test). Naïve CD4+ T cells are shown as a percentage of effector CD4+ T cells (eCD4), meaning non-regulatory CD4+ T cells, or FoxP3-CD4+ T cells.

FIG. 17 shows the combined data for (i) healthy and low grade samples (n=66) compared with (ii) high-grade pre-invasive samples and NSCLC patients (N=88). T cell early detection indices (TEDI); % Total Treg, % Treg CD39+, % Tref CD39+Ki67+ and % naïve CD4+ T cells as a proportion of total CD4 cells are all provided. p-values are all from a one-tailed MW test.

FIG. 18 shows the % Treg, Treg CD39+ and Treg CD39+Ki67+ as a proportion of total CD4 cells for both high grade pre-invasive and low grade samples (top) and for healthy and NSCLC patients (bottom). Each datapoint is a sample (top) or patient (bottom) and the p-value is derived from a one-tailed MW. Treg stands for a T regulatory cell, which are CD4+ T cells that express the biomarker FoxP3.

FIG. 19 shows the % Treg, Treg CD39+ and Treg CD39+Ki67+ as a proportion of total CD4 cells for healthy, low grade pre-invasive samples, high grade pre-invasive samples and NSCLC patients. Each datapoint is a patient (healthy/NSCLC) or sample (low-/high-grade) and uncorrected KW p-values are shown for all. Treg stands for a T regulatory cell, which are CD4+ T cells that express the biomarker FoxP3.

FIG. 20 shows a volcano plot of CD8+ T cell populations determined by computational clustering of flow cytometry data of pre-invasive samples that are significantly enriched in high grade (HG; upper right quadrant) pre-invasive or low grade (LG, upper left quadrant) samples.

FIG. 21 shows box plots for naïve and Tem.Prolif.CF39hi populations at the sample level for both patients with both low grade and high grade pre-invasive species. Tem.Prolif.CF39hi cells are significantly enriched in high grade pre-invasive samples, and naïve cells are significantly enriched in low grade pre-invasive samples. Both are shown as a proportion of total CD8+ T cells.

FIG. 22 Top) shows a volcano plot of CD8+ T cell populations determined by computational clustering of flow cytometry data that are significantly enriched in LUSC (upper right quadrant). Bottom) shows a box plot for the Tem.Prolif.CF39hi population which is significantly enriched in LUSC patients compared to a healthy group.

FIG. 23 shows a combined box plot of values from the pre-invasive flow cytometry analysis and the ASCENT flow cytometry analysis to display the change in Tem.Prolif. CD39hi population. Each dot represents a patient (healthy/LUSC) or sample (low/high). Shown as a proportion of total CD8+ T cells.

FIG. 24 shows the flow cytometry gating strategy of CD8+ T cells for the pre-invasive data samples and ASCENT data samples used in Example 9 and FIGS. 20-23. Manual gating frequencies are used to validate results from computational clustering.

FIG. 25 shows the flow cytometry panel used for analysis of the ASCENT data-set.

FIG. 26 shows the D50 diversity index in Top) low and high grade samples, and Bottom) in healthy subjects and patients with NSCLC.

FIG. 27 shows the Top) the Hill number (aka entropy score) in low-grade pre-invasive samples and high grade pre-invasive samples (left), and healthy patients and patients with NSCLC (right), and Bottom) the proportion of repertoire occupied by the top 100 clones in low and high grade pre-invasive samples (left), and healthy subjects and patients with NSCLC (right).

FIG. 28 shows the proportion of repertoire occupied by small (<0.01% of repertoire), medium (0.01-0.1% of repertoire), large (0.1-1% of repertoire) and hyperexpanded (>1% of repertoire) clones in high grade pre-invasive samples, low-grade pre-invasive samples (top), healthy subjects and patients with NSCLC (bottom).

FIG. 29 shows a ratio of large:small clones occupying the repertoire for healthy, low-grade, high grade, and NSCLC patients. Each dot represents the mean value for each group, and error bars represent SEM.

FIG. 30 shows Top) the proportion of repertoire occupied by small (<0.01% of repertoire), medium (0.01-0.1% of repertoire), large (0.1-1% of repertoire) and hyperexpanded (>1% of repertoire) clones, Bottom Left) the large:small clones ratio occupying the repertoire, and Bottom Right) the entropy score (aka Hill number) for healthy subjects and patients with NSCLC generated from a different, publicly-available data-set.

FIG. 31 shows Top Left) the proportion of repertoire occupied by the top 100 clones, Top Right) the D50 Diversity Index and Bottom) the clonality index for healthy subjects and patients with NSCLC generated from a different, publicly-available data-set.

FIG. 32 shows the D50 diversity index, the Hill number/entropy score, the ratio of large:small clones occupying the repertoire and the proportion of repertoire occupied by the top 100 clones for a combined healthy+low grade data, and a combined high grade+NSCLC data. LG=low grade, HG=high grade.

FIG. 33 shows Kaplan-Meier analysis of combined metric from flow cytometry and TCR-seq showing a significant difference when future lung cancer diagnosis is used as outcome.

FIG. 34 Top) shows feature importance scores of indicated variables from a machine learning model developed with the XGBoost algorithm using multiple metrics derived from flow cytometry and TCRseq data. Bottom) shows the ROC analysis of the combination of these techniques alongside standard clinical metrics into a single analysis show promise in distinguishing low-grade from high-grade pre-invasive subjects. The model was tested on ASCENT data (AUC=1) and validated on pre-invasive data (AUC=0.76).

FIG. 35 shows another example flow cytometry manual gating strategy for the manual gating analysis of the pre-invasive CD4+ T cell data (i.e., high grade and low grade pre-invasive samples) and ASCENT flow cytometry CD4+ T cell data (i.e., healthy subjects and NSCLC patients), used in Example 8, including analysis that selects for regulatory T cells using the biomarker FoxP3. All manual gating analysis was carried out on FlowJo v10.8.1. Populations gated are shown on a concatenated file of all samples from one batch. Frequencies from total CD4 were used to validate cluster significance from the high-dimensional clustering pipeline analysis.

FIG. 36 Top) shows the % Treg CD45RA-CD39+ as a proportion of total CD4 cells for both high and low grade pre-invasive samples (left) and for healthy and NSCLC patients (right). Bottom shows a combined data from the pre-invasive flow cytometry analysis and the ASCENT flow cytometry to display the change in Treg CD45RA-CD39+ population.

FIG. 37 shows a Forest plot displaying odds ratios calculated from multivariate analysis logistic regression models accounting for listed clinical variables in the pre-invasive data. This analysis shows that all three metrics from flow cytometry (Treg CD45RA-CD39+) and TCR-seq (large:small ratio, proportion of top 100 clones) remain significant even after accounting for the listed clinical variables.

### DETAILED DESCRIPTION

### Summary of how the Problem is Solved

The present invention provides a blood test which examines the state of blood T cell differentiation to determine whether a subject has or is at risk for having a progressive or high-grade pre-invasive lesion or nodule or progressive small mass. It is understood that progressive or high-grade pre-invasive lesions or nodules or progressive small masses are of concern because they can develop into a solid malignant tumour.

The methods of the present invention can also provide a blood test which examines the state of blood T cell differentiation to determine whether a subject has or is at risk of having a solid malignant tumour. This is because similar or the same patterns of T cell differentiation skewing may be identified if a subject has a solid malignant tumour Therefore the present invention provides a method for determining whether a subject is at risk for having a progressing or high-grade pre-invasive lesion, nodule or small mass, and/or having a solid malignant tumour, by analysing T cell differentiation skewing in a sample of blood obtained from the subject. Prior to the present invention there was no effective non-invasive, screening method for early detection of a progressive or high-grade pre-invasive lesion or nodule or small mass that uses T cell differentiation for the test.

T cells in humans with a challenged immune system are known to look more activated, exhausted or differentiated, compared to those who are not challenged. Prior to the present invention it was not known that such a challenged immune system could be detected in the blood of individuals who do not yet have an established solid malignant tumour but harbour a lesion or nodule or small mass of the type that can be expected to develop into disease. The present invention leverages this concept by generating a test that measures T cell differentiation, exhaustion and activation.

The present invention is concerned with early detection of cancer. Prior art methods have been concerned with detection of cancer at stage 1 or 2, rather than detection at stage 3 or 4 when the prognosis is very poor, and this has sometimes been referred to as “early detection”. In contrast early detection in the context of the present invention includes detection of pre-invasive or high-grade lesions or nodules that can develop into a solid tumour. Detection prior to the establishment of a tumour can lead to greater treatment options and improved prognosis. Detection of such lesions or nodules currently occurs via low dose CT screening or ineffective and/or costly and/or invasive and burdensome methods. A high sensitivity and specificity blood test using T cell differentiation has not been described.

The methods of the present invention may be used for detection of a progressing or high-grade pre-invasive lesion or nodule (or pre-invasive neoplasia). The present invention may also be valuable to detect solid malignant tumours at stage I or later, and in some embodiments, preferably stage I solid malignant tumours.

Pre-invasive generally refers to a cluster of malignant cells or lesions that have not left their original focal or spread to other parts of the body and are not yet considered to be invasive. Nodule generally refers to a growth or lump that may be malignant or benign. The early cancer detection in the present disclosure is concerned with changes prior to stages 1˜4 of established cancer. Therefore, pre-invasive as described herein may refer to a stage of neoplasia development before stages 1˜4 cancer. Optionally, this disclosure may be concerned with very early (stage 1) solid tumours, i.e., the detection of very early (stage 1) solid tumours.

In the context of this invention, pre-invasive lesions or nodules or small masses are or can be classified into low-grade and high-grade. High-grade pre-invasive lesions, nodules or small masses have a well-established meaning in the art. This is demonstrated in at least by Pennycuick et al; Cancer Discov. 2020 October; 10(10): 1489-1499; Banerjee et al; Journal of Thoracic Oncology, Volume 4, Issue 4, April 2009, Pages 545-551; D. Moro-Sibilot et al; European Respiratory Journal 2004 24:24-29, among others.

The term high-grade pre-invasive lesion, nodule or small mass may also encompass or may be used interchangeably with pre-invasive neoplasia and pre-invasive neoplastic lesion. In some embodiments, the term high-grade pre-invasive lesion, nodule or small mass may also encompass or be used interchangeably with the following terms in the Table below:

High grade pre-malignant lesions
High grade preinvasive neoplasia
Severe dysplasia
Carcinoma-in-situ (CIS)
Early lesion, nodule or small mass
Pre-malignant lesion, nodule or small mass
Pre-cursor lesion, nodule or small mass
High-grade dysplasia
Squamous dysplasia
Minimally invasive lesion, nodule or small mass
Stage 0 lesion, nodule, small mass, or tumour
Progressive preinvasive disease
Progressive preinvasive neoplasia
Any of the above with tissue type specific prefixes, suffixes or
combinations which indicate disease involved in carcinogenesis
for different cancer types (e.g. pulmonary or bronchial preinvasive
neoplasia when referring to disease that results in lung cancer).

High-grade lesion is an umbrella term encompassing carcinoma in situ (CIS) and severe dysplasia lesions of the airway. Low-grade lesions include hyperplasia, squamous metaplasia, mild dysplasia, and moderate dysplasia. These are all different histological states of different levels of neoplasia of the airway that can be identified from a lesion biopsy by a histopathologist. These different histological states are classified into low- or high-grade groups to facilitate downstream analysis, and separated in this way based on the clinical risk each group carries. Low-grade lesions carry no significant clinical risk of developing into invasive disease/lung cancer, whereas 50% of high-grade lesions have been reported to progress into invasive disease/lung cancer (P J George et al. Surveillance for the detection of early lung cancer in patients with bronchial dysplasia. Thorax 2007; 62:43-50. doi: 10.1136/thx.2005.052191).

In some embodiments, the high grade pre-invasive lesion, nodule or solid mass may be a high-grade bronchial lesion, a pre-invasive lesion of the bronchus, a bronchial pre-invasive lesion, a bronchial dysplasia, bronchial lesion or an endobronchial lesion.

In the present invention, classification of lesions or nodules or small masses into low-grade or high-grade can be performed using a ratio of differing T cell phenotypes, identified using detection biomarkers on/within the T cells. Classification of lesions or nodules or small masses into low-grade or high-grade can be performed by determining the proportion of T cells comprising one or more detection biomarkers on/within the T cells as a percentage of T cells.

Skewing of T cell differentiation can be detected by analysing a trait of the T cells.

Alternatively, skewing of T cell differentiation can be detected by analysing a plurality of traits of the T cells. The information obtained from analysing a plurality of traits can be combined (e.g., using TCR-seq and cytometry). Combining information from analysing a plurality of traits may provide a further improved method for determining whether a subject is at risk for having a progressing or high-grade pre-invasive lesion, nodule or small mass, or having a solid malignant tumour.

A trait of T cells to be analysed can be a phenotypic trait.

Examples of methods for analysing skewing of T cell differentiation are described herein.

Skewing of T cell differentiation can be detected by determining a ratio of activated and/or exhausted T cells:naïve and/or resting T cells in a sample of blood obtained from the subject, or by determining a proportion of activated and/or exhausted T cells as a percentage of T cells (i.e., total T cells). Or a subpopulation of T cells (i.e., total subpopulation of T cells). Optionally skewing of T cell differentiation can be detected by determining a ratio of activated and/or exhausted T cells:T cells which are not activated and/or exhausted T cells in a sample of blood obtained from the subject. Where a subject is shown to have more activated and/or exhausted T cells (e.g., based on detection of a combination of T cell biomarker expression), this would be indicative of the subject being at risk of having a progressing or high-grade pre-invasive lesion or nodule or small mass, or having a solid malignant tumour (e.g., a stage 1 solid malignant tumour). Optionally skewing of T cell differentiation can be detected by determining a proportion of activated and/or exhausted T cells as a proportion of T cells, or a subpopulation of T cells, in a sample of blood obtained from the subject. Where a subject is shown to have a greater proportion of activated and/or exhausted T cells (e.g., based on detection of a specific biomarker expression), this would be indicative of the subject being at risk of having a progressing or high-grade pre-invasive lesion or nodule or small mass, or having a solid malignant tumour (e.g., a stage 1 solid malignant tumour).

Skewing of T cell differentiation can be detected by analysing the diversity (i.e., diversity and/or clonality) of T cell receptors on the T cells in the sample of blood obtained from the subject. This may include analysing the diversity of CDR3B on TCRs of the T cells in the sample of blood obtained from the subject. Such analysis may be performed using TCR Seq. Where a subject is shown to have a lower or a reduced diversity of the repertoire of TCRs this would be indicative of the subject being at risk of having a progressing or high-grade pre-invasive lesion or nodule or small mass, or having a solid malignant tumour. Since clonality is inversely related to diversity, diversity may be measured directly by one or more diversity metrics or indirectly by one or more clonality metrics. Since clonality is inversely related to diversity, when a subject is shown to have a higher clonality, this would be indicative of the subject being at risk of having a progressing or high-grade pre-invasive lesion or nodule or small mass, or having a solid malignant tumour. Similarly, skewing of T cell differentiation can be detected by analysing the clonality of T cell receptors on the T cells in the sample of blood obtained from the subject, or a proxy of clonality.

Skewing of T cell differentiation can be detected by analysing T cells in a sample of blood obtained from a subject using more than one technique. For example, Therefore, skewing of T cell differentiation can be detected by both:
a) determining a ratio of activated and/or exhausted T cells:naïve and/or resting T cells in a sample of blood obtained from the subject, or by determining a ratio of activated and/or exhausted T cells:T cells which are not activated and/or exhausted T cells in a sample of blood obtained from the subject (for example cytometry), and
b) analysing the diversity and/or clonality of T cell receptors on the T cells in the sample of blood obtained from the subject (TCR Seq).

### Skewing of T Cell Differentiation can Also be Detected by Both:

a) determining a proportion of activated and/or exhausted T cells (or a subpopulation of T cells) as a percentage of T cells (or subpopulation of T cells) in a sample of blood obtained from the subject (for example cytometry), and
b) analysing the diversity and/or clonality of T cell receptors on the T cells in the sample of blood obtained from the subject (TCR Seq).

In the context of the invention, low-grade or “LG”, as used herein, generally refers to the pre-invasive stages of cancer development characterised by squamous metaplasia, mild dysplasia and moderate dysplasia. Generally, a low-grade pre-invasive lesion or nodule or small mass may not need further clinical intervention since it is not expected to develop into cancer.

Whereas high-grade or “HG”, as used herein, generally refers to the pre-invasive disease stages of cancer development characterised by severe dysplasia and carcinoma in situ. Generally, a high-grade pre-invasive lesion or nodule or small mass should be the focus of further clinical follow up. This has a well-established term in the art. This term may encompass or may be used interchangeably with pre-invasive neoplasia and/or other terms as described elsewhere herein.

The utility of the present invention is shown herein in two types of lung cancer with differing clinical and genomic features and in renal cancer. These are cancers with differing body locations and differing mutational backgrounds. Therefore, the underlying principle of T cell activation or T cell differentiation skewing in the presence of a high-grade or progressing pre-invasive nodule, or lesion, which could lead to a malignant tumour is broad and encompasses solid tumours generally. Therefore, the utility of this test for early detection is pan-cancer.

### Advantages

The present invention provides at least the following advantages over previously known methods.
A non-invasive method for detecting whether a subject is at risk for having a progressing or high-grade pre-invasive lesion or nodule or having a solid tumour using T cells as a read out.
A method for distinguishing between a progressing or high-grade pre-invasive lesion or nodule and a non-progressing or low-grade lesion or nodule.
An in vitro method.
A method that only requires a blood sample to determine whether a subject is at risk. Whole blood or peripheral blood mononuclear cell (PBMC) may be used.
A non-invasive procedure that encourages high compliance with a population screening program requiring.
A method that has high sensitivity and specificity.
A method that is valuable for early detection of a wide range of solid tumours, such as RCC, LUAD and LUSC which therefore provides a valuable tool as the test works on a variety of cancers, even when they come from different causes.
A method that can be used as a screening tool on healthy people that can be used to see if the subject is healthy and can be incorporated in standard blood tests to see the likelihood that someone has a risk of progressing or high-grade pre-invasive lesion or nodule or having a solid tumour.
A method that can be personalised to be used as subject's baseline, that can be referred to in later tests to see if the subject's risk of developing progressing or high-grade pre-invasive lesion or nodule or having a solid tumour.
A method that is or can be validated by one or more different techniques (e.g., using flow cytometry and TCR-seq).
A method that can utilise more than one different technique in combination (e.g., using flow cytometry and TCR-seq) to more significantly differentiate between sample types, as a more effective tool for early detection of cancer (e.g., at the pre-invasive stage, or in early stage (e.g., stage 1) cancer.

Unless defined otherwise, all technical and scientific terms used herein have the meaning commonly understood by a person skilled in the art to which this invention belongs. Certain terms are defined below for the sake of clarity and ease of reference.

The present invention provides a method or methods for determining whether a subject is at risk for having a progressing or high-grade pre-invasive lesion or nodule or having a solid tumour. This or these methods can also be understood as distinguishing a subject having a progressing or high-grade pre-invasive lesion or nodule or having a solid tumour, from a subject not having a progressing or high-grade pre-invasive lesion or nodule or not having a solid tumour or having a non-progressing or low-grade pre-invasive lesion or nodule. The method or methods disclosed herein also can distinguish a subject having a progressing or high grade pre-invasive lesion or nodule, or having a solid tumour (e.g., a stage I solid tumour) from a healthy subject.

The subject is a mammal. The subject may be a human. Alternatively, the subject may be in the Primates, Rodentia, Canidae, Felidae, Equidae order or other mammal orders. The subject may be a horse, cat, dog or other companion animal. The subject can be healthy or asymptomatic. The subject may be a patient. The subject can be suspected of having a cancer. The subject can have a genetic pre-disposition to cancer or have lifestyle factors increasing the likelihood for developing cancer. The subject may have previously received treatment for a cancer. The methods of the present invention may also be used for population screening and/or be used as a standard clinical tool in routine blood work, also referred to as mass testing.

A subject is at risk, if the method of the present invention indicates that the subject is likely to have, or has, a high-grade pre-invasive lesion or nodule. A subject is also at risk if the method of the present invention indicates that the subject is likely to have, or has, a solid tumour, e.g., a stage 1 solid malignant tumour.

The term solid tumour generally refers to an abnormal mass of tissue that usually does not contain cysts or liquid areas. Solid tumours may be benign, or malignant. The methods of the present allow to determine if a subject is at risk of developing a solid malignant tumour or at risk of having a solid malignant tumour (e.g., a stage I solid tumour). The term is meant to exclude liquid tumours or haematological malignancies. A lesion generally refers to an area of abnormal tissue. A lesion may be benign or malignant, or premalignant and if premalignant can represent moderate or mild or severe dysplasia, metaplasia or carcinoma in situ. A nodule generally refers to a growth or lump that may be malignant, benign or indeterminate. Low-grade or “LG”, as used herein, generally refers to the pre-invasive disease stages of cancer development characterised by squamous metaplasia, mild dysplasia and moderate dysplasia. Whereas high-grade or “HG”, as used herein, generally refers the pre-invasive disease stages of cancer development characterised by to severe dysplasia and carcinoma in situ. This has a well-established term in the art. This term may encompass or may be used interchangeably with pre-invasive neoplasia and/or other terms as described elsewhere herein.

The methods of the present invention are performed on a blood sample obtained from a subject and hence occur in vitro. The methods can use whole blood. The methods can use PBMCs.

### Analysis of T Cells

The present invention provides a method for determining whether a subject is at risk for having a progressing or high-grade pre-invasive lesion, nodule or small mass, or having a solid malignant tumour (e.g., a stage I solid malignant tumour), the method comprising analysing the proportion of T cells in a sample of blood obtained from the subject which are activated and/or exhausted T cells by analysing a trait of the T cells.

The method may include analysing the proportion of T cells in the sample of blood obtained from the subject which are activated and/or exhausted comprises analysing a plurality of traits of the T cells (e.g., by TCR Seq, and biomarkers expressed by T cells, which may be analysed, for example, by flow cytometry).

A trait of the T cells relates to a property of the T cells which can be analysed. The trait may be a phenotypic trait. The phenotypic trait can be diversity (or diversity or clonality) of T cell receptors, which may be analysed, for example, by TCR Seq. The phenotypic trait can be biomarkers expressed by T cells, which may be analysed, for example, by flow cytometry.

The present invention also embraces analysing a plurality of traits of T cells in a sample of blood obtained from the subject. The phenotypic traits can be diversity (or diversity or clonality) of T cell receptors, which may be analysed, for example, by TCR Seq, and biomarkers expressed by T cells, which may be analysed, for example, by flow cytometry. The diversity or clonality of T cell of T cell receptors (e.g., analysed by TCR Seq) and the biomarkers expressed by T cells (e.g., analysed by flow cytometry) may be determined by any suitable method as described herein.

### Diversity of T Cell Receptors

The present invention provides a method for determining whether a subject is at risk for having a progressing or high-grade pre-invasive lesion, nodule or small mass, or having a solid malignant tumour, the method comprising analysing the proportion of T cells in a sample of blood obtained from the subject which are activated and/or exhausted T cells by analysing a trait of the T cells.

The trait may be a phenotypic trait. The phenotypic trait may be the diversity of T cell receptors on the T cells in the sample of blood, optionally the diversity of CDR3B on TCRs of the T cells in the sample of blood, further optionally using TCR Seq. Since clonality is inversely related to diversity, diversity may include one or more diversity or clonality metrics. Therefore, the phenotypic trait may be the diversity and/or clonality of T cell receptors on the T cells in the sample of blood, optionally the diversity of CDR3B on TCRs of the T cells in the sample of blood, further optionally using TCR Seq.

In some embodiments, the diversity and/or clonality of T cell receptors on the T cells in the sample of blood may be determined using any suitable diversity metric or clonality metric. The term clonality and/or clonality metric may encompass metrics which analyse clonal expansion, therefore, in some embodiments, the clonality of T cell receptors on the T cells in the sample of blood may be determined by analysing clonal expansion of the T cell receptors on the T cells in the sample of blood. Suitable methods of determining the diversity and/or clonality are described in a Table below.

| Term | Definition |
|---|---|
| TCR diversity | Generated by the random and imprecise |
| | rearrangements of the V and J segments |
| | of the TCRa and V, D, and J segments of |
| | the TCRb genes in the thymus. Takes into |
| | account the clonal composition (i.e. the |
| | number of unique TCR sequences) and |
| | the distribution of these sequences (i.e. |
| | their relative abundance). It relates to the |
| | level of uncertainty that a TCR sequence |
| | would be sorted form a repertoire and |
| | would belong to a certain T cell clone, i.e. |
| | a unique TCR sequence |
| TCR clonality | The inverse of diversity. |
| D50 | Diversity metric. Calculates the minimum |
| | number of distinct clonotypes amounting |
| | to greater than or equal to 50% of a total |
| | of sequencing reads obtained following |
| | amplification and sequencing |
| Hill number | A mathematically unified family of |
| | diversity indices, differing among |
| | themselves only by an exponent q; when |
| | q = 1, this is also known as entropy index, |
| | or Shannon entropy, or Shannon index |
| Clonality index | Overall clonality metric of the repertoire. 1 |
| | minus entropy. |
| Proportion of the | Clonality metric measuring the proportion |
| repertoire occupied | of the total repertoire which is occupied by |
| by the top n clones | the top n clones, where n = 50-1000. Here |
| | n = 100 is used. |
| The ratio of the | A clonality metric, derived from a ratio |
| repertoire of TCRs | calculated between large clones |
| occupied by large | (occupying 0.1-1% of the repertoire) and |
| clones:the repertoire | small clones (occupying <0.01% of the |
| of TCRs occupied by | repertoire), relating to clonal expansion. |
| small clones | |

In some embodiments, the subject may be at risk if the diversity of the repertoire of TCRs is less than the diversity of the repertoire of TCRs of a comparison subject or a plurality of comparison subjects. In this context a comparison subject can be a healthy subject, or a plurality of comparison subjects can be plurality of healthy subjects or a member of the general population. In some embodiments and examples described herein, the diversity metric is analysed using a D50 diversity score and/or a Hill number.

In some embodiments, the subject may be at risk if the clonality of the repertoire of TCRs is higher than the clonality of the repertoire of TCRs of a comparison subject or a plurality of comparison subjects. In this context a comparison subject can be a healthy subject, or a plurality of comparison subjects can be plurality of healthy subjects or a member of the general population. In some embodiments and examples described herein, the clonality metric may be analysed using a clonality index, the clonality of the repertoire of the TCRs occupied by the top n clones (e.g., wherein n is 50-1000 clones, or n=100), and/or the ratio of the repertoire of TCRs occupied by large clones:the repertoire of TCRs occupied by small clones.

### The Subject May be at Risk if:

a) the diversity of the repertoire of TCRs is less than the diversity of the repertoire of TCRs of a comparison subject or a plurality of comparison subjects,
b) D50 diversity score is lower than a D50 diversity score obtained from analysing T cells in a sample of blood obtained from a comparison subject or a plurality of comparison subjects,
c) a clonality score is higher than a clonality score obtained from analysing T cells in a sample of blood obtained from a comparison subject or a plurality of comparison subjects, or
d) Hill number is lower than a Hill number obtained from analysing T cells in a sample of blood obtained from a comparison subject or a plurality of comparison subjects, or
e) the clonality of the repertoire of the TCRs occupied by the top n clones is higher than the clonality of the repertoire of TCRs occupied by the top n clones in a comparison subject or a plurality of comparison subjects, wherein n is optionally between 50 and 1000, e.g., wherein n=100
(f) the proportion of the repertoire of TCRs occupied by small clones is lower than the proportion of the repertoire of TCRs occupied by small clones in a comparison subject or a plurality of comparison subjects, or
(g) the proportion of the repertoire of TCRs occupied by large clones is higher than the proportion of the repertoire of TCRs occupied by large clones in a comparison subject or plurality of comparison subjects or
(h) the ratio of the repertoire of TCRs occupied by large clones:the repertoire of TCRs occupied by small clones is higher than the ratio of the repertoire of TCRs occupied by large clones:the repertoire of TCRs occupied by small clones in a comparison subject or a plurality of comparison subjects

In this context a comparison subject can be a healthy subject, or a plurality of comparison subjects can be plurality of healthy subjects or a member of the general population,

### The Subject May be at Risk if:

a) the diversity of the repertoire of TCRs is similar to or greater than the diversity of the repertoire of TCRs of a comparison subject or a plurality of comparison subjects,
b) D50 diversity score is similar to or greater than a D50 diversity score obtained from analysing T cells in a sample of blood obtained from a comparison subject or a plurality of comparison subjects,
c) a clonality score is similar to or greater than a clonality score obtained from analysing T cells in a sample of blood obtained from a comparison subject or a plurality of comparison subjects, or
d) Hill number is similar to or greater than a Hill number obtained from analysing T cells in a sample of blood obtained from a comparison subject or a plurality of comparison subjects.
e) the clonality of the repertoire of TCRs occupied by the top n clones is similar to or lower than the clonality of the repertoire of TCRs occupied by the top n clones in a comparison subject or a plurality of comparison subjects, optionally wherein n is 50 to 1000, e.g., wherein n=100
(f) the proportion of the repertoire of TCRs occupied by small clones is similar to or greater than the proportion of the repertoire of TCRs occupied by small clones in a comparison subject or a plurality of comparison subjects, or
(g) the proportion of the repertoire of TCRs occupied by large clones is similar to or lower than the proportion of the repertoire of TCRs occupied by large clones in a comparison subject or plurality of comparison subjects or
(h) the ratio of the repertoire of TCRs occupied by large clones:the repertoire of TCRs occupied by small clones is similar to or lower than the ratio of the repertoire of TCRs occupied by large clones:the repertoire of TCRs occupied by small clones in a comparison subject or a plurality of comparison subjects.

In this context a comparison subject or a plurality of comparison subjects are those known to can be a subject known to have a progressing or high-grade pre-invasive lesion, nodule or small mass, or known to have a solid malignant tumour.

Based on the above, the person skilled in the art of TCR Seq knows whether identifying greater, similar or lower diversity of TCRs is indicative of the subject being at risk based on the disease status (having cancer or being healthy) of the comparison subject(s).

The term “T cell receptor” (TCR), as used herein, generally refers to the molecular structure on T cells that is composed of two different protein chains, with the vast majority of human T cells having TCR containing α (alpha) and β (beta) chains and minority comes from TCR containing γ (gamma) chain and one δ (delta) chains. In the majority of T cells, diversity of TCR comes from genes encoding alpha (Tcra) and beta chains (Tcrb) having multiple non-contiguous gene segments which include variable (V) and joining (J) for the Tcra gene and variable (V), diversity (D), and joining (J) segments for the Tcrb gene. The process of genetic recombination of the DNA encoded segments in individual T cells occurs, that results in random combinations of the V, D, and J segments for TCR beta chains and V and J segments for TCR alpha chains. This results in formation of CDR3, which is essential for the T cell's ability to recognise its cognate antigen and is therefore highly variable.

The term “TCRseq”, as used herein, generally refers to the well-established, high throughput sequencing method for studying TCR repertoires, including allowing greater insight into CDR3 diversity within a T cell population. Here DNA or RNA starting material is combined with PCR-based amplification methods. One such method is multiplex PCR, where primers for the J alleles and all known V alleles are combined with the sample to amplify the V and J alleles. These amplified products are then combined with appropriate sequencing adaptors and analysed via next generation sequencing (NGS). The data obtained from the NGS provides information into variables such as the abundance of a particular T cell clone.

The term “repertoire” as used herein, generally refers to the unique T-cell receptor (TCR) genetic rearrangements within the adaptive immune system. In the context of the invention, this includes determining the abundance of a given TCR (and therefore determining the diversity of TCR) within a subject's blood sample, which when compared to a comparison subject, it may indicate the subject is at risk for having a progressing or high-grade pre-invasive lesion, nodule or small mass, or having a solid malignant tumour.

The terms “D50”, “Hill number” and “clonality score or “clonality index”, “proportion of the repertoire occupied by the top n clones, e.g., top 100 clones” or grouping of TCR clonotypes as small, medium, large or hyperexpanded, as used herein, will be understood by the person skilled in the art to be values that are relevant to TCRseq analysis when determining the diversity and/or clonality of the repertoire of TCR in a sample. They may be determined according to the methods described herein (i.e., in the Examples section). For example, the Hill score and the D50 score may be determined using the “repDiversity” function from the immunarch package (v0.6.9). To determine the grouping of TCR clonotypes as small clones (defined as <0.01% of repertoire), medium clones (defined as 0.01-0.1% of repertoire), large clones (defined as 0.1-1% of repertoire) or hyperexpanded clones (defined as >1% of repertoire), the “repClonality” function may be used from the immunarch package (v0.6.9), e.g., as described herein.

To calculate the large:small ratio, the proportion of clones deemed as large (0.1-1% of t repertoire) can be divided by the proportion of clones deemed as small (<0.001% of the repertoire), e.g., as described herein. The proportion of the repertoire occupied by the top n clones may be determined by taking the top n clones from each patient and clustered using the “gliph2” function from the turbogliph package (v0.99.2), e.g., as described herein, wherein n may be from 50 to 1000, and in an example, n is 100. The clonality index may be determined using the entropy package (v1.3.1), e.g., as described herein, using the (1-[‘entropy’ function]). The Hill number described herein may also be known as, and be referred to as, the entropy score, Shannon index or Shannon entropy score herein.

The person skilled in the art will know that based on the comparison of a given subject, a subject's D50, Hill number and/or clonality scores (including the clonality index, The proportion of the repertoire occupied by the top n clones, and the ratio of large:small clonotypes in the repertoire) may be higher or lower than a comparison subject, depending on whether they are a healthy subject or a plurality of healthy subjects, whether the subject is assessed at a different time point, and/or relative to a plurality of subjects from the general population. A skilled person would also be aware that measures of TCR diversity or clonality can also be assessed through alternative metrics or techniques and that metrics that measure with TCR sequence similarity in a sample could provide similar results.

### Cytometry Techniques

Provided in the present disclosure is an example of generating a ratio of T cell differentiation that can be used to classify a subject as having non-progressing or low-grade pre-invasive lung lesions (posing no risk of cancer) or a subject having a progressing or high-grade pre-invasive lung lesions (high risk of developing cancer), a healthy subject or cancer (e.g., stage I cancer). Provided in the present disclosure is also an example of determining the proportion of T cells (or subpopulation of T cells) that are activated and/or exhausted as a percentage of T cells (or a subpopulation of T cells) that can be used to classify a subject has having a non-progressing or low-grade pre-invasive lung lesions (posing no risk of cancer) or a subject having a progressing or high-grade pre-invasive lung lesions (high risk of developing cancer), a healthy subject or cancer (e.g., stage I cancer)

In one embodiment, I method of the present invention comprises determining a ratio of activated and/or exhausted T cells:naïve and/or resting T cells in a sample of blood obtained from the subject, wherein the determining comprises analysing T cells using cytometry to detect the presence or absence of a panel of biomarkers comprising Ki67 and CD39.

Optionally the method of the present invention comprises determining a ratio of activated and/or exhausted T cells:T cells which are not activated and/or exhausted T cells in a sample of blood obtained from the subject, wherein the determining comprises analysing T cells using cytometry to detect the presence or absence of a panel of biomarkers comprising Ki67 and CD39.

A subject having more T cells showing Ki67 and CD39 expression, vs T cells in which there is no detection of expression of one or both of Ki67 and CD39, in comparison with a comparison subject or a plurality of comparison subjects is considered to be at risk of having a progressing or high-grade pre-invasive lesion or nodule. It is also noted that a subject having more T cells showing Ki67 and CD39 expression, vs T cells in which there is no detection of expression of one or both of Ki67 and CD39, in comparison with a comparison subject or subjects is considered to be at risk of having a solid tumour (e.g., a stage 1 solid tumour). In this context the comparison subject or subjects can include:
a healthy subject or a plurality of healthy subjects,
the subject at a different time point,
a plurality of subjects from the general population.

A subject having the same amount or more T cells showing Ki67 and CD39 expression, vs T cells in which there is no detection of expression of one or both of Ki67 and CD39, in comparison with a comparison subject or a plurality of comparison subjects is considered to be at risk of having a progressing or high-grade pre-invasive lesion or nodule. It is also noted that a subject having the same amount or more T cells showing Ki67 and CD39 expression, vs T cells in which there is no detection of expression of one or both of Ki67 and CD39, in comparison with a comparison subject or subjects is considered to be at risk of having a solid tumour (e.g., a stage 1 solid tumour). In this context, the comparison subject or subjects can include:
a subject or a plurality of subjects known or have a solid tumour or known to have a progression or high-grade pre-invasive lesion or nodule,
the subject at a different time point,
a plurality of subjects from the general population.

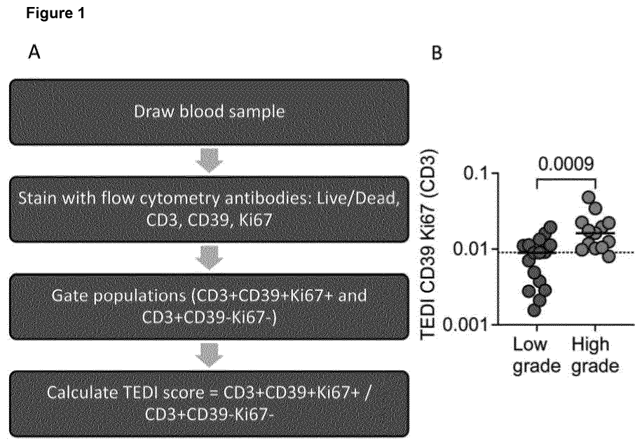

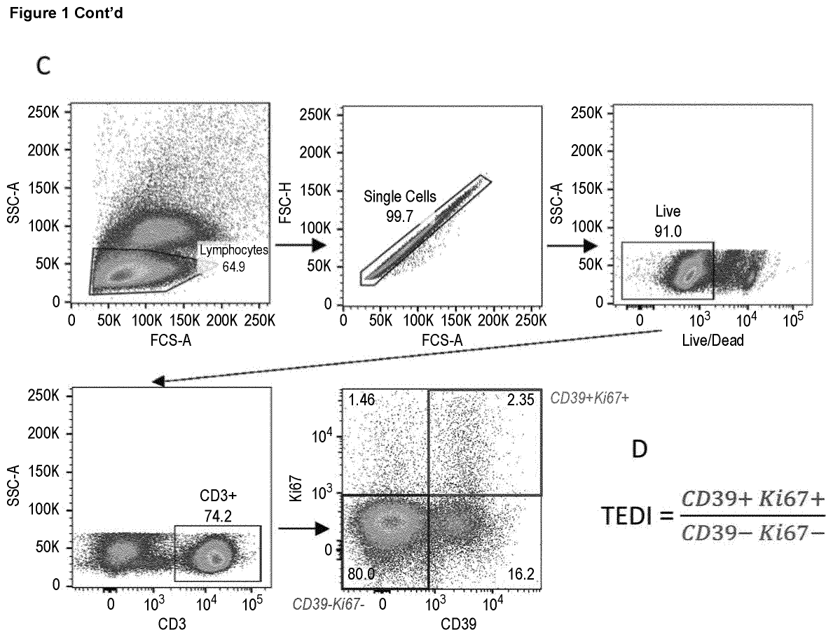

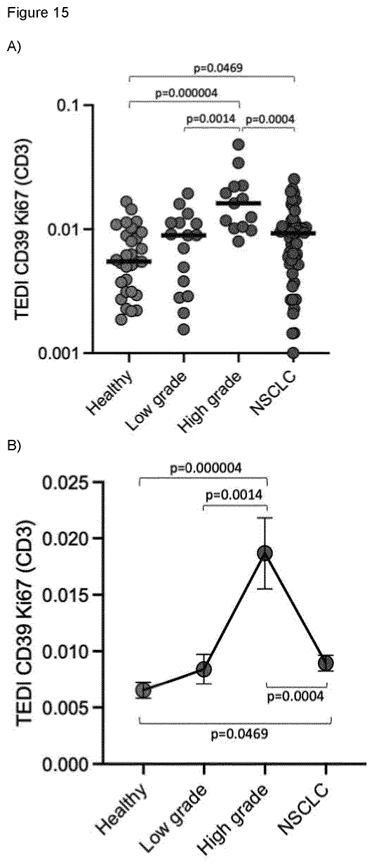

The validity of comparing results in this way is supported through the data provided herein where subjects with known progressing or high-grade pre-invasive lesion or nodule or having a solid tumour have more T cells showing Ki67 and CD39 expression, and conversely, subjects with low-grade disease have less T cells with Ki67 and CD39 expression (FIG. 1), as well as healthy subjects (FIG. 15).

In another embodiment, the method of the present invention comprises determining the proportion of activated and/or exhausted T cells as a percentage of T cells in the blood obtained from the subject, wherein the determining comprises analysing T cells using cytometry to detect the presence or absence of a panel of biomarkers comprising FoxP3, wherein the T cells are CD4 T cells. In some embodiments, the panel of biomarkers comprises CD4 to identify the CD4 T cells also using cytometry. In some embodiments, the CD4 T cells are pre-selected for before the cytometry step, e.g., using a T cell enrichment or purification kit and magnetic selection (as described elsewhere herein). In some embodiments, the panel of biomarkers further comprises CD39. In some embodiments, the panel of biomarkers further comprises CD39 and Ki67, or CD39 and CD45RA. In some embodiments, the activated and/or exhausted T cells express FoxP3 and CD39, wherein the T cells are CD4 T cells. In some embodiments, the activated and/or exhausted T cells express FoxP3 and CD39 in combination with Ki67, wherein the T cells are CD4 T cells. In some embodiments, the activated and/or exhausted T cells express FoxP3 and CD39 and do not express CD45RA, wherein the T cells are CD4 T cells.

In another embodiment, the method of the present invention comprises determining the proportion of CD4 regulatory T cells as a percentage of CD4 T cells in the blood obtained from the subject, wherein the determining comprises analysing CD4 T cells using cytometry to detect the presence or absence of a panel of biomarkers comprising FoxP3. In some embodiments, the panel of biomarkers further comprises CD39. In some embodiments, the panel of biomarkers further comprises CD39 and Ki67, or CD39 and CD45RA. In some embodiments, the CD4 regulatory T cells are proliferating CD4 regulatory cells and further express CD39 and Ki67.

In another embodiment, the method of the present invention comprises determining the proportion of regulatory T cells as a percentage of T cells in the blood obtained from the subject, wherein the determining comprises analysing T cells using cytometry to detect the presence or absence of a panel of biomarkers comprising FoxP3, wherein the T cells are CD4 T cells. In some embodiments, the activated and/or exhausted T cells express FoxP3 and CD39, wherein the T cells are CD4 T cells. In some embodiments, the activated and/or exhausted T cells express FoxP3 and CD39 in combination with Ki67 (and optionally CD4), wherein the T cells are CD4 T cells. In some embodiments, the activated and/or exhausted T cells express FoxP3 and CD39 and do not express CD45RA, wherein the T cells are CD4 T cells.

A subject having more CD4+ T cells showing FoxP3 expression (preferably in combination with CD39 expression and further preferably in combination with (i) Ki67 expression, or (ii) no detection and/or no expression of CD45RA), vs CD4 T cells in which there is no detection of expression of FoxP3 (preferably in combination with no detection or expression of CD39, and further preferably in combination with (i) no detection or expression of Ki67, or (ii) expression and/or detection of CD45RA, in comparison with a comparison subject or a plurality of comparison subjects is considered to be at risk of having a progressing or high-grade pre-invasive lesion or nodule. It is also noted that a subject having more CD4+ T cells showing FoxP3 expression (preferably in combination with CD39 expression and further preferably in combination with (i) Ki67 expression, or (ii) no detection and/or expression of CD45RA), vs CD4+ T cells in which there is no detection of expression of FoxP3 (preferably in combination with no detection or expression of CD39, and further preferably in combination with (i) no detection or expression of Ki67, or (ii) expression of CD45RA), in comparison with a comparison subject or subjects is considered to be at risk of having a solid tumour (e.g., a stage 1 solid tumour). In this context the comparison subject or subjects can include:
a healthy subject or a plurality of healthy subjects,
the subject at a different time point,
a plurality of subjects from the general population.

A subject having the same amount or more T cells showing Ki67 and CD39 expression, vs T cells in which there is no detection of expression of one or both of Ki67 and CD39, in comparison with a comparison subject or a plurality of comparison subjects is considered to be at risk of having a progressing or high-grade pre-invasive lesion or nodule. It is also noted that a subject having the same amount or more T cells showing Ki67 and CD39 expression, vs T cells in which there is no detection of expression of one or both of Ki67 and CD39, in comparison with a comparison subject or subjects is considered to be at risk of having a solid tumour. In this context, the comparison subject or subjects can include:
a subject or a plurality of subjects known or have a solid tumour or known to have a progression or high-grade pre-invasive lesion or nodule,
the subject at a different time point,
a plurality of subjects from the general population.

The methods of the present invention are valuable for early detection of a variety of solid tumours, including stage 1 solid tumours/stage 1 solid malignant tumours. Early detection may be for NSCLC including LUAD or LUSC, mesothelioma, renal cancer, melanoma, pancreatic cancer, head and neck cancer, prostate cancer, brain cancer including glioblastoma, breast cancer, bowel cancer, liver cancer.

A ratio as used herein, generally refers to the relationship of the frequency of activated and/or exhausted T cells compared with the frequency of naïve and/or resting T cells in a subject. Alternatively, a ratio can refer to the relationship of the frequency of activated and/or exhausted T cells amongst all T cells or amongst a subset of T cells in a subject (i.e., the proportion of activated/exhausted cells as a percentage of T cells, or amongst a subset of T cells in a subject, e.g., in a sample of blood in a subject).

A ratio indicative of a whether a subject is at risk can be inferred from having a high percentage of activated/exhausted T cells in a sample obtained from the subject, or a percentage of activated/exhausted T cells in a sample obtained from the subject which is greater than the average percentage of activated and/or exhausted T cells in a comparison subject or plurality of subjects is indicative of a subject at risk.

An activated T cell as used herein, generally refers to a T cell that has recognised its cognate antigen, on a target cell expressing the antigen including an epithelial cell or other cell that may progress to cancer or be part of a lesion or nodule or tumour triggering activation and/or proliferation of that T cell. Repetitive encounter of an antigen or a high affinity interaction with that antigen can cause terminal differentiation. Chronic antigen stimulation with or without inhibitory signalling or defective co-stimulation with or without inflammation may result in T cell exhaustion or T cell dysfunction. An activated T cell may or may not be proliferating.

### Activated T Cells May Include:

Any subset of CD4 or CD8 T cell or CD3 positive cell expressing CD39 and/or Ki67 in the presence or absence of other commonly used activation markers obvious to a skilled researcher.
CD8 Terminally differentiated effector memory cells (RA) that are activated (TEMRA.Act). Such cells may have one or more of the biomarkers CD39, Ki67 CD45RA or CD57.
CD4 Regulatory T cells that are proliferating and may express PD-1 (Teg.prolif.PD-1) and may express one or more of the biomarkers CD39, Ki67 or PD-1 (i.e., along with FoxP3).
CD4 Effector memory CD4 T cells (TEM) which may express one or more of the biomarkers CD39 and Ki67. Such cells may lack the biomarkers CCR7 and CD45RA.
CD4 Regulatory T cells (Treg) which may express one or more of the biomarkers CD39 and Ki67 (i.e., along with FoxP3).
CD4 Proliferating regulatory T cells (Treg.prolif) may express one or more of the biomarkers CD39, Ki67hi or PD-1 (i.e., along with FoxP3).
CD4 Proliferating effector memory CD4 T cells (Tem.Prolif) which may express one or more of the biomarkers CD39, Ki67hi and such cells may lack one or more of the biomarkers CCR7 and CD45RA.
cytolytic CD4 T cells (Cytolytic) which may be terminally differentiated and cytotoxic CD4 T cells and may have the biomarker CD57.

A proliferating T cell, as used herein, generally refers to a T cell that has interacted with their cognate antigen or other activating stimuli (thereby becoming activated) and has increased, are increasing or in the process of increasing the numbers of that T cell through cell growth and division. A proliferating T cell is usually activated but may not always be.

An exhausted T cell as used herein, generally refers to a state of T cell dysfunction which can be defined by biological changes in the T cell, including poor effector function and/or expression of inhibitory receptors and a transcriptional state and/or phenotype and/or epigenetic state which is distinct from that of effector or memory or naïve T cells. Exhausted T cells may be referred to as Tex cells, dysfunctional T cells or Tdys cells.

### Exhausted T Cells May Include:

exhausted, proliferating CD8 T cells (Tex. Prolif) which may express one or more of the biomarkers PD-1, Ki67 or CD39.
Exhausted, proliferating CD4 T cells (Tex. Prolif) which may express one or more of the biomarkers PD-1, Ki67hi or CD39.
Progenitor, exhausted CD4 T cells (TPEX), an early version of exhausted CD4 T cells and may express one or more of the biomarkers CD39, Ki67 or PD-1.

A naïve T cell, as used herein, generally refers to a T cell, which may be a CD4+ helper T cell or a CD8+ cytotoxic T cell, that has not yet encountered their cognate antigen.

### Naïve T Cells May Include:

naïve CD8 T cells (Naïve) which may express one or more of the biomarkers CD45RA, CCR7.
Naïve CD4 T cells that may have lost TCF7 expression (Naïve. TCF7neg), and/or which may express one or more of the biomarkers CD45RA, CCR7 or CD27.
CD4 native T cells (Naïve) which may express one or more of the biomarkers CD45RA and CCR7.

A resting T cell, as used herein, generally refers to a quiescent T cell that has stopped or is not proliferating or expressing biomarkers of activation.

### Resting T Cells Includes:

CD8 terminally differentiated effector memory cells (RA) (TEMRA.Rest) which may express the biomarker CD45RA. Such cells may lack one or more of the biomarkers CCR7 and CD57.
Resting central memory CD4 T cells which are an early differentiated resting memory cells that are unstimulated, which may express the biomarker CCR7. Such cells may lack one or more of CD45RA and CD38.

In some methods, the analysis may be performed on a T cell subtype or subpopulation. In some embodiments, the analysis may be performed on CD3 T cells, CD4 T cells, CD8 T cells, CD3+CD8+ T cells, or CD4+CD3+ T cells.

In some embodiments, the T cell subtype or subpopulation may be regulatory T cells. Regulatory T cells express the FoxP3 biomarker. In some embodiments, prior to analysis, a preliminary step of identifying regulatory T cells may be performed, e.g., using an antibody specific to the biomarkers FoxP3. This analysis is performed on CD4 T cells. In some embodiments, prior to analysis, a preliminary step of identify CD4 T cells may be performed, e.g., using an antibody specific to CD4.

In some embodiments, the T cell subtype or subpopulation may be an effector memory cell (Teffm). effector memory T cells may be absent of CCD7, CD45RA and/or PD1 biomarkers. In some embodiments, prior to analysis, a preliminary step of identifying an effector memory cell may be performed, e.g., using one or more antibodies specific to CCD7, CD45RA, and/or PD1. This analysis may be performed in CD8 T cells, or CD3+CD8+ T cells.

The methods of the present invention can detect biomarkers using cytometry, which as used herein, generally refers to techniques for measurement of properties of the cells. Cytometry embraces flow cytometry, spectral cytometry and mass cytometry (including time of flight cytometry or cytometry by time of flight (CyTOF).

The cytometry can be flow cytometry. Flow cytometry refers to a well-established technique that can be used to detect and analyse populations of cells and provide information regarding the physical or chemical characteristics of those cells. This detection and/or analysis includes identifying subsets of cells within a population of cells to be analysed that express a specific biomarker or specific biomarker combinations. A biomarker may be a cell surface antigen or may be an intracellular molecule. To identify subsets of cells, one application of flow cytometry may involve performing multi-coloured analysis using antibodies each of which targets biomarker of interest (e.g., according to the examples disclosed herein). Single cells are transferred in the flow cytometer through a stream of fluid and passed by a light source which may activate a fluorescent antibody bound to a cell flowing past the light source. Fluorescent light emitted by an antibody may be detected by an electronic detection apparatus within the flow cytometer that in turn relays this information computationally through creation of gates/gating that classifies cells that are expressing a biomarker and those that are not. In some examples, gating is performed using FlowJo software, for example, FlowJo v10.8.1 software, although other gating software may be used. Typically, a negative control, such as an antibody for a biomarker that is not expressed on the population of cells being analysed is used to set the gating for cells that will be classified as not expressing a biomarker. The set gating is then used for the rest of the antibody panel for the biomarkers of interest. The information is then visualised to correlate the amount of detected fluorescence with the number of cells that are expressing the biomarker of interest and the amount of the biomarker being expressed by those cells. This technique provides a fast, sensitive and high-throughput analysis of a population of cells. Flow cytometry may be used to profile the frequency and intensity of biomarkers expressed by T cells.

The biomarkers to be detected are biomarkers associated with T cell activation, exhaustion, regulatory or memory differentiation. Detection of a biomarker or a specific combination of biomarkers can be used to determine the strength, frequency and type of immune activation. Therefore, in embodiments where flow cytometry, or spectral or time of flight or another form of cytometry is used to detect the presence or absence of a presence of biomarkers, the detecting comprises detecting the signal of one or more fluorescently labelled antibodies that bind to the panel of biomarkers of interest. For example, when the panel or biomarkers includes (i) Ki67—the detecting may comprise detecting the signal of a fluorescently labelled Anti-Ki67 antibody, and/or (ii) CD39, the detecting may comprise detecting the signal of a fluorescently labelled Anti-CD39 antibody, and/or (iii) for FoxP3, the detecting may comprise detecting the signal of a fluorescently labelled Anti-FoxP3 antibody. The presence and/or absence of the other named biomarkers herein, when used in the panel of biomarkers, can also be determined by similarly detecting the signal of a fluorescent labelled antibody targeted against the specific biomarker of interest.

Cytometry also includes spectral cytometry or spectral flow cytometry a technique in which an emission spectrum of multiple fluorescing molecules may be captured by a set of detectors across a defined wavelength range. The fluorescence spectrum can be recognized, recorded as a spectral signature, and used as a reference in multicolour applications.

Cytometry also includes mass cytometry which is a variation of flow cytometry in which antibodies are labelled with heavy metal isotopes rather than fluorochromes. The isotopes are analysed through the cells being ionised and the ions are separated by their mass-to-charge ratio and detected as an electrical signal at the terminal gate of the spectrometer. The readout is typically by time-of-flight mass spectrometry.

In the methods of the present invention the analysis may comprise using at least one of flow cytometry, spectral cytometry and/or mass cytometry, optionally flow cytometry.

In some embodiments, the cytometry analysis (e.g., the flow cytometry analysis) comprises using computational clustering for a particular subpopulation of T cells and/or T cells which express or which do not express one or more particular biomarkers of interest. Example computational clustering strategies are described in further detail in the Examples section herein. The computational clustering strategies described herein are merely an example and the person skilled in the art would be aware of the various software packages and protocols that could be used to analyse the cytometry data and obtain the same biological result.

In some embodiments, the cytometry analysis (e.g., flow cytometry analysis) comprises gating or gating for a particular subpopulation of T cells and/or T cells which express or which do not express a particular biomarker of interest. The terms “gating” or “gates”, as used herein, generally refers to the use of software that is associated with flow cytometry, such as manually drawing two-dimensional gates with a mouse on a computer screen, based on the density contour lines that are provided by software tools. Gates are typically used to distinguish between cells that are expressing a biomarker of interest and those that are not. The cells falling in a gate may be selected (gated in) or excluded (gated out) and the process may be repeated for different two-dimensional (or higher) projections of the gated cells (e.g., two-dimensional dot plots), thus resulting in a sequence of gates that describe subpopulations of the multivariate flow cytometry data. Specific gating strategies are described in further detail in the Examples section herein. The specific gating strategies described herein are merely an example and the person skilled in the art would be aware of the various software packages and protocols that could be used to analyse the flow cytometry data and obtain the same biological result. In some examples, gating (i.e., manual gating) is performed using FlowJo software, for example, FlowJo v10.8.1 software, although other gating software may be used.

A biomarker as used herein, is as a phenotypic biomarker that reflects the activation or differentiation state of a T cell in human blood. Biomarkers that may be detected in the methods of the present invention can include Ki67 and CD39 and/or FoxP3. The methods detect the presence or absence of a panel of biomarkers comprising Ki67 and CD39, and/or FoxP3, optionally in combination with other biomarkers listed herein. Hence the panel of biomarkers of the present invention comprises Ki67 and CD39 and/or FoxP3, optionally in combination with other biomarkers listed herein.

An activated and/or exhausted T cell may express CD39 and Ki67 (CD39+ and Ki67+ T cells). Detection of expression of CD39 and Ki67 is considered to indicate a T cell that is activated and/or exhausted. An activated and/or exhausted T cell may also be a CD4 Regulatory T cell. A CD4 Regulatory T cell expresses the biomarker FoxP3. In some embodiments, the CD4 Regulatory T cell also expresses CD39 (i.e., in combination with FoxP3). Detection of FoxP3 expression (and preferably in combination with CD39) can indicate a CD4+ T cell that is activated and/or exhausted. A CD4+ T cell that expresses FoxP3 is otherwise referred to as a T regulatory cell herein. In some embodiments, the CD4 Regulatory T cell may express FoxP3, Ki67 and CD39. In some embodiments, the CD4 Regulatory T cell may express FoxP3, CD39, and may lack CD45RA.

A naïve and/or resting T cell does not express CD39 or Ki67 (CD39− and Ki67− cells). Lack of detection of expression is CD39 and Ki67 is or can be considered to indicate a T cell is naïve and/or resting.

T cells which are not activated and/or exhausted do not express one or both of CD39 or Ki67 (CD39− and Ki67+ T cells, CD39+ and Ki67− T cells, or CD39− and Ki67− T cells). Lack of detection of expression of one or both of CD39 or Ki67 is or can be considered to indicate a T cell that is not an activated and/or exhausted T cell.

In this disclosure reference to a T cell not expressing a biomarker marker can refer a lack of detection of expression of that biomarker.

The panel of biomarkers may comprise further biomarkers (i.e., in addition to Ki67, CD39 and/or FoxP3). The panel of biomarkers may include one or more further biomarkers selected from CD45RA, CCR7, PD-1, CD57 or CD38. These biomarkers are considered to further assist in distinguishing between activated and/or exhausted T cells and naïve and/or resting T cells.

A panel of biomarkers may comprise Ki67, CD39 and CD45RA. In some embodiments, the activated and/or exhausted cells express Ki67 and CD39 but may or may not express CD45RA.

A panel of biomarkers may comprise Ki67, CD39 and CCR7. In some embodiments, the activated and/or exhausted cells express Ki67 and CD39 but may or may not express CCR7.

A panel of biomarkers may comprise Ki67, CD39 and PD-1. In some embodiments, the activated and/or exhausted cells express Ki67 and CD39 but may or may not express PD-1

A panel of biomarkers may comprise Ki67, CD39 and CD57. In some embodiments, the activated and/or exhausted cells express Ki67 and CD39 but may or may not express CD57.

A panel of biomarkers may comprise Ki67, CD39 and CD38. In some embodiments, the activated and/or exhausted cells express Ki67 and CD39 but may or may not express CD38.

A panel of biomarkers may comprise Ki67, CD39, CD45RA and CCR7. In some embodiments, the activated and/or exhausted cells express Ki67 and CD39 but may or may not express CD45RA and may or may not express CCR7.

A panel of biomarkers may comprise Ki67, CD39, CD45RA, CCR7, and PD-1. In some embodiments, the activated and/or exhausted cells express Ki67 and CD39 but may or may not express CD45RA, may or may not express CCR7, and may or may not express PD-1.

A panel of biomarkers may comprise Ki67, CD39, CD45RA, CCR7, PD-1 and CD57. In some embodiments, the activated and/or exhausted cells express Ki67 and CD39 but may or may not express CD45RA, may or may not express CCR7, may or may not express PD-1 and may or may not express CD57.

A panel of biomarkers may comprise Ki67, CD39, CD45RA, CCR7, PD-1, CD57 and CD38. In some embodiments, the activated and/or exhausted cells express Ki67 and CD39 but may or may not express CD45RA, may or may not express CCR7, may or may not express PD-1, may or may not express CD57 and may or may not express CD38.

A panel of biomarkers may comprise Ki67, CD39, CD45RA, CCR7, CD57, and CD38. In some embodiments, the activated and/or exhausted cells express Ki67 and CD39 but may or may not express CD45RA, may or may not express CCR7, may or may not express CD57 and may or may not express CD38.

A panel of biomarkers may comprise Ki67, CD39, CD45RA, PD-1 and CD57. In some embodiments, the activated and/or exhausted cells express Ki67 and CD39 but may or may not express CD45RA, may or may not express PD-1 and may or may not express CD-57.

In some embodiments, the activated and/or exhausted cells express Ki67 and CD39 but do not express CD45RA. In some embodiments, the activated and/or exhausted cells express Ki67 and CD39 but do not express PD-1. In some embodiments, the activated and/or exhausted cells express Ki67 and CD39 but do not express CCR7. In some embodiments, the activated and/or exhausted cells express Ki67 and CD39 but do not express CD45RA, CCR7 and PD-1. The activated and/or exhausted T cells may CD8+ T cells, or CD8+CD3+ T cells.

The above panel of biomarkers may be combined with (i) CD3, CD4 or CD8, or (ii) CD3 and CD4 or (iii) CD3 and CD8. The presence of CD3 identifies live T-cells. The presence of CD4 identifies CD4+ T cells, i.e., T helper cells. The presence of CD8 identifies CD8+ T cells, i.e., cytotoxic T cells.

A panel of biomarkers may comprise FoxP3 (and optionally CD4). In some embodiments, the activated and/or exhausted cells are CD4+ T cells that express FoxP3.

A panel of biomarkers may comprise FoxP3, and CD39 (and optionally CD4). In some embodiments, the activated and/or exhausted cells are CD4+ T cells that express FoxP3 and CD39.

A panel of biomarkers may comprise FoxP3, CD39 and Ki67 and optionally CD4). In some embodiments, the activated and/or exhausted cells are CD4+ T cells that express FoxP3, CD39 and Ki67.

A panel of biomarkers may comprise FoxP3, CD39 and CD45RA (and optionally CD4). In some embodiments, the activated and/or exhausted cells are CD4+ T cells that express FoxP3, CD39 and do not express CD45RA.

The above panel of biomarkers may be combined with CD3.

Ki67 generally refers to the proliferation biomarker protein Ki-67, encoded by the MKI67 gene and is widely used as a biomarker to assess cell proliferation.

CD39 generally refers to Cluster of Differentiation 39, encoded by the ENTPD1 gene and is a ectonucleoside triphosphate diphosphohydrolase.

CD45RA generally refers to the long isoform of the cell-surface tyrosine phosphatase CD45 (lymphocyte common antigen) encoded by the PTPRC gene.

CCR7 generally refers to C—C motif chemokine receptor 7 encoded by the CCR7 gene. It is a member of the G protein-coupled receptor family.

PD-1 (also known as PD1) generally refers to Programmed cell death protein1, encoded by the PDCD1 gene and is a inhibitory receptor on antigen activated T-cells that plays a critical role in induction and maintenance of immune tolerance to self.

CD38, generally refers to Cluster of Differentiation 38 encoded by the CD38 gene and is a cyclic ADP ribose hydrolase found on the surface of many immune cells, including lymphocytes.

FoxP3 is a member of the forkhead transcription factor family and is a master regulatory of the regulatory pathway in the development and function of regulatory T cells.

The term “hi” as used herein, generally refers to a higher/greater abundance of a particular biomarker relative to other T cell types. The term “lo” as used herein, generally refers to a lower/less abundance of a particular biomarker relative to other T cell types. The term “prolif” as used herein generally refers to a proliferating T cell.

Biomarkers may be measured by directly conjugated antibodies which are available from multiple commercial sources. Alternatively, the skilled person may use a primary antibody specific to the biomarker and a secondary antibody to detect the biomarker indirectly. Antibodies may be used to stain peripheral blood mononuclear cells (PBMCs) isolated from whole blood by centrifugation. Staining may also be performed on whole blood. The biomarker profiles (the frequency of cells expressing the biomarker) can be determined by cytometry using instrumentation and software known to the person skilled in the art. Specific antibodies to detect biomarkers of T cell activation or proliferation state may be used. These antibodies may be:
1. Anti-CD39 (to identify activated and exhausted T cells [Tex], tumour reactive T cells)
2. Anti-Ki67 (to identify proliferating T cells) and/or
3. Anti-FoxP3 (to identify the biomarker FoxP3, which can be used to identify CD4+ T regulatory cells, e.g., with the antibody Anti-CD4)

As described above additional biomarkers may be detected and methods may use one or more the following antibodies:
4. Anti-CD4, to identify the major CD4+ T cell lineage
5. Anti-CD8, to identify the major CD8+ T cell lineage
6. Anti-CD57 (to identify terminally differentiated T cells)
7. Anti-CD38 (to identify activated and exhausted T cells)
8. Anti-CD45RA (to identify Naïve and terminally differentiated T cells)
9. Anti-PD-1 (to identify activated and exhausted T cells)
10. Anti-CCR7 (to identify naïve and early differentiated memory cells).

In some methods, a preliminary step of identifying live T cells for analysis may be performed. This step may involve antibodies/dyes for 2 biomarkers as follows:
11. A viability dye (e.g. fixable live/dead, which can be obtained from Thermofisher) to exclude dead cells
12. Anti-CD3, to identify T cells.

These are general purpose biomarkers of which the skilled person is aware. An alternative to using CD3 is available to the skilled person which could be to use a T cell enrichment or purification kit and magnetic selection including a CD3 or CD4 or CD8 negative or positive selection kit containing a cocktail of biotinylated or otherwise conjugated antibodies specific to CD3 or CD4 or CD8 or multiple biomarkers on blood cells excluding CD3, CD4 or CD8. Such kits may also be used in flow cytometry, when co-stained with streptavidin or secondary antibodies containing fluorophores.

It will be understood that the term “T cell differentiation index”/“TEDI”, as used herein, provides a means to represent the data obtained herein to aid in interpretation of the scope of the invention and is not intended to limit the scope of the invention. The person skilled in the art will understand that such a methodology is an in-house term that is specific to the inventors and that various methods and techniques discussed herein can be performed using a variety of different apparatus, assay conditions, hardware and software components that may result in different numerical values obtained, but which equate to the same biological results to the examples provided herein; that the ratio of exhausted/activated T cells to naïve/resting T cells is higher in subjects with progressive, pre-cancerous lesion or nodule or in the presence of a malignant tumour relative to a subject with an indolent or regressive or non-progressing or low grade disease or no disease or a benign solid tumour.

The term “T cell differentiation index”/“TEDI”, as used herein, generally refers to the ratio of activated and/or exhausted T cells:naïve and/or resting T cells within a subject which can be represented numerically in the form of an index when a subject's results are compared against the average ratio of activated and/or exhausted T cells:naïve and/or resting T cells obtained from a plurality of patients known to have progressing or high-grade pre-invasive lesions or known to have a cancer to infer the risk that the subject has a progressing or high-grade pre-invasive lesion or nodules or having a solid malignant tumour. A subject's results may also be compared against their own previously determined ratio to indicate whether that subject is at risk of having a progressing or high-grade pre-invasive lesion or nodules or having a solid malignant tumour.

The methods of the present invention are performed on a blood sample obtained from a subject and hence occur in vitro. The methods can use whole blood. The methods can use PBMCs.

The present invention also provides a kit comprising a set of lyophilised antibodies or a fragment thereof, comprising antibodies binding to (i) CD39 and Ki67, and/or (ii) FoxP3 (optionally in combination with CD4). Such a kit may be used in or to perform the methods of the present invention. In embodiments, the kit may comprise one or more further lyophilised antibodies selected from antibodies binding to one or more biomarkers selected from CD45RA, CCR7, PD-1, CD57 or CD38. The kit may be used in a method for determining whether a subject is at risk for having a progressing or high-grade pre-invasive lesion or nodule or having a solid tumour.

The present invention also provides a device comprising means for receiving a sample of blood and contacting the sample with a set of lyophilised antibodies or a fragment thereof, comprising antibodies binding to (i) CD39 and Ki67 and/or (ii) FoxP3 (optionally in combination with CD4). Such a device may be used in or to perform the methods of the present invention. The device may be used in a method for determining whether a subject is at risk for having a progressing or high-grade pre-invasive lesion or nodule or having a solid tumour.

It will be understood that the present invention may be of use in providing information on whether a subject is at risk for having a progressing or high-grade pre-invasive lesion or nodule or having a solid malignant tumour. The methods of the present invention can also be implemented for valuable healthcare outcomes, examples by way of illustration, are set out below.

### A) Multi-Cancer Screening

At risk populations for any cancer to receive blood test (e.g. those with hereditary predispositions due to germline mutations). A positive result would trigger a confirmatory second test. A positive second test then triggers clinical follow up for relevant cancer type. The invention therefore provides a valuable tool which works on a variety of cancers, even when they come from different causes.

### B) Screening for Lung Cancer or Pre-Invasive Lung Neoplasia.

At-risk populations such as smokers or ex-smokers over 50 years of age to receive blood tests. A positive result would trigger a confirmatory second test. A positive second test then triggers sputum cytology, bronchoscopy and LDCT screen.

### c) Screening for Renal Cell Carcinoma.

At risk populations to receive blood test. A positive result would trigger a confirmatory second test. A positive second test then triggers clinical follow up including ultrasound, MRI X-ray or CT scan.

### D) Personalised Dynamic Immune Monitoring

An initial negative test is used to calibrate a personalised baseline value. A statistically significant increase in ratio of activated and/or exhausted T cells:naïve and/or resting T cells in any future test triggers a follow up test, where a positive result triggers further clinical evaluation for cancer types relevant to the demographic/subject.

### E) Healthy Adult Population Level Screening (Mass Testing)

Adults above the age of 40 can be screened, a positive result would trigger a confirmatory second test. A positive second test then triggers broad clinical follow up to screen for cancers of major incidence in the demographic. A negative test is used to establish an individual's personalised baseline.

### F) Detection of Cancer Recurrence

Subjects with a history of cancer may be screened. Scores obtained with methods of the present invention decrease post resection, suggesting that timepoints early after tumour removal/eradication may be suitable to establish a baseline. Statistically significant increases trigger a second test and/or clinical follow up. This would be to detect either minimal residual disease and or recurrence.

### G) Inclusion in Standard Clinical Blood Work

The invention disclosed herein could be incorporated into standard clinical blood work alongside tests such as ESR/CRP/white blood cell count or full blood count.

### Multi-Omic Analysis

Recent advances in technology have permitted integration of different data types in analytical strategies referred to as multi-modal or multi-omics. In cancer diagnostics combinations of multiple metrics from one or more platform have been shown to improve both specificity and sensitivity. For example, the integration of imaging data from CT scans with pathology assessed histological subtype and stage definition yields improved diagnostic and prognostic evaluation.

The method of the present invention may include analysing the proportion of T cells in the sample of blood obtained from the subject which are activated and/or exhausted comprises analysing a plurality of traits of the T cells and or using a plurality of T cell analysis techniques.

Combining data from more than one technique for the analysis of T cells in a sample of blood obtained from a subject may provide a further improved method for determining whether the subject is at risk for having a progressing or high-grade pre-invasive lesion, nodule or small mass, or having a solid malignant tumour (e.g., a stage I solid malignant tumour). Combining, data from a plurality of T cell analyses can provide improved specificity and sensitivity in early cancer detection. Combining, data from a plurality of T cell analyses can provide improved diagnostic and/or prognostic results.

Therefore the present invention provides a method for determining whether a subject is at risk for having a progressing or high-grade pre-invasive lesion, nodule or small mass, or having a solid malignant tumour (e.g., a stage I solid malignant tumour), the method comprising analysing the proportion of T cells in a sample of blood obtained from the subject which are activated and/or exhausted T cells by performing a plurality of T cell analyses.

A plurality means more than one. A plurality may also mean two or more.

In preferred embodiments, the plurality of T cell analyses comprises using cytometry (preferably flow cytometry, e.g., using a method as described elsewhere herein), and TCR-seq. In some embodiments, The T cell analyses may comprise (i) determining the diversity and/or clonality of T cell receptors by TCR Seq and (ii) determining biomarkers expressed by T cells by cytometry, preferably flow cytometry.

Therefore, the present invention also provides a method of determining whether a subject is at risk for having a progressing or high-grade pre-invasive lesion, nodule or small mass, or having a solid malignant tumour, using a sample of blood obtained from the subject, the method combining two or more T cell analyses comprising a) (i) measuring the ratio of activated and/or exhausted T cells:naïve and/or resting T cells determined by flow cytometry, or (ii) measuring the proportion of activated and/or exhausted cells as a percentage of T cells determined by flow cytometry and b) the diversity or clonality of the blood TCR repertoire.

The present invention also provides a method of determining whether a subject is at risk for having a progressing or high-grade pre-invasive lesion, nodule or small mass, or having a solid malignant tumour, the method comprising analysing the proportion of T cells in a sample of blood obtained from the subject which are activated and/or exhausted T cells by analysing a trait of the T cells, wherein the trait is a phenotypic trait which is
a) the diversity or clonality of T cell receptors on the T cells in the sample of blood, and
b) biomarker expression by the T cells in the sample of blood, optionally using cytometry.

The present invention is the first T cell focused multi-omics machine learning method for early cancer detection.

The inventors have shown that individual measures of blood T cells can help to detect malignant tumours or high grade preinvasive disease. Namely
i) the ratio of activated and/or exhausted T cells:naïve and/or resting T cells determined by flow cytometry, using key biomarkers Ki67 and CD39, and optionally one or more biomarkers selected from CD45RA, CCR7, CD38, PD1, or CD57, which the inventors refer to as the T cell early detection index (TEDI). Specifically, this ratio within CD4 T cells is a powerful classifier of patients with low vs high grade disease, or the proportion of activated and/or exhausted T cells as a percentage of T cells, using key biomarkers such as (i) FoxP3 and CD4, and/or (ii) CD39 and Ki67, and optionally one or more one or more biomarkers selected from CD45RA, CCR7, CD38, PD1, or CD57
ii) The diversity of the T cell repertoire, measured by TCR seq data, optionally the D50 index, which corresponds to the percentage of unique sequences that account for 50% of the total number of sequences, or the clonality of the T cell repertoire, measured by TCR seq data, optionally the a) repertoire of TCRs occupied by the top n clones (optionally wherein n is from 50-1000, for example, n=100) or b) the ratio of the repertoire of TCRs occupied by large clones:the repertoire of TCRs occupied by small clones

The inventors have further shown that coupling these metrics together may yield improved classifier performance to generate a multi-omics approach for enhanced cancer early detection.

The present invention therefore also provides a method for determining whether a subject is at risk for having a progressing or high-grade pre-invasive lesion, nodule or small mass, or having a solid malignant tumour, using a sample of blood obtained from the subject, the method combining two or more T cell analyses comprising a) measuring the ratio of activated and/or exhausted T cells:naïve and/or resting T cells determined by flow cytometry, or measuring the proportion of activated and/or exhausted cells as a percentage of T cells determined by flow cytometry and
b) the diversity or clonality of the blood TCR repertoire.

The present invention provides a method for determining whether a subject has a high grade or progressing preinvasive lesion or nodule or small mass or solid malignant tumour vs has no malignant tumour, or a benign tumour or a benign or non progressing or low grade lesion or nodule or small mass, using a sample of blood obtained from the subject, the method comprising combining two or more T cell analyses comprising:
measuring the ratio of activated and/or exhausted T cells:naïve and/or resting T cells determined by flow cytometry, and
the diversity or clonality of the blood TCR repertoire. Measures of the diversity or clonality of the blood TCR repertoire may include the D50 index.

The present invention also provides a method for determining whether a subject is at risk for having a progressing or high-grade pre-invasive lesion, nodule or small mass, or having a solid malignant tumour, using a sample of blood obtained from the subject, the method combining two or more T cell analyses comprising:
measuring the ratio of activated and/or exhausted T cells:naïve and/or resting T cells determined by flow cytometry, and
the diversity or clonality of the blood TCR repertoire. Measures of the diversity or clonality of the blood TCR repertoire may include the D50 index.

Combining data obtained from two different methods and/or the two or more T cell analyses may involve one or more of machine learning, artificial intelligence or mathematical modelling. In some embodiments and examples, the machine learning uses the XGBoost algorithm, e.g., using the methods described herein. However, other suitable algorithms may be used.

In preferred embodiments, the diversity or clonality of the blood TCR repertoire is determined by TCR-seq. The diversity or clonality of the blood TCR repertoire may be determined using one or more diversity or clonality metrics as described herein.

The measuring the ratio of activated and/or exhausted T cells:naïve and/or resting T cells determined by flow cytometry, or measuring the proportion of activated and/or exhausted cells as a percentage of T cells determined by flow cytometry may be as determined using any of the methods or embodiments of the methods described herein.

In some preferred embodiments, the activated and/or exhausted T cells express the biomarkers Ki67 and CD39.

In some preferred embodiments, the activated and/or exhausted T cells express the biomarker FoxP3, wherein the T cells are CD4+ T cells, optionally in combination with CD39. In some embodiments, the CD4+ T cells express FoxP3, CD39 and Ki67. In some embodiments, the CD4 T cells express FoxP3, CD39 and do not express CD45RA.

In some embodiments and examples, the diversity of the blood is measured using the D50 index. In other embodiments and examples, the diversity of the blood is measured using the Hill number. However, other methods of measuring the diversity of the TCR blood repertoire may be used.

In some embodiments and examples, the clonality of the blood is measured using the ratio of the repertoire of TCRs occupied by large clones:the repertoire of TCRs occupied by small clones. This may be determined using the method disclosed herein.

### Methods of Treatment

Also disclosed herein is a method of treating a subject determined to be at risk for having a progressing or high-grade pre-invasive lesion, nodule or small mass, or having a solid malignant tumour, comprising analysing the proportion of T cells in a sample of blood obtained from the subject which are activated and/or exhausted T cells by analysing a trait of the T cells, and treating the subject determined to be at risk. The analysing may be as described elsewhere herein. The subject may be determined to be at risk using any method described elsewhere herein.

Also disclosed herein is a method of treating a subject determined to be at risk for having a progressing or high-grade pre-invasive lesion, nodule or small mass, or having a solid malignant tumour, comprising analysing the proportion of T cells in a sample of blood obtained from the subject which are activated and/or exhausted T cells by analysing a trait of the T cells, wherein the trait is a phenotypic trait and wherein the analysing comprises
a) analysing the proportion of T cells in a sample of blood obtained from the subject which are activated and/or exhausted T cells using biomarker expression, optionally using cytometry, wherein the subject is at risk if
(i) the ratio of activated and/or exhausted T cells:naïve and/or resting T cells is equal to or greater than a ratio of activated and/or exhausted T cells:naïve and/or resting T cells of a comparison subject, or in a plurality of comparison subjects, or
(ii) the ratio of activated and/or exhausted T cells:T cells which are not activated and/or exhausted is equal to or greater than a ratio of activated and/or exhausted T cells:T cells which are not activated and/or exhausted of a comparison subject, or in a plurality of comparison subjects.
(iii) the proportion of activated and/or exhausted T cells is higher than the proportion of activated and/or exhausted T cells in a comparison subject or a plurality of comparison subjects, and/or
b) determining the diversity or clonality of T cells in a sample of blood obtained from the subject, optionally using TCR-seq, wherein the subject is at risk if
(i) the diversity of the repertoire of TCRs is lower than the diversity of the repertoire of TCRs of a comparison subject or a plurality of comparison subjects,
ii) D50 diversity score is lower than a D50 diversity score obtained from analysing T cells in a sample of blood obtained from a comparison subject or a plurality of comparison subjects,
iii) a clonality score is higher than a clonality score obtained from analysing T cells in a sample of blood obtained from a comparison subject or a plurality of comparison subjects, or
iv) the Hill number is lower than a Hill number obtained from analysing T cells in a sample of blood obtained from a comparison subject or a plurality of comparison subjects, or
v) the clonality of the repertoire of TCRs occupied by the top n clones is higher than the clonality of the repertoire of TCRs occupied by the top n clones in a comparison subject or a plurality of comparison subjects, optionally wherein n=100, or
vi) the proportion of the repertoire of TCRs occupied by small clones is lower than the proportion of the repertoire of TCRs occupied by small clones in a comparison subject or a plurality of comparison subjects, or
vii) the proportion of the repertoire of TCRs occupied by large clones is higher than the proportion of the repertoire of TCRs occupied by large clones in a comparison subject or a plurality of comparison subjects or
viii) the ratio of the repertoire of TCRs occupied by large clones:small clones is higher than the ratio of the repertoire of TCRs occupied by large clones:small clones in a comparison subject or a plurality of comparison subjects, optionally wherein the comparison subject is a healthy subject, or the plurality of comparison subjects are healthy subjects, and
treating the subject if the subject is at risk.

In some embodiments, the treating may comprise administering an anti-cancer therapeutic. In some embodiments, the treating may comprise administering a therapeutic suitable for treating pre-invasive neoplasia and/or a high grade pre-invasive lesion, nodule or small mass. In some embodiments, the treating may comprise administering a therapeutic suitable for treating a solid malignant tumour, for example, a stage I solid malignant tumour.

Also disclosed herein is an anti-cancer therapeutic for use in a method of treatment of a subject determined to be at risk for having a progressing or high-grade pre-invasive lesion, nodule or small mass, or having a solid malignant tumour, comprising analysing the proportion of T cells in a sample of blood obtained from the subject which are activated and/or exhausted T cells by analysing a trait of the T cells, wherein the anti-cancer therapeutic is administered to the subject determined to be at risk. The analysing may be as described elsewhere herein. The subject may be determined to be at risk using any method described elsewhere herein.

In some embodiments, the analysing comprises analysing a trait of the T cells, wherein the trait is a phenotypic trait and wherein the analysing comprises
a) analysing the proportion of T cells in a sample of blood obtained from the subject which are activated and/or exhausted T cells using biomarker expression, optionally using cytometry, wherein the subject is at risk if
(i) the ratio of activated and/or exhausted T cells:naïve and/or resting T cells is equal to or greater than a ratio of activated and/or exhausted T cells:naïve and/or resting T cells of a comparison subject, or in a plurality of comparison subjects, or
(ii) the ratio of activated and/or exhausted T cells:T cells which are not activated and/or exhausted is equal to or greater than a ratio of activated and/or exhausted T cells:T cells which are not activated and/or exhausted of a comparison subject, or in a plurality of comparison subjects.
(iii) the proportion of activated and/or exhausted T cells is higher than the proportion of activated and/or exhausted T cells in a comparison subject or a plurality of comparison subjects, and/or
b) determining the diversity or clonality of T cells in a sample of blood obtained from the subject, optionally using TCR-seq,

### In Some Embodiments, the Subject is at Risk if

(i) the diversity of the repertoire of TCRs is lower than the diversity of the repertoire of TCRs of a comparison subject or a plurality of comparison subjects,
ii) D50 diversity score is lower than a D50 diversity score obtained from analysing T cells in a sample of blood obtained from a comparison subject or a plurality of comparison subjects,
iii) a clonality score is higher than a clonality score obtained from analysing T cells in a sample of blood obtained from a comparison subject or a plurality of comparison subjects, or
iv) the Hill number is lower than a Hill number obtained from analysing T cells in a sample of blood obtained from a comparison subject or a plurality of comparison subjects, or
v) the clonality of the repertoire of TCRs occupied by the top n clones is higher than the clonality of the repertoire of TCRs occupied by the top n clones in a comparison subject or a plurality of comparison subjects, optionally wherein n=100, or
vi) the proportion of the repertoire of TCRs occupied by small clones is lower than the proportion of the repertoire of TCRs occupied by small clones in a comparison subject or a plurality of comparison subjects, or
vii) the proportion of the repertoire of TCRs occupied by large clones is higher than the proportion of the repertoire of TCRs occupied by large clones in a comparison subject or a plurality of comparison subjects or
viii) the ratio of the repertoire of TCRs occupied by large clones:small clones is higher than the ratio of the repertoire of TCRs occupied by large clones:small clones in a comparison subject or a plurality of comparison subjects, optionally wherein the comparison subject is a healthy subject, or the plurality of comparison subjects are healthy subjects.

### EXAMPLES SECTION

In this section, Examples are described for analysing T cells isolated from a subject. The methods in the Examples provided herein for analysing T cells isolated from a subject can be implemented using a variety of methodologies, including using a variety of apparatus, hardware and software components that include (but are not limited to) different cytometry devices, assay conditions, computational hardware and analysis software. It will be understood to the person skilled in the art that in general, the methods provided herein in the Examples are merely an example. The person skilled in the art will know that that the various steps and techniques discussed herein can be performed using a variety of different apparatus, assay conditions, computational hardware and software components that may result in different numerical values obtained, but which equate to the same biological results to the Examples provided herein. It will be understood to the person skilled in the art that in general, the methodologies and results provided herein in the below Examples are not intended to limit the scope of the invention.

TABLE 1
List of antibodies used in Examples 1-3
Samples were acquired on FACSymphony A5 High-Parameter
Cell Analyser from BID Biosciences.
Phenotyping Panel: Fluorochromes and Antibodies
(Biomarkers are surface biomarkers unless otherwise stated)

| Channel | Target | Clone | Supplier | Cat# |
|---|---|---|---|---|
| BUV395 | Ki67 | B56 | BD Biosciences | 564071 |
| | (intracellular biomarker) | | | |
| BUV496 | CD8 | RPA-T8 | BD Biosciences | 741199 |
| BUV563 | CD45RA | HI100 | BD Biosciences | 612926 |
| BUV615 | CD4 | OKT4 | BD Biosciences | 750975 |
| BUV661 | CD101 | V7.1 | BD Biosciences | 750530 |
| BUV737 | CD38 | HB-7 | BD Biosciences | 741902 |
| BUV805 | CD103 | Ber-ACT-8 | BD Biosciences | 748501 |
| BV421 | FOXP3 | 206D | Biolegend | 320124 |
| | (intracellular biomarker) | | | |
| BV480 | Live/Dead Fixable Aqua | N/A | ThermoFisher | L34966 |
| BV605 | CD57 | QA17A04 | Biolegend | 393304 |
| BV650 | CCR7 | G043H7 | Biolegend | 353234 |
| | (chemokine receptor) | | | |
| BV711 | CD39 | A1 | Biolegend | 328228 |
| BV750 | CD27 | O323 | Biolegend | 302850 |
| BV785 | TIM3 | F38-2E2 | Biolegend | 345032 |
| BB515 | PD-1 | EH12.1 | BD Biosciences | 594494 |
| PerCP-eFluor | EOMES | WD1928 | eBioscience | 46-4877-42 |
| 710 | (intracellular biomarker) | | | |
| PE | TCF7 | 7F11A10 | Biolegend | 655208 |
| | (intracellular biomarker) | | | |
| PE-CF594 | Granzyme B | GB11 | BD Biosciences | 562462 |
| | (intracellular biomarker) | | | |
| PE-Cy5 | CXCR4 | 12G5 | Biolegend | 306508 |
| | (chemokine receptor) | | | |
| PE-Cy7 | TIGIT | A15153G | Biolegend | 372714 |
| APC | Tox | REA473 | Miltenyi Biotec | 130-120-716 |
| | (intracellular marker) | | | |
| APC-R700 | HLA-DR | G46-6 | Biolegend | 565127 |
| APC-Fire | CD3 | UCHT1 | Biolegend | 300470 |

### An Example Protocol for Processing of Blood Samples for Flow Cytometry Analysis

Blood samples were collected in Vacutainer EDTA blood collection tubes (BD), PBMCs isolated by gradient centrifugation (750 g for 10 minutes) on Ficoll Paque Plus (GE Healthcare). The interface was washed twice with complete RPMI-1640, resuspended in 90% FBS with 10% DMSO (Sigma) and cryopreserved in liquid nitrogen or in a −180 degrees Celsius freezer system prior to staining. To characterise T cell differentiation profiles of HG vs LG samples cryopreserved PBMCs from were thawed with warm R20 media (RPMI with 20% FBS, L-glutamine, HEPES and Penicillin/Streptomycin) and washed with R10 media containing DNase I grade II 37.5 μg/mL (Roche, 10104159001). The samples were subjected to 20 mins incubation of Live/Dead Fixable Aqua at RT in the dark. Samples were then washed in PBS and resuspended with FACS buffer (PBS+2% FBS+2 mM EDTA) and plated on a 96-well U bottom plate containing Fc Receptor Binding Inhibitor Polyclonal Antibody (Thermo Fisher, 14-9161-73) and surface antibodies for chemokine receptors (CXCR4, CCR7) at room temperature (RT) for 15 mins, followed by another 30 mins incubation with all additional surface antibodies, on ice. Plates were washed twice with FACS buffer and fixed with Foxp3 Transcription Factor Fixation/Permeabilization Concentrate and Diluent solutions (Thermo Fisher, 00-5521-00) for 30 mins at RT. Cells were washed twice with X1 Permeabilization Buffer (Thermo Fisher, 00-8333) followed by 1 hour incubation with a cocktail of intracellular antibodies at RT. The plates were washed 3 times and resuspended with X1 Permeabilization Buffer before sample acquisition using FACSymphony (BD Biosciences.

### An Example Protocol for Acquisition and Processing

Samples were acquired using a BD Symphony cytometer according to the manufacturer's instructions using the BD FACS DIVA software. Raw FCS files were exported and files imported into FlowJo. Compensation matrices were calculated in FACS DIVA from single-stain compensation bead controls and optimised in FlowJo. To do so, files from each batch (two in total from consecutive days) were concatenated separately as a reference file and optimised matrices applied to each batch. Dead cells and compensation artefacts were excluded as described previously (NC) and 5000 live CD3+CD4+ or 2000 CD3+CD8+ events were down-sampled and exported per file.

### An Example Protocol for Clustering

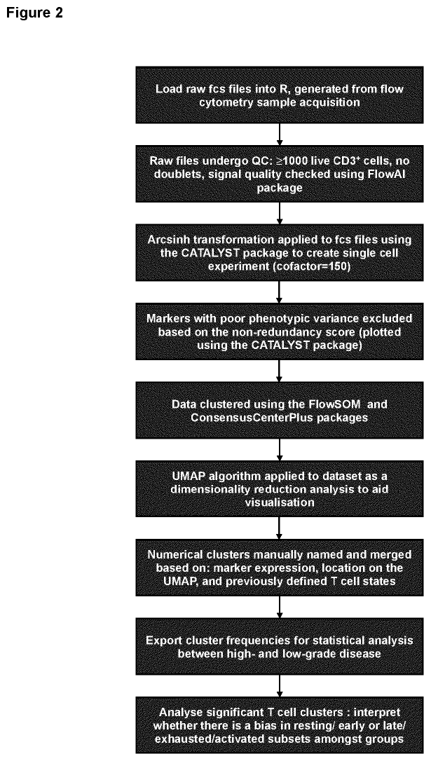

FIG. 2 shows a workflow showing the protocol of how the clustering of the flow cytometry data was completed following the analysis steps outlined.

Only samples with minimum 1000 live CD3+ cells were analysed. FCS files underwent quality control of signal acquisition, assessed by the FlowAI package (v1.24). An Arcsinh transformation was applied to the data, using the prepData function from the CATALYST package (v1.18.1), with the cofactor set to 150. Any biomarkers with a poor contribution to phenotypic variance were excluded pre-clustering. These were determined using the PCA-based non-redundancy score (NRS) as described in the Nowicka et al. pipeline (Nowicka M, Krieg C, Crowell H L et al. CyTOF workflow: differential discovery in high-throughput high-dimensional cytometry datasets [version 4; peer review: 2 approved]. F1000Research 2019, 6:748 (https://doi.org/10.12688/f1000research. 11622.4). In the initial analysis (e.g., of the pre-invasive data) FoxP3, CD103, Tim3, CXCR4, TIGIT, were all excluded from clustering (live/dead, CD3, CD8 and CD4 were excluded as FCS files only included cells pre-gated on live CD4 or CD8 T cells). In subsequent analysis, FoxP3 was included in the clustering panel.

Data was clustered using the FlowSOM package (v2.2.0) onto a 15×15 node square self-organising map (SOM). Nodes were clustered by the ConsensusCenterPlus package (v1.58.0), as described by Nowicka et al. The UMAP algorithm was applied as a dimension reduction analysis to better understand the phenotypic relevance between individual clusters. The resulting clusters were manually merged based on biomarker expression similarity, occupation of space on the UMAP, and previously defined T cell states. 16 clusters of CD4 T cells and 15 clusters of CD8 T cells were resolved. All computational packages were sourced from https://cran.r-project.org/and https://www.bioconductor.org/.

Table 2: A List of T Cell Clusters that were Differentially Abundant Between High Grade or Low Grade Pre-Invasive Lesion or Nodule.

Clusters found to be enriched in high-grade were found to express biomarkers of activation and late differentiation, proliferation or exhaustion, in particular the biomarkers CD39, known to be expressed on tumour specific T cells, and the marker Ki67 which denotes that cells are actively proliferating. TEDI scores were generated by dividing high-grade-associated clusters by the low grade associated clusters within each lineage (e.g. sum of high grade clusters/sum of low grade clusters* for CD4=CD4.TEDI) to develop a metric that would have the highest discriminatory power, which reflects the state of differentiation within that lineage. The same was completed for CD8 T cells and all T cells combining CD4 and CD8.

Unsupervised clustering refers to using computational analysis to determine which type of T cell is present in a given sample. This is a preferred alternative for initial analysis of flow cytometry data to ‘supervised’ analysis such as manually gating cells in a sample using prior knowledge and selecting specific marker combinations.

| | | | | | Which | |
|---|---|---|---|---|---|---|
| | | | | | grade is | |
| | | | | Biological | the cluster | |
| Subset | Full name | Lineage | Biomarkers | relevance | higher in? | Identified by |
| Tex.Prolif | Exhausted, | CD8 | PD-1, Ki67, | Exhaustion, | High grade | Unsupervised |
| | proliferating | | CD39 | activation | | clustering |
| | CD8 T cell | | | | | |
| TEMRA.Act | Terminally | CD8 | CD39, Ki67 | Terminal | High grade | Unsupervised |
| | differentiated effector | | CD45RA, | differentiation, | | clustering |
| | memory cells (RA) | | CD57 | activation | | |
| | that are activated | | | | | |
| Naive | Naïve CD8 T | CD8 | CD45RA, | Resting, | Low grade | Unsupervised |
| | cells | | CCR7 | unstimulated, | | clustering |
| | | | | non-antigen | | |
| | | | | experienced | | |
| TEMRA.Rest | Terminally | CD8 | CD4RA | Resting | Low grade | Unsupervised |
| | differentiated effector | | CCR7− | terminally | | clustering |
| | memory cells (RA) | | CD57− | differentiated | | |
| | that are resting | | | memory T cells | | |
| Treg.prolif.PD-1 | Regulatory T cells | CD4 | CD39, Ki67, | Antigen- | High grade | Unsupervised |
| | that are proliferating | | PD-1, | Activated | | clustering |
| | and express PD-1 | | | regulatory T cells | | |
| TEM | Effector memory | CD4 | CD39, Ki67, | Effector | High grade | Unsupervised |
| | CD4 T cells | | CCR7− | memory CD4 | | clustering |
| | | | CD45RA− | T cells | | |
| Treg | Regulatory T | CD4 | CD39, Ki67, | Regulatory T | High grade | Unsupervised |
| | cells | | | cells suppress | | clustering |
| | | | | immunity and | | |
| | | | | play a role in | | |
| | | | | cancer promotion | | |
| TPEX | Progenitor | CD4 | CD39, Ki67, | Early version | High grade | Unsupervised |
| | exhausted | | PD-1 | of exhausted | | clustering |
| | CD4 T cells | | | CD4 T cells | | |
| Treg.prolif | Proliferating | CD4 | CD39, Ki67hi, | Proliferating | High grade | Unsupervised |
| | regulatory T | | PD-1 | regulatory T | | clustering |
| | cells | | | cells | | |
| Tem.Prolif | Proliferating | CD4 | CD39, Ki67hi, | Activated, | High grade | Unsupervised |
| | effector memory | | CCR7− | dividing late | | clustering |
| | CD4 T cells | | CD45RA− | differentiated | | |
| | | | | effector | | |
| | | | | memory cells | | |
| TCM.CD38 | Central memory | CD4 | CD39, Ki67hi, | An early | High grade | Unsupervised |
| | CD4 T cells | | CD38, CCR7, | activated T cell | | clustering |
| | expressing CD38 | | CD45A | subset | | |
| Tex.Prolif | Exhausted, | CD4 | CD39, | Exhaustion, | High grade | Unsupervised |
| | proliferating | | Ki67hi, | activation | | clustering |
| | CD8 T cell | | PD-1 | | | |
| Cytolytic | Cytolytic | CD4 | CD57 | Terminally | High grade | Unsupervised |
| | CD4 T cells | | | differentiated | | clustering |
| | | | | and cytotoxic | | |
| | | | | CD4 T cells | | |
| Naïve.TCF7 | Naïve CD4 T cells | CD4 | CD45RA+ | A subset of | Low grade | Unsupervised |
| neg | that have lost | | CCR7+ | naïve T cell | | clustering |
| | TCF7 expression | | CD27+ | | | |
| | | | TCF7− | | | |
| TCM.rest | Resting | CD4 | CD45RA− | Early | Low grade | Unsupervised |
| | central | | CCR7+ | differentiated | | clustering |
| | memory | | CD38− | resting | | |
| | CD4 T cells | | | memory cells | | |
| | | | | that are | | |
| | | | | unstimulated | | |
| Naïve | Naïve T cells | CD4 | CD45RA+ | Naïve, | Low grade | Unsupervised |
| | | | CCR7+ | unstimulated T | | clustering |
| | | | | cells | | |

### Example 1

In Example 1, the inventors used the example protocols set out above.

TABLE 3
Clinical variables of patients used to generate data in Example 1.

Total

| Patients | 30 | | | |
|---|---|---|---|---|
| Samples | 69 | | Mean 2.3 (1-6) | |
| Age (mean) | 68.5 | | (52-89) | |
| Smoking status | | | | |
| Never | 2 | patients | 3 | samples |
| Ex | 19 | patients | 42 | samples |
| Current | 9 | patients | 24 | samples |
| Pack years (mean) | 46.4 | | (0-120) | |
| Grade | | | | |
| High | 14 | patients | 34 | samples |
| Low | 16 | patients | 35 | samples |

### Analysis of Cluster Distribution in High Versus Low Grade Samples

The frequency of each cluster was expressed as a % of parent (CD4 or CD8) for each of the 66 samples and Mann-Whitney tests were performed in R to compare average cluster frequency between high-grade (HG) vs low-grade (LG) samples for each cluster. Separate rounds of analysis focused on CD4 and CD8 T cell subsets were performed, with multiple correction adjustment via benjamini-hochberg, applying a false discovery rate of 0.05. To account for multiple samples being drawn from individual patients, analysis was repeated using the mean frequency of a given cluster in each patient, using all available samples, yielding similar results.

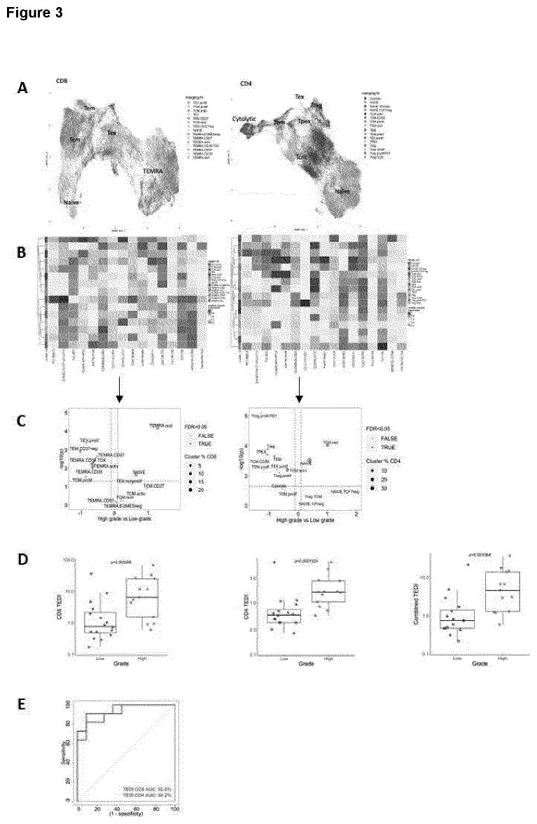

12 (of 16) clusters of CD4 T cell clusters and 4 (of 15) CD8 T cell clusters were significantly differentially abundant in HG vs LG (FDR<0.05 sample level analysis and p<0.05 in patient level analysis). The clusters enriched in HG patients represented late T cell differentiation, activation and exhaustion within both CD4 and CD8 T cells. This shows which clusters of T cells are present across all samples and their relative abundance. Thereby showing that T cells can be classified based on their phenotype into low-grade and high-grade disease. FIG. 3C. The presence of HG (vs LG) lesions is associated with a significantly increased frequency of activated, memory and exhausted CD8 and CD4 T cells and a loss of resting Tcm and naïve cells in the blood (FIG. 3A), This shows that there are multiple types of activated/exhausted and resting/naïve T cell present in the pool of samples. further demonstrated with significantly different expression of CD45RA, CCR7, Ki67, CD39, CD57 and CD38 biomarkers in activated and/or exhausted T cell types compared to naïve and/or resting T cell types (FIG. 3B-C). The ratio of [T cell clusters significantly enriched in high-grade disease samples:T cell clusters significantly enriched in low grade samples] for CD4 (AUC 94.2%) or CD8 (AUC 92.6%) T cells were able to help discriminate patients with HG lesions in ROC analysis, meaning that 92-94% of patients can be correctly classified as having High or Low-grade disease using the invention. FIG. 3D-E.

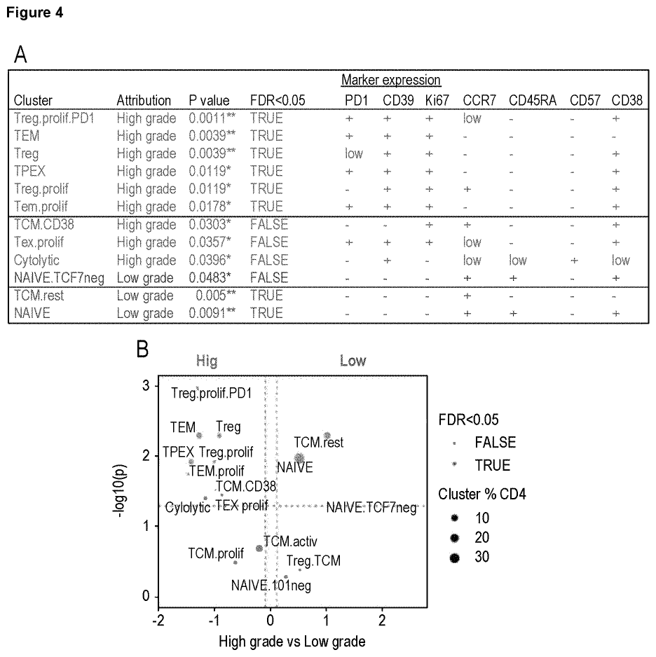

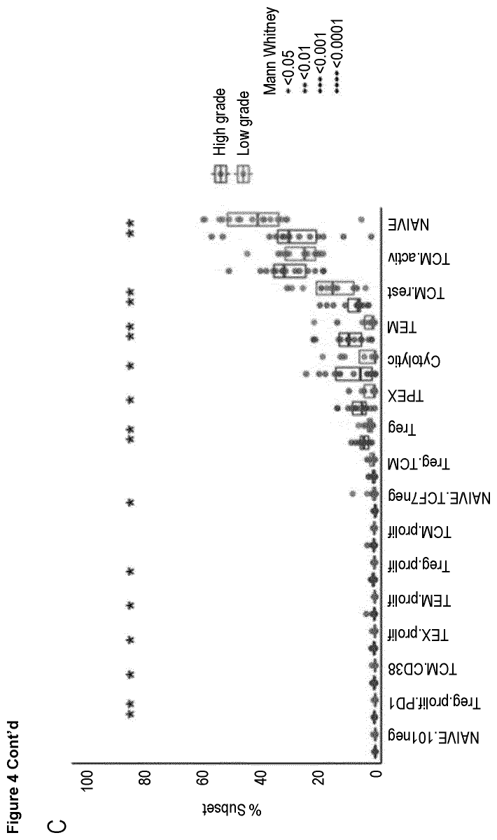

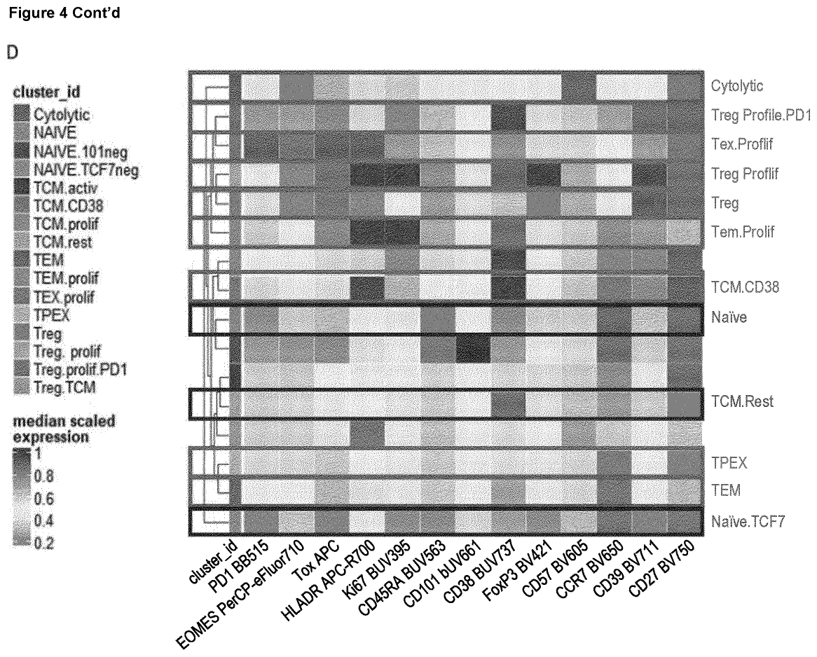

In CD4 T cells 8 of the 9 subsets significantly over-represented in patients with HG disease expressed at least one of the following biomarkers: PD-1 (a biomarker of antigen driven activation and exhaustion; 6/9 HG-clusters) CD39 (a biomarker of exhausted, regulatory and tumour reactive T cells; 8/9 HG-clusters) and Ki67 (an activation biomarker expressed on proliferating T cells; 8/9 HG-clusters). Moreover, CD39 and Ki67 co-expression defined 7 of the 9 T cell subsets enriched in HG disease. HG associated clusters of CD4 T cells also lacked CD45RA and 7 of 9 also lacked high levels of the early differentiation biomarker CCR7. In contrast, all 3 CD4 T cell clusters enriched in LG were devoid of the activation and exhaustion biomarkers PD-1, Ki67, and CD39 (i.e. PD-1-KI67-CD39−), and all 3/3 expressed CCR7 and 2/3 expressed CD45RA. This imbalance in CD4 T cell differentiation is indicative of increased antigen exposure with HG disease and suggests that HG vs LG patients can be distinguished using a combination of the biomarkers PD-1, Ki67, CD39, CCR7 and CD45RA. In addition, a single cluster of Cytolytic phenotype CD4 T cells enriched in HG disease that could be identified by unique expression of the terminal differentiation biomarker CD57. FIG. 4. This shows that expression of CD45RA, CCR7, Ki67, CD39, CD57 and CD38 biomarkers were significantly different in activated and/or exhausted T cell types compared to naïve and/or resting T cell types and that these biomarkers can be used to determine the ratio of activated and/or exhausted T cells compared to naïve and/or resting T cells in a subject, thereby distinguishing between high-grade and low-grade disease.

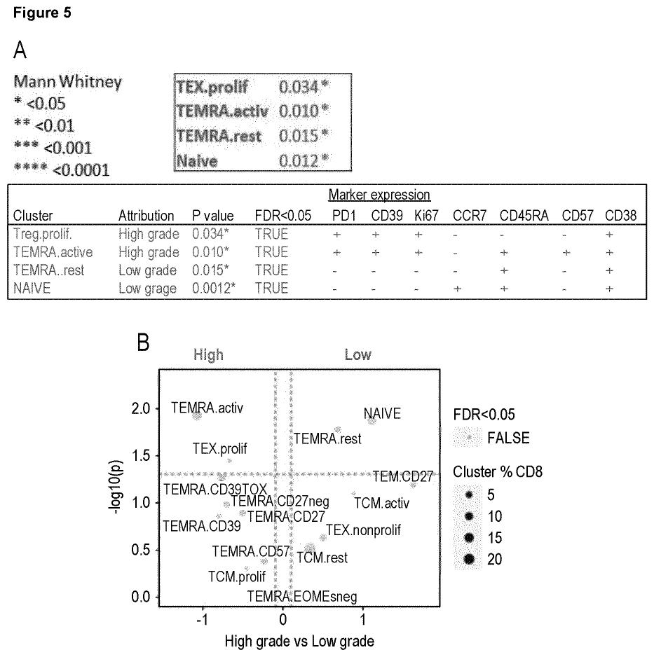

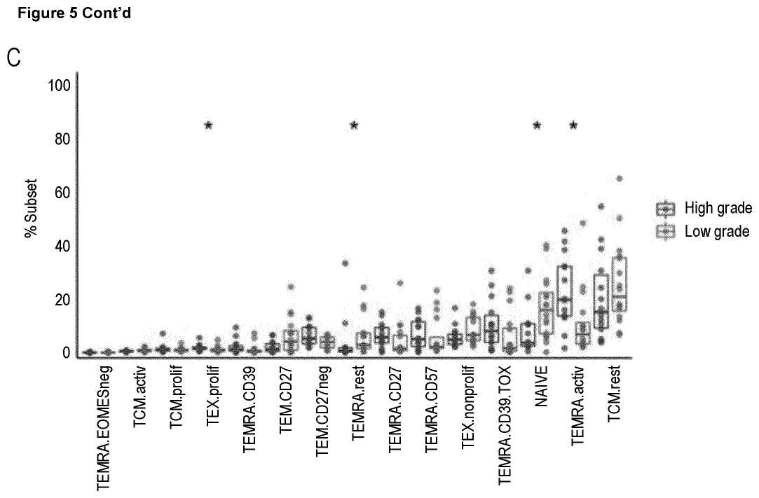

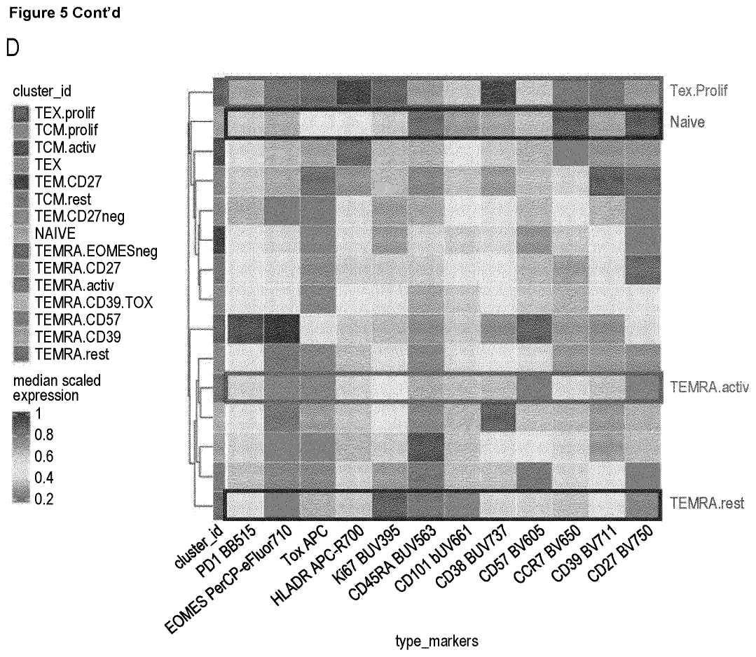

Skewing in T cell differentiation was also observed in CD8 T cells, where two clusters were enriched in HG disease that represent end points of late-stage differentiation and activation in the blood. These 2 clusters once again both co-expressed CD39 and Ki67, but could be distinguished as exhausted (PD-1 high) or terminally differentiated (CD57+CD45RA+) based on additional biomarkers. FIG. 5 As observed in CD4 T cells, CD8 T cell clusters enriched in LG disease were PD-1-CD39-Ki67-indicating a resting state. These clusters were naïve cells known to be enriched in less antigen experienced individuals, and resting memory cells. Identification of these 4 populations from all remaining CD8 T cells via clustering required the same 6 biomarkers as CD4 T cells; namely PD-1, Ki67, CD39, CCR7, CD45RA, CD57. In addition, the biomarker CD38 was required to identify the activated, terminally differentiated population. FIG. 5. This shows that expression of CD45RA, CCR7, Ki67, CD39, CD57 and CD38 biomarkers were significantly different in activated and/or exhausted T cell types compared to naïve and/or resting T cell types and that these biomarkers can be used to determine the ratio of activated and/or exhausted T cells compared to naïve and/or resting T cells in a subject, thereby distinguishing between high-grade and low-grade disease. This data therefore supports measuring systemic T cell differentiation as an innovative strategy for early detection. Specifically, the data show that measuring the ratio of activated, exhausted and late memory T cell subsets:Naïve, resting and earlier differentiated T cells is a novel metric that distinguishes individuals with less pathogenic lesions (LG) from patients with lesions more likely to progress to NSCLC (HG).

### Generation of an Example of a T Cell Early Detection Index (TEDI)

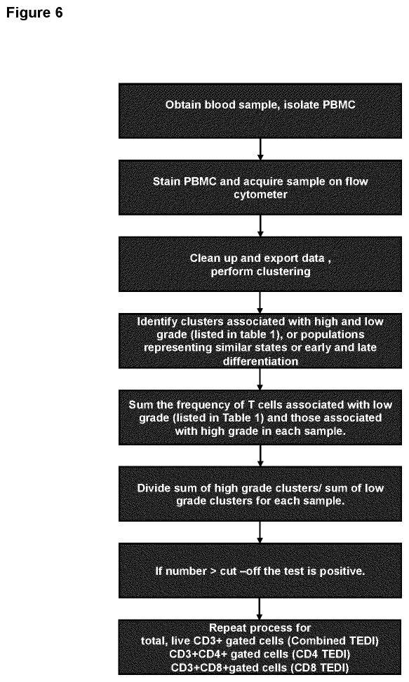

To simplify these results, the change in CD4 or CD8 clusters was converted to a single score referred to as the T cell early detection index (TEDI). To generate TEDI a ratio of [sum freq. of all CD4 T cell clusters enriched in HG disease]/[sum freq. of all clusters enriched in LG disease] was calculated and the process repeated for CD8 T cells, a simple workflow is shown in FIG. 6

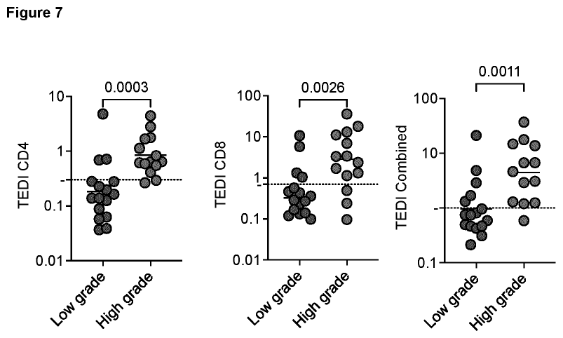

In addition, the mean average of CD4 and CD8 T cell TEDI scores was calculated to generate a Combined TEDI score. Receiver operator characteristic (ROC) curves and calculated the areas under the curve (AUC) were generated for each TEDI score, shown in FIG. 7. This Figure summarises the key results from Example 1, showing that the ratio of exhausted/activated:naïve resting T cells is significantly higher in high vs low grade disease. The same data is also shown in FIG. 3C

Table 4: Sensitivity and Specificity Values with their Corresponding Threshold Probabilities for ROC Analysis

To determine the optimum TEDI cut-off values from the ROC analysis, all possible sensitivity and specificity values with their corresponding threshold probabilities were calculated, as ROC curves represent a trade-off between the true and false positive rates. The threshold probability that yielded the highest combined sensitivity and specificity was selected, known as the Youden Index, a commonly used method to estimate the optimal cut-off point for a diagnostic Fluss R, Faraggi D, Reiser B. Estimation of the Youden Index and its associated cutoff point. Biom J. 2005 August; 47 (4): 458-72. doi: 10.1002/bimj.200410135. PMID: 16161804.

The Youden-determined cut-offs were adjusted to prioritise sensitivity (reducing false negatives) over specificity (increasing false positive), yielding threshold probabilities of the TEDI score required for correct high-grade patient classification, with the greatest accuracy. These adjusted cut off values represent the values at which a test using the TEDI indices would be classified as positive or negative.

| | | | | | Sensitivity |
|---|---|---|---|---|---|
| | | | | Cut-off | Adjusted |
| TEDI | AUC (%) | Sensitivity | Specificity | value | Cut-off |
| CD4 | 94.2 | 0.91 | 0.91 | 0.7054743 | 0.3 |
| | (85.05-100) | (0.73-1) | (0.73-1) | | |
| CD8 | 92.6 | 0.82 | 0.91 | 1.49561 | 0.6 |
| | (81.92-100) | (0.55-1) | (0.73-1) | | |
| Combined | 93.4 | 0.91 | 0.91 | 1.086619 | 1 |
| | (82.6-100) | (0.73-1) | (0.73-1) | | |

### Manual Gating

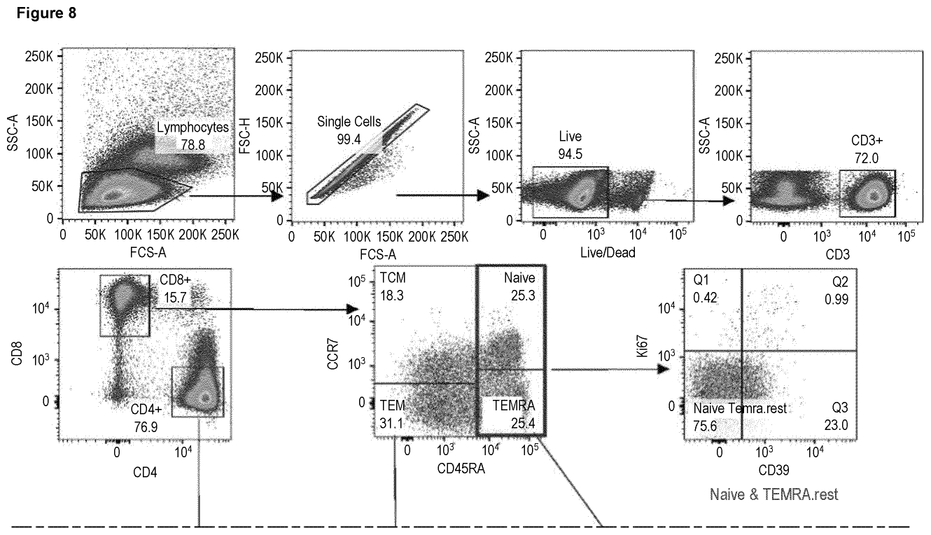

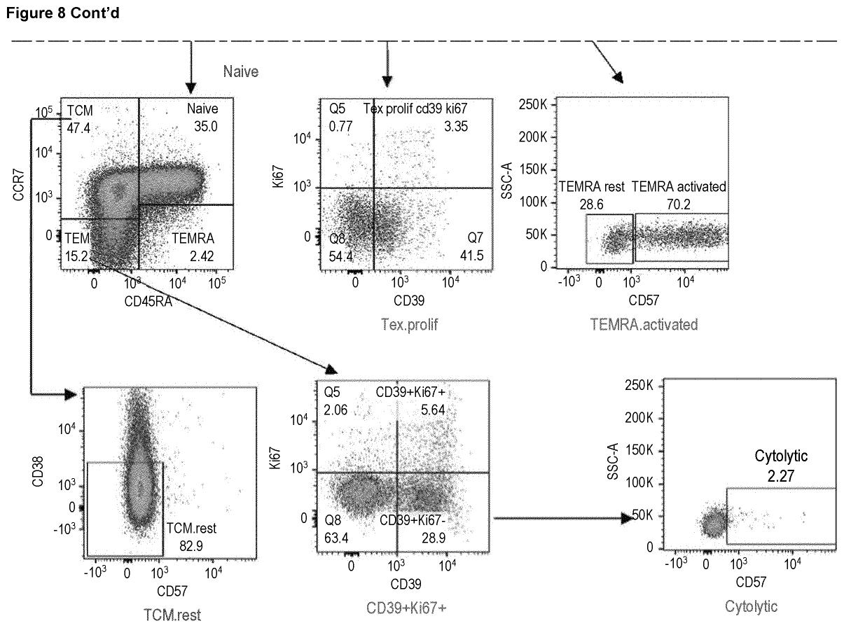

The FlowJo software (BD) was used to manually gate the computationally resolved T cell clusters of interest (those significantly enriched in HG or LG samples after using the mean frequency of each patient's samples). FIG. 8 This shows the person skilled in the art could bypass computational analysis to identify several activated/exhausted and naïve/resting T cell clusters manually using compensated FCS (Flow Cytometry Standard) files in FlowJo software. In this Example, this step is not only necessary to validate output of the unsupervised clustering analysis but also to ensure that equivalent results can be generated with a minimal panel of antibodies. This demonstrates that standard methodologies can be used that do not require computational expertise, development of a TEDI or other similar forms of statistical indexing/classification. This means that a subject's population of T cells can be analysed and where the ratio of that subject's T cells show more activated and/or exhausted T cell's compared to naïve/resting T cells based on detection of a combination of biomarker expression, a subject can be deemed at risk of having a progressing or high-grade pre-invasive lesion or nodule or having a solid tumour. A subject can then be administered to further testing, thereby simplifying clinical implementation. Such standard methodology can be applied to a point of care device (not shown) where a subject applies a blood sample to the device containing a panel of antibody biomarkers for analysis with the subject's T cells and where the combination of biomarkers present on the subject's T cells will provide an indication whether the subject is at risk of having a progressing or high-grade pre-invasive lesion or nodule or having a solid tumour. In preliminary analysis of the pre-invasive data, the results showed that a panel of CD45RA, CCR7, Ki67, CD39, CD57 and CD38 biomarkers can be applied to manually gate the clusters of CD8 and CD4 T cells that were significantly different in high-grade or low-grade disease. FIG. 8.

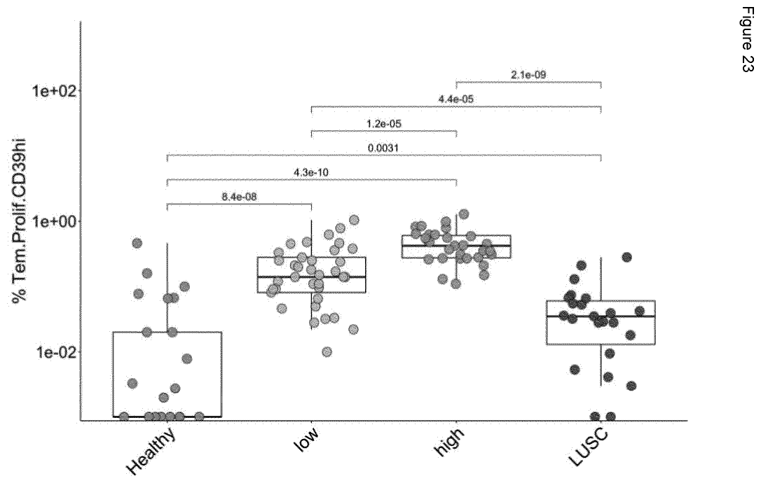

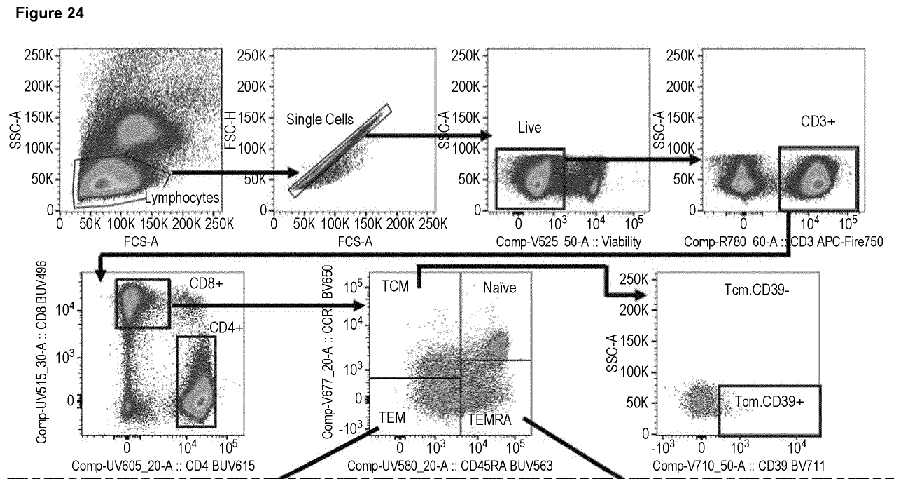

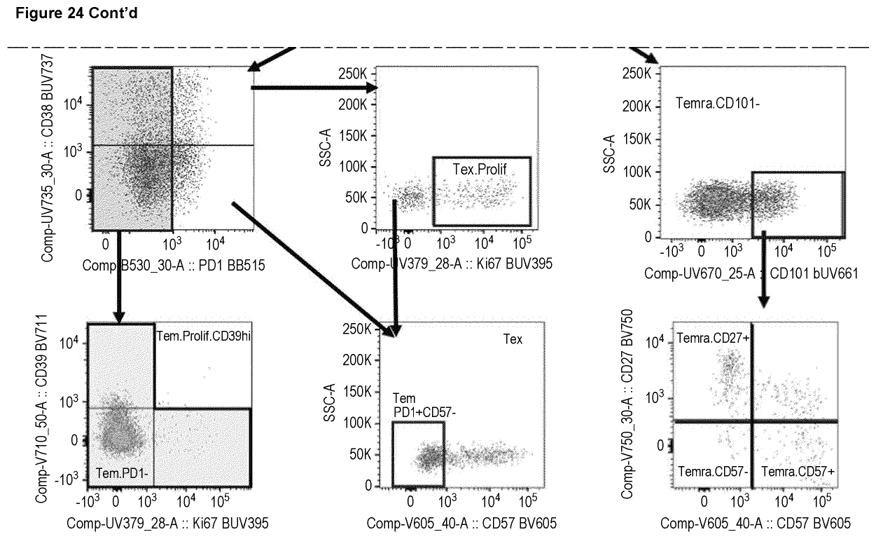

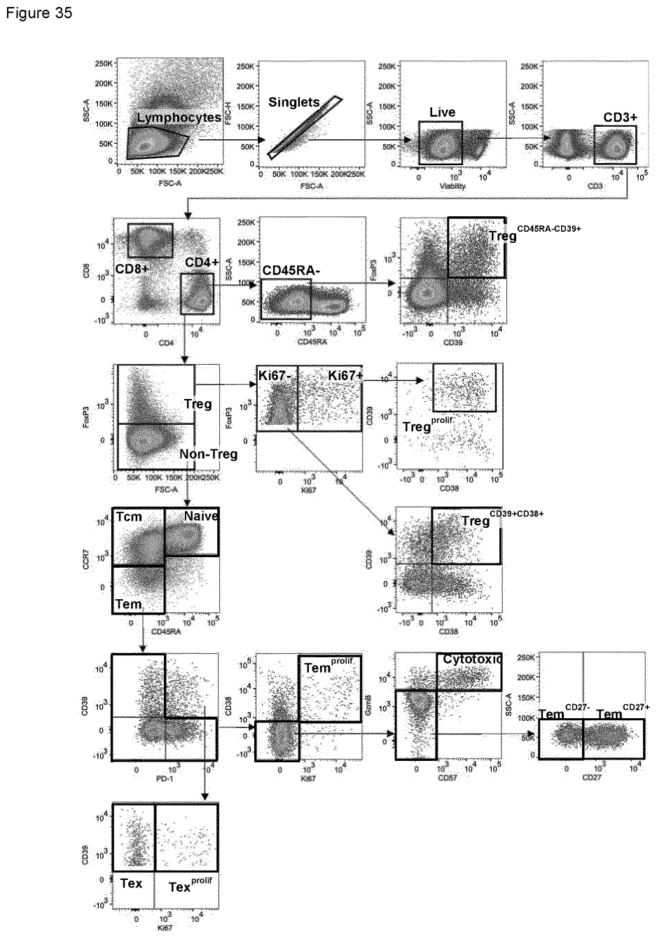

Alternative manual gating methods using a different panel of biomarkers are shown in FIG. 24 and FIG. 35, where CD8 T cells and CD4 T cells (including T regulatory subsets) are analysed separately. These are used for both the pre-invasive and the ASCENT data (see below). In both cases, manual gating frequencies can be used to validate computational clustering frequencies and may be used interchangeably.

### Example 2

The inventors performed further analysis of the data obtained in Example 1 to determine key biomarkers which are to form the inventive panel of biomarkers. A part of this analysis used the biomarkers Ki67 and CD39 to manually gate the clusters of total T cells isolated from a blood sample using CD3. Surprisingly, the expression of these two biomarkers were showed significantly differences between high-grade and low-grade disease. The inventors were able to show that subjects can be separated into high-grade or low-grade disease based on detection of Ki67 and CD39 expression on T cells. FIG. 1. This provides a simple and powerful tool requiring detection of expression of Ki67 and CD39. Based on the expression of Ki67 and CD39 biomarkers, a subject can be identified as being at risk for having a progressing or high-grade pre-invasive lesion or nodule or having a solid tumour. The analysis is based on a subject having more Ki67 and CD39 expressing T cells compared with the T cells in which expression of one or both of Ki67 CD39 is not detected.

### Example 3

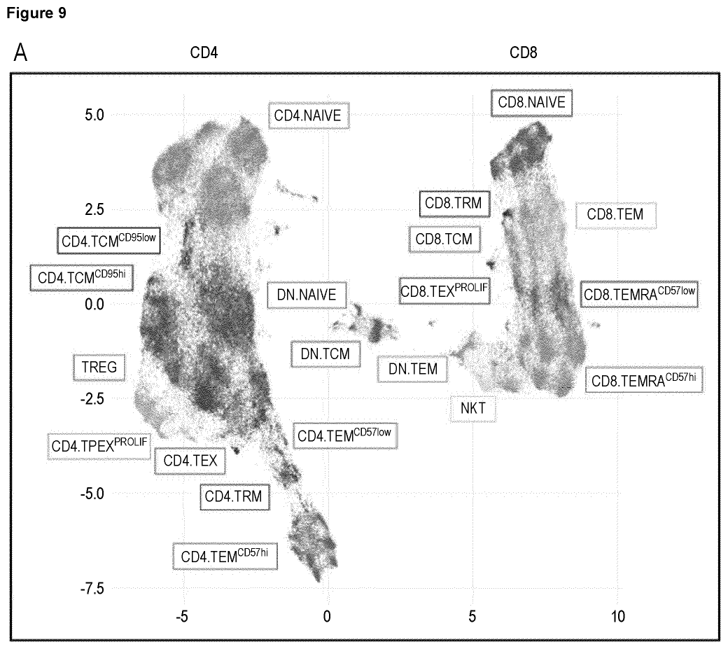

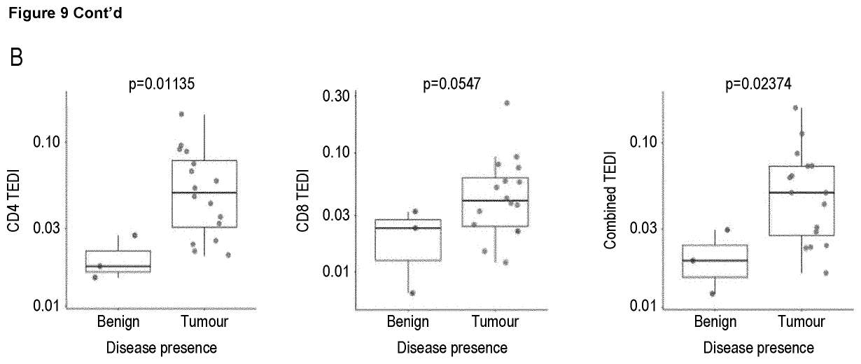

To assess if the methodology demonstrated in Examples 1 and 2 could be used in a cancer type with a different mutational landscape, blinded PBMC samples from patients with benign and malignant renal masses were assessed. T cell subsets were clustered and derived TEDI scores by deriving the ratio of exhausted:resting T cell clusters. FIG. 9. The results show an increase in systemic T cell differentiation in malignant (n=16 samples from 10 patients) vs benign (n=3 samples from 3 patients) disease. A) UMAP of FlowSOM defined T cell clusters from PBMC of all samples stained with 31 biomarkers and analysed by spectral cytometry. 5000 live CD3+ events per samples were down-sampled for analysis. B) The ratio of progenitor vs exhausted CD4 (left), CD8 (centre) or combined (right) T cell subsets. P values from one-tailed, unpaired Wilcoxin test. FIG. 9 shows that the ratio of activated/exhausted:naïve/resting T cells is increased in the blood of patients with renal cancer vs patients with benign disease. These results replicated the flow cytometry findings from pre-LUSC data suggesting broad utility of systemic T cell differentiation in multi-cancer early detection that can be used in all solid tumours from a variety of cancers.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Example 4

Example 4 was performed using TCRseq. The methodology provided below is an example of a method of performing TCRseq. The person skilled in the art will know that other methodologies for performing TCRseq are available, such as is provided in (Rosati E, Dowds C M, Liaskou E, Henriksen E K K, Karlsen T H, Franke A. Overview of methodologies for T-cell receptor repertoire analysis. BMC Biotechnol. 2017 Jul. 10; 17(1):61. doi: 10.1186/s12896-017-0379-9. PMID: 28693542; PMCID: PMC5504616) that may result in different numerical values obtained, but which equate to the same biological results to the Example provided herein. It will be understood to the person skilled in the art that in general, the methodologies and results provided herein in the below Example are not intended to limit the scope of the invention.

### Pre-Processing & Sequencing of the TCR

Blood samples were collected in Vacutainer EDTA blood collection tubes (BD), PBMCs isolated by gradient centrifugation (750 g for 10 minutes) on Ficoll Paque Plus (GE Healthcare). The interface was washed twice with complete RPMI-1640, resuspended in 90% FBS with 10% DMSO (Sigma) and cryopreserved in liquid nitrogen or in a −180 degrees Celsius freezer system prior to staining. To characterise T cell differentiation profiles of HG vs LG samples cryopreserved PBMCs from were thawed with warm R20 media (RPMI with 20% FBS, L-glutamine, HEPES and Penicillin/Streptomycin) and washed with R10 media containing DNase I grade II 37.5 μg/mL (Roche, 10104159001). TCR α-chain and β-chain sequencing was performed by utilizing whole DNA extracted from cryopreserved PBMC samples as above, using the MN NucleoSpin Tissue kit. The samples were sequenced using immunoSEQ from Adaptive Biotechnologies (https://www.immunoseq.com/) (deep sequencing depth). The assay in this Example utilises a multiplex PCR-based methodology that amplifies rearranged TCR CDR3 sequences and exploits the capacity of high throughput sequencing technology, characterises tens of thousands of TCRB CDR3 chains simultaneously.

### Data Processing and Analysis

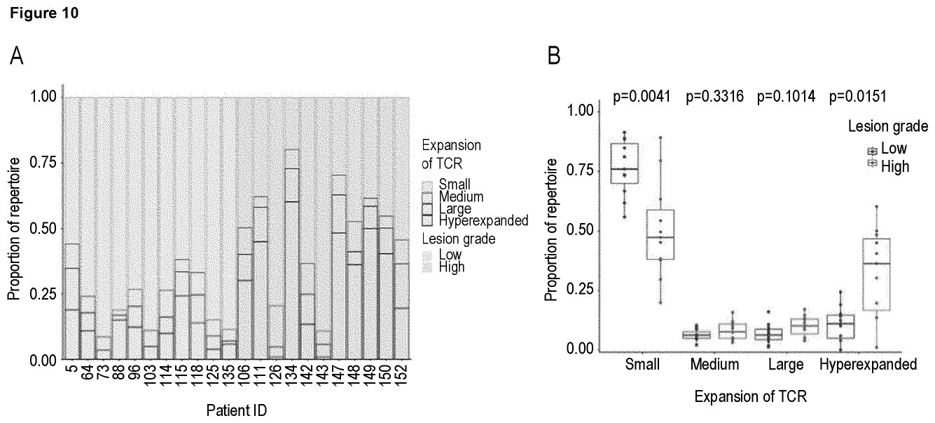

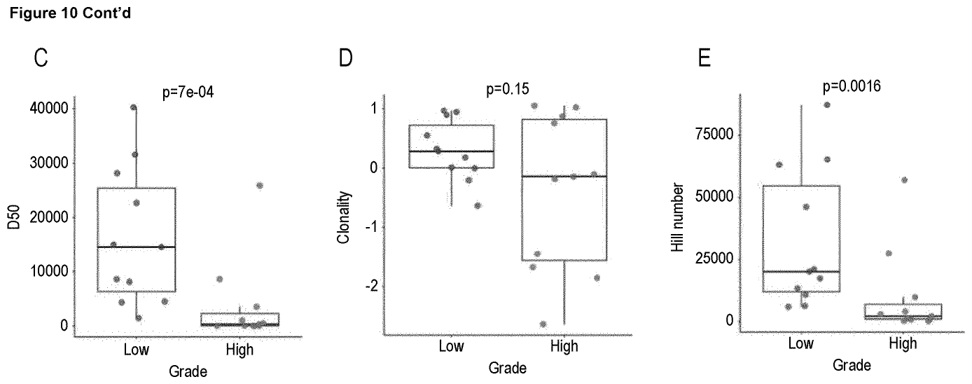

After sequencing, the raw files were passed through our custom analysis pipeline in R, to generate the following metrics: TCR clone expansion, D50 diversity, clonality score, and Hill number (Figure. 10).

### TCR Clone Expansion, D50 Diversity, Hill Number

The raw files were loaded into R and analysed using the Immunarch (v0.6.9) package. TCR clone expansion was calculated using the ‘repClonality’ function with “homeo” specified in the command line. This will provide the proportion of TCRs in the overall repertoire that are defined as hyperexpanded (0.01<X<=1), large (0.001<X<=0.01), medium (1 e−04<X<=0.001), small (1 e−05<X<=1 e−04), or rare (0<X<=1 e−05), where X is a clonotype (FIG. 10A-B). The ‘repDiversity’ function was used to calculate the D50 diversity metric (with “d50” specified in the command line) (FIG. 10C), and the Hill number (FIG. 10E) (again, “hill” specified in the command line). Hill numbers are a mathematically unified family of diversity indices that differ among themselves only by an exponent, q). q=1 was set because it reaches maximal diversity to identify the number of unique TCR CDR3B amino acid sequences. The D50 is a recently developed immune diversity estimate that calculates the minimum number of distinct clonotypes amounting to greater than or equal to 50% of a total of sequencing reads obtained following amplification and sequencing.

### Clonality Score

All TCRseq raw files were imported into R and filtered to only include productive TCR sequences (i.e., no failed sequence reads). Next, the proportion each unique clonotype holds within the repertoire was calculated. After this initial QC step, 1000 TCR sequences were randomly subsampled 1000 times, with the clonality score calculated for each round of sampling, using the entropy (v1.3.1) package. The average clonality score across the 1000 subsamples was taken and transformed into a Z-score, which indicates how much a given value deviates from the mean, within a dataset (FIG. 10D).

### TCRseq Metrics to Predict HG Vs LG Disease

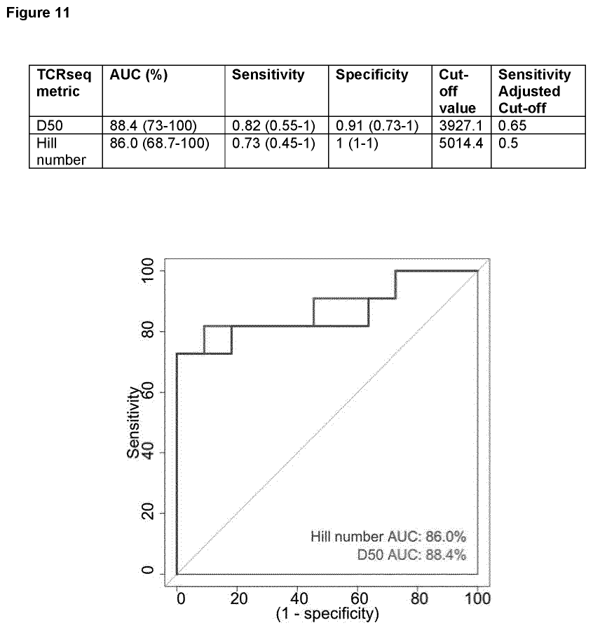

FIG. 10 indicates there is a significant difference in some of these TCRseq metrics between high- and low-grade patients, possibly reflective of persistent TCR engagement from a chronic antigen burden. Receiver operator characteristic (ROC) analysis was completed to test the diagnostic potential of the different metrics using the ‘roc’ function from the pROC (v1.18.0) package (FIG. 11). All possible sensitivity and specificity values with their corresponding threshold probabilities were calculated, as ROC curves represent a trade-off between the true and false positive rates. The threshold probability that yielded the highest combined sensitivity and specificity was selected, known as the Youden Index, a commonly used method to estimate the optimal cut-off point for a diagnostic test. The Youden-determined cut-offs were adjusted to prioritise sensitivity (reducing false negatives) over specificity (increasing false positive), yielding threshold probabilities of the TCR metrics required for correct High grade patient classification. These adjusted cut off values represent the values at which a test using the TCR metrics would be classified as positive or negative. As the D50 had the most significant difference between high- and low-grade disease (FIG. 10C) and has the greatest diagnostic power (FIG. 11), this metric has the highest potential.

Example 4 demonstrates that the state of the T cell repertoire in the blood can be used as a means to determine if an individual is harbouring a progressing or high-grade pre-invasive lesion, nodule or small mass, or having a solid malignant tumour. In this Example, a score beyond a given number indicates a chronic and ongoing immune challenge. It has shown that multiple TCRseq metrics have the potential to be used as a diagnostic predictor. These include:
i) D50 (AUC=88.4%)
ii) Hill number (AUC=86%) (also known as “entropy score”, Shannon index or Shannon entropy score)

In this Example, patients with advanced preinvasive lesions of the lung (likely to progress) vs those with low grade pre-invasive lesions of the lung (unlikely to progress), are more likely to fall below the cut-off values list above for these metrics listed: D50 and Hill number. Whilst utility of these TCRseq metrics is here shown in lung cancer, the underlying principle of blood immune activation linked to an underlying preinvasive nodule/lesion is broad and encompasses all solid cancers, and therefore the utility of this test for early detection is pan-cancer.

### Example 5

The inventors combined T cell flow cytometry data and TCRseq data from the cohort of 30 patients with high or low grade pre-LUSC as described above. Analysis was performed in each platform as described above then combined multiple metrics listed below from each method, using the XGBoost model, via an open-source package in the programming language R. The XGBoost package can be downloaded at https://cran.r-project.org/web/packages/xgboost/index.html

XGBoost is shorthand for Extreme Gradient Boosting, which is a scalable, distributed gradient-boosted decision tree (GBDT) machine learning library. XGBoost provides parallel tree boosting and is the leading machine learning library for regression, classification, and ranking problems.

### ML/AI Analysis Workflow

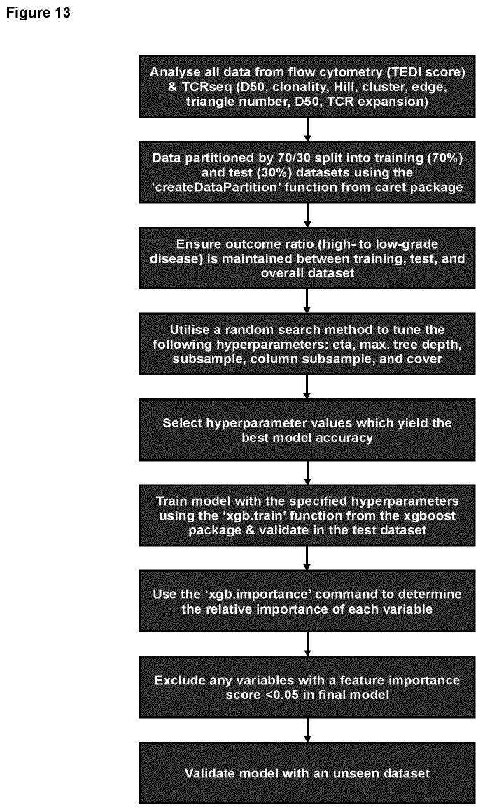

Combining data involves adding values from each set of analysis into a machine learning library. 70% of the data was used to train the model, wherein the library computes which combination of metrics from each analysis best classifies group 1 vs group 2 via an iterative process using decision trees, in which the best decision is boosted until the model is optimised. These 2 groups could be patients with vs without cancer or in the present examples patients with low vs high grade preinvasive neoplasia. The trained model is then applied to the remaining (‘unseen’) 30% of the data to test that it works on a different set of samples. The steps are outlined in FIG. 13.

### Input Data

All metrics were added from the flow cytometry (e.g., as described in Examples 1 and 2) and TCRseq analysis (e.g., from Example 4) to train the machine learning model. TCRseq variables included: D50 diversity metric, Hill, and TCR expansion metrics (i.e., small (1 e−05<X<=1 e−04), medium (1 e−04<X<=0.001), large (0.001<X<=0.01), hyperexpanded (0.01<X<=1) expansion proportions). CD4 and CD8 TEDI scores were derived from the flow cytometry metrics as described above.

### Data Processing

All analysis was completed in R (v1.4.1106). The data was partitioned into a training and a test data set with a 70/30 split, using the ‘createDataPartition’ function from the caret package (v6.0-92). The inventors ensured the outcome ratio (i.e., the high to low grade ratio) was maintained in both data sets. Outcome was defined as a high/low grade (1/0) lesion for each patient.

### Hyperparameter Tuning

To select the optimum values for the hyperparameters to yield best model accuracy, a random search method was completed, where 10000 iterations of the model are generated with different values for each of the defined hyperparameters (maximum tree depth: 2-6; eta: 0.01-0.3; subsample: 0.7-1; subsample ratio of columns: 0.6-1; cover: 0-10). We selected the hyperparameter values which yielded the greatest model accuracy for the final, tuned model. The final model was trained using the ‘xgb.train’ function from the xgboost (v1.6.0.1) package.

### Feature Importance

The ‘xgb.importance’ function was used to determine the relative importance of each variable within the overall model. Any variables with a feature importance score <0.05 were excluded.

### Results

FIG. 3 shows that the flow cytometry defined CD4 TEDI (AUC=94.2%), CD8 TEDI (AUC=92.6%) are strong classifiers of patients with high vs low grade pre-LUSC. FIG. 10 also shows that the TCRseq defined measures, including the D50 index (AUC=88.4%) have discriminatory power to specifically detect patients with high grade pre-LUSC. The drawing then shows that combining flow cytometry and TCRseq metrics in the XGBoost package yields improved classification of high-grade vs low-grade disease (AUC=100%). This demonstrates that combining multiple different metrics from T cell analyses in the blood can generate a powerful novel tool that detects preinvasive lesions which are likely to progress to lung cancer. In principal this could be applied to other solid cancer types.

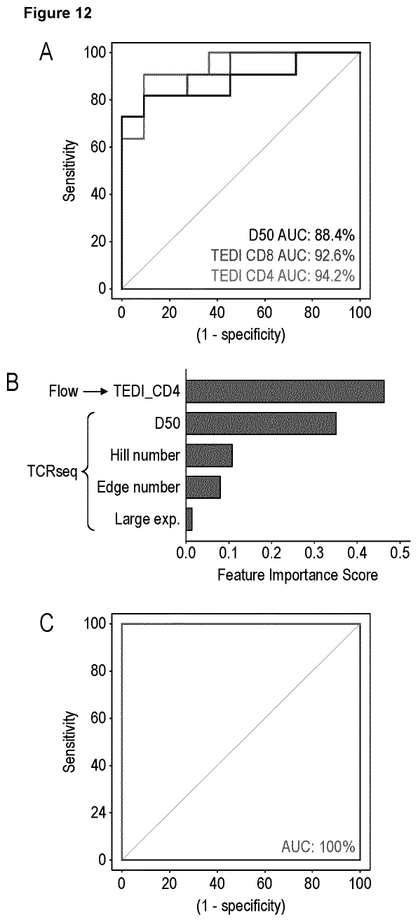

In more detail the figures show that there are multiple CD4 and CD8 T cell phenotypes in the blood of patients with preinvasive lung disease, FIG. 3A. Patients with high grade disease have more activated and exhausted CD8 and CD4 T cells, and fewer naïve and resting CD4 and CD8 T cells, FIG. 3C. This can be converted into ratio(s) which divides these patient populations, FIG. 3D. In addition, TCRseq analysis shows that patients with high grade disease have larger clonal T cell expansions and fewer small T cell expansions and lower TCR diversity, FIGS. 10A, 10B and 10C. Finally, that these can be combined in the XGBoost model with superior performance to metrics from either assay alone, where the top ranking features of each assay are shown to be the CD4 TEDI (Flow cytometry) and D50 index (TCRseq), FIGS. 12A, 12B and 12C.

FIG. 12A demonstrates that both cytometry and TCRseq approaches can be used to effectively distinguish subjects into high-grade and low-grade disease. These techniques can also be combined together, incorporating multiple metrics from these techniques into a single analysis, to generate data that further improves distinguishing subjects into high-grade and low-grade disease, as demonstrated with ROC analysis FIG. 12B-C. This is demonstrated by the AUC increasing from 88% with TCRseq analysis alone to 100% with TCRseq analysis+flow cytometry analysis, see FIG. 12. AUC of flow cytometry analysis alone is 93-94% but adding TCRseq in a combined model shows 100%. This is the comparison between FIG. 12a and FIG. 12c. Therefore FIG. 12 shows for the first time that combining different measures of blood T cell differentiation results in a stronger blood test than using each alone. This would lead to an improved method of detecting cancer or nodules or lesions or masses that lead to cancer compared to a test that used either technology alone.

### Further Exemplification—Detecting Biomarkers Expression of T-Cells in the Blood Using Flow Cytometry

### Ascent

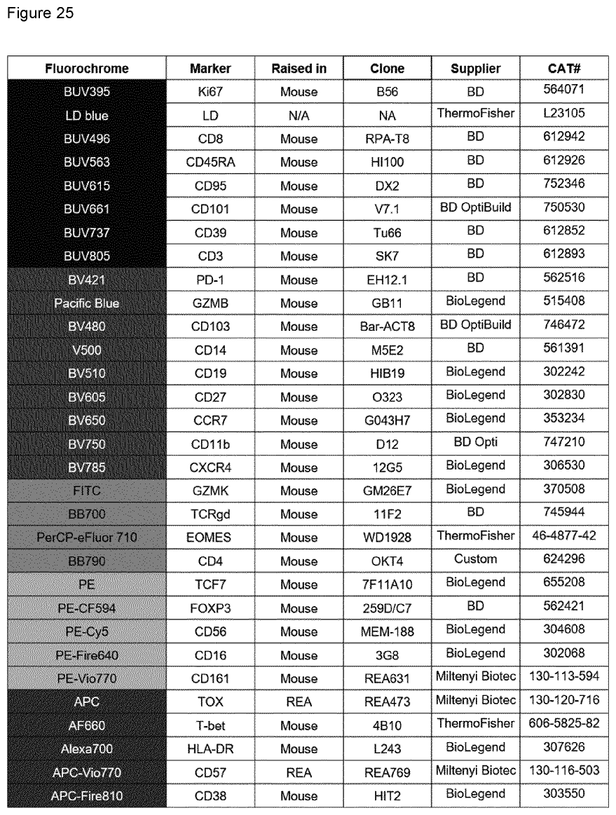

ASCENT is an observational study of patients with screen-detected lung cancer through the SUMMIT longitudinal surveillance study via LDCT scans. Blood (and tissue) samples are collected from surgery at the point of resection. For the ASCENT flow analysis, 86 patients were included, of which there were 57 NSCLC (35 LUAD (Lung adenocarcinoma)+22 LUSC (Lung squamous cell carcinoma) and 29 Healthy. Healthy age-matched blood samples were collected from patients going in for orthopaedic surgery, with no active infections or auto-immune conditions. ˜90% of NSCLC samples used in ASCENT analysis are Stage I (51 Stage I, 4 Stage II, 2 Stage III). The flow cytometry panel used for this data-set is shown in FIG. 25. Samples were acquired on a ID7000 Spectral Cell Analyser from Sony Biotechnology.

### Example 7

The findings relating to skewing of T cell differentiation in the blood from pre-invasive patients were validated in a larger cohort of patients with early-stage (mainly stage I), established non-small cell lung cancer (NSCLC).

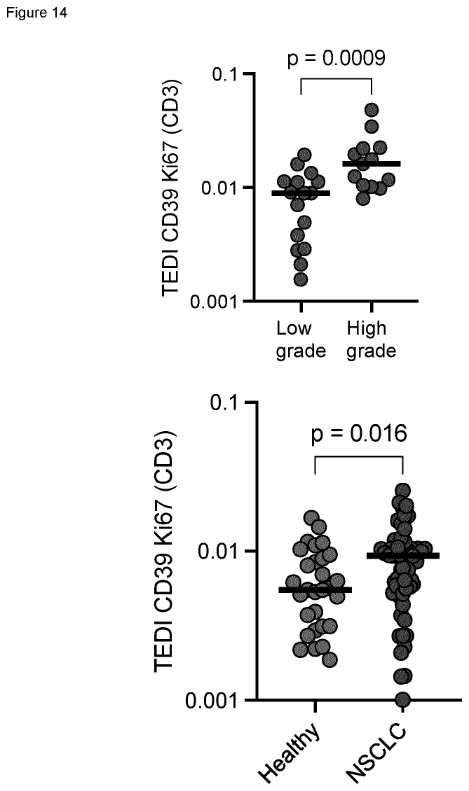

As shown by the bottom of FIG. 14, in early stage established disease, a higher ratio of activated:resting T cells is also observed as we did in pre-invasive disease. This is demonstrated by the TEDI CD39 Ki67 (CD3), where NSCLC patients have a higher proportion of CD3 CD39+Ki67+ cells than healthy. A similar plot with healthy patients versus LUSC gave a p-value of 0.14.

As shown in FIGS. 15A and 15B, when combining pre-invasive and NSCLC data, we also see an increase in the TEDI CD39 Ki67 ratio from healthy to low-grade to high-grade. The signal peaks at high-grade and dropping off at the lung cancer stage. This suggests the high-grade pre-invasive stage has the potential to be the most immunogenic and is therefore the optimum time to detect malignancy in the blood.

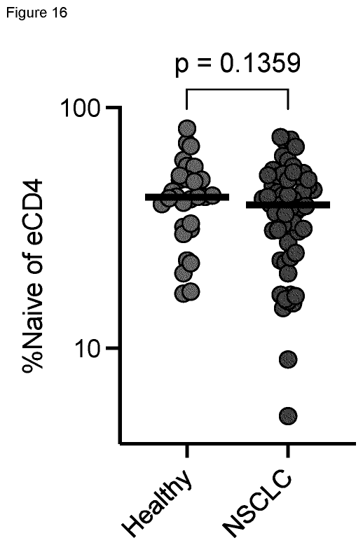

As well as lower frequency of the biomarkers CD39 and Ki67 in T cells in lung cancer patients, we also observe a trend of lower frequencies of naïve CD4+ T cells in NSCLC patients compared to healthy (p=0.1), similar to what was previously observed in the pre-invasive setting (see FIG. 16). When combining healthy and low grade (n=66) and combining high grade and HSCLC (n=88), there is still a significantly higher CD3 TEDI score in the disease/high-risk group compared with the healthy/low-risk group (see FIG. 17) coupled with a decrease in naïve cells in the CD4 T cell compartment.

### Example 8

In further analysis of the pre-invasive flow cytometry data, the present inventors noted the importance of CD4+ regulatory T cells (Tregs) expressing the biomarker FoxP3. Proliferating Tregs may also express the activation/proliferation markers CD39 and Ki67. In both pre-invasive and early-stage lung cancer, there was a significant increase in CD39+Ki67+ Tregs in high-grade/early-stage lung cancer when compared with low-grade/healthy respectively. This is in accordance with more activated/memory cells supporting the finding that T cell differentiation is skewed during pre-invasive and invasive lung cancer, wherein patients having a higher risk of having a progressing/high-grade lesion/nodule/tumour to higher have frequencies of more activated/effort/regulatory T cells. This data is shown in FIG. 18. When combining this data together, there is an increase from healthy to low grade and to high grade, with the signal from activated/proliferating Tregs peaking in high grade pre-invasive samples (see FIG. 19). When combining healthy and low grade (n=66) and combining high grade and HSCLC (n=88), there is still a significantly higher frequency of total Tregs, CD39+Tregs and CD39+Ki67+ T regs (see FIG. 17).

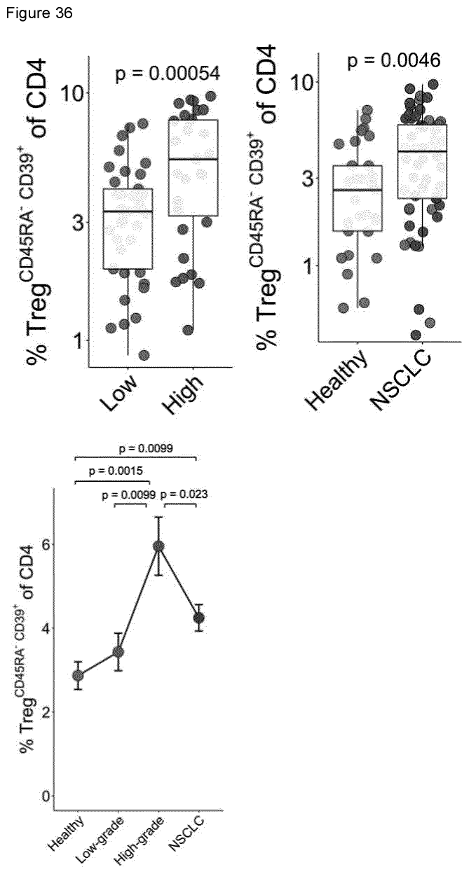

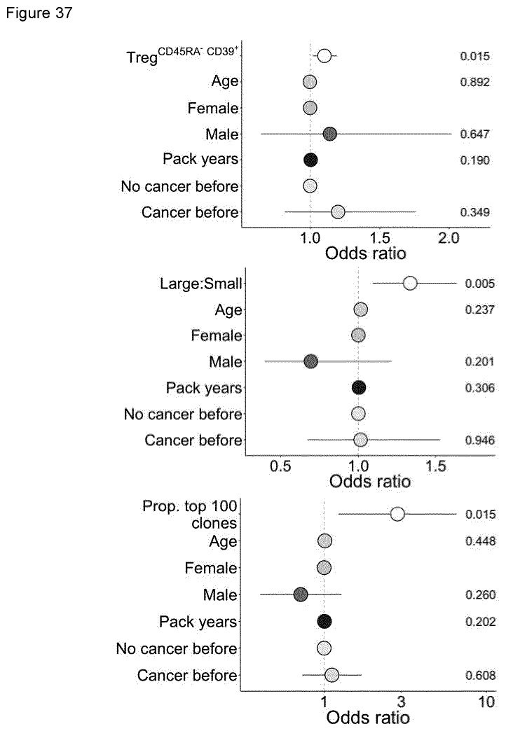

Tregs (i.e., CD4+ T cells expressing the biomarker FoxP3), further expressing the biomarker CD39 (CD39+) in the absence of the biomarker CD45RA (CD45RA−) were also significantly enriched in CD4+ cells in high-grade/early-stage lung cancer when compared with low-grade/healthy respectively (see FIG. 36), also peaking in high pre-invasive samples.

### Example 9

Another subpopulation of T cell, i.e., Tem.Prolif.CD39hi cells, where Prolif=Ki67+, expressing both CD39+ and Ki67+ were also significantly enriched in high-grade samples in live CD8 Cells (CD3+CD8+ cells). These cells contain the presence of CD39 and Ki67 biomarkers, as well as having negative expression of the biomarkers CD45RA, CCR7 and PD1.

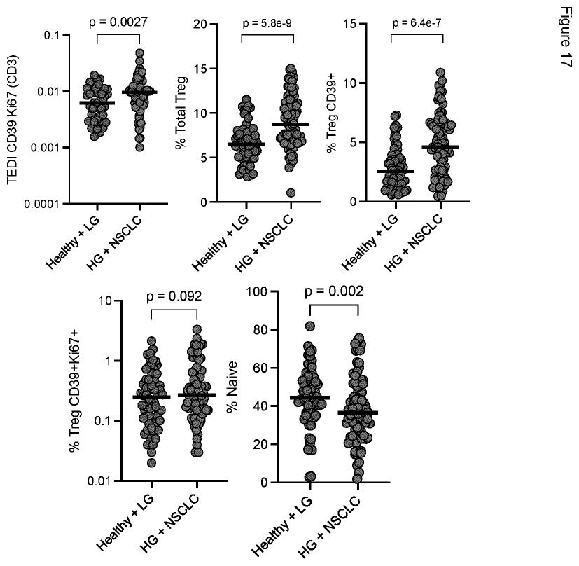

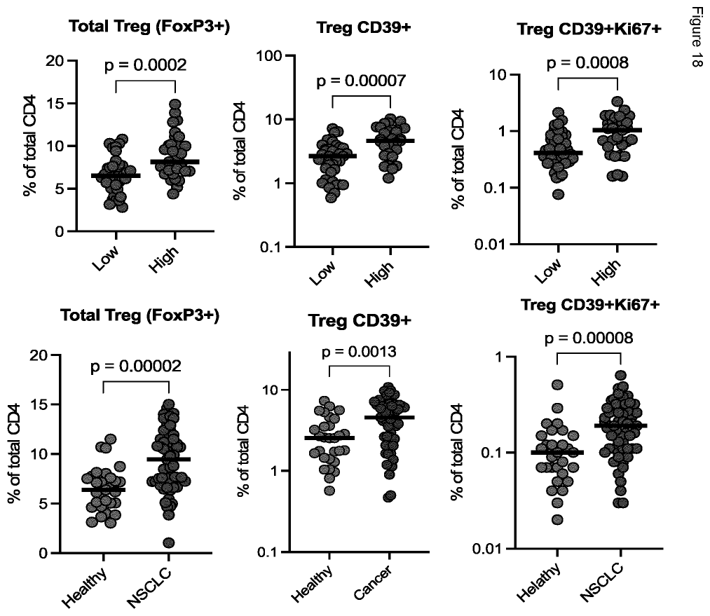

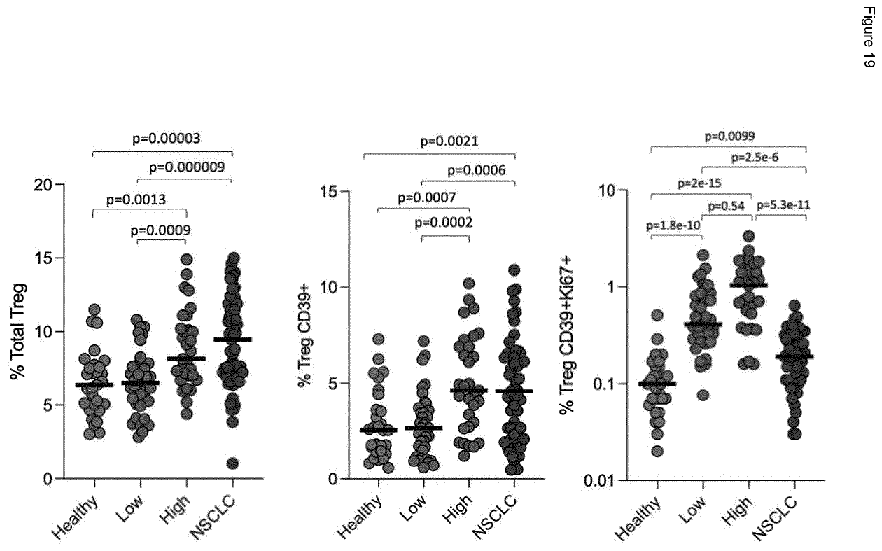

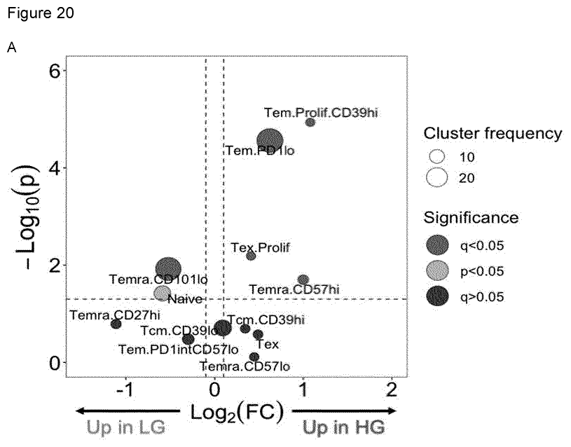

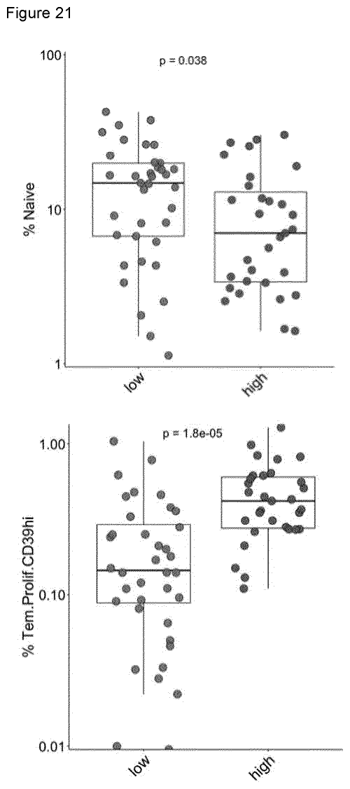

For the pre-invasive (PID) data, flow cytometry analysis is of 68 samples (31 high grade, 37 low grade) which passed the QC for having >3000 Live CD3+ and CD8+ cells used for analysis. FIG. 20 shows a volcano plot of populations determined by manual gating on flow cytometry that are significantly enriched in high grade pre-invasive lesions or nodules or low grade pre-invasive lesions or nodules. Significant enrichment of the Tem.Prolif.CD39hi cells (prolif=Ki67+) was observed in high grade pre-invasive lesions or nodules. FIG. 21 shows box plots for Top) naïve T cells and Bottom) the above-mentioned Tem.Prolif.CD39hi populations (right) at sample level.

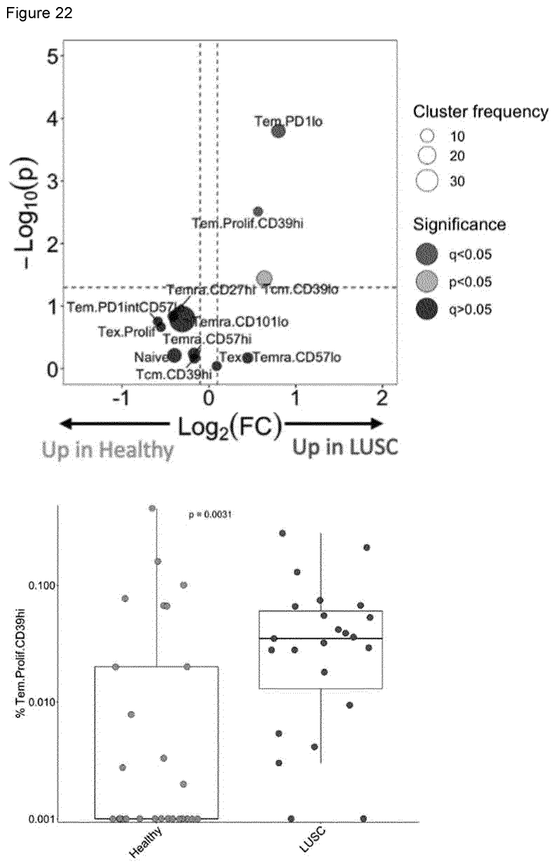

For the ASCENT data, the flow cytometry analysis is of 49 samples (27 Healthy and 22 LUSC) which passed the QC for having >1500 Live CD3+CD8+ cells were used for analysis. FIG. 22 (Top) shows a volcano plot of populations determined by manual gating that are significantly enriched in LUSC. FIG. 22 (Bottom) shows a box plot for the Tem.Prolif.CD39hi population which is significantly enriched in LUSC (Lung squamous cell carcinoma).

FIG. 23 shows the combined box plot values from both the PID (pre-invasive data) and ASCENT flow cytometry analysis to display the change in Tem.Prolif.CD39hi population.

FIG. 24 shows the flow cytometry gating strategy for both PID (pre-invasive data) and ASCENT manual gating used in this particular Example.

### Further Exemplification—Determining the Diversity (i.e., Using Diversity and Clonality Metrics) of T-Cells in the Blood Using TCR-Seq

### Example 10

The findings relating to the remodelling of the T cell receptor repertoire in the blood from pre-invasive patients were validated in a larger cohort of patients with early-stage, established non-small cell lung cancer (NSCLC) to discern patients with progressive pre-invasive disease or early-stage invasive neoplasia (Stage 1 NSCLC).

As described above, ASCENT is an observational study of patients with screen-detected lung cancer through the SUMMIT longitudinal surveillance study via LDCT scans. Blood (and tissue) samples are collected from surgery at the point of resection.

For ASCENT TCR-seq analysis, 75 patients were included: 57 NSCLC (35 LUAD+22 LUSC), and 18 healthy subjects. Healthy age-matched blood samples were collected from patients undergoing orthopaedic surgery, with exclusions to ensure no active infections or auto-immune conditions. ˜90% of NSCLC samples used in ASCENT analysis are rarely sampled very early Stage I tumours (51 Stage I, 4 Stage II, 2 Stage III).

This validation cohort was analysed as before. For TCRseq DNA was extracted from peripheral blood samples and sequenced using the same platform (Adaptive ImmunoSeq), and pre-processed and analysed using Immunarch package on R.

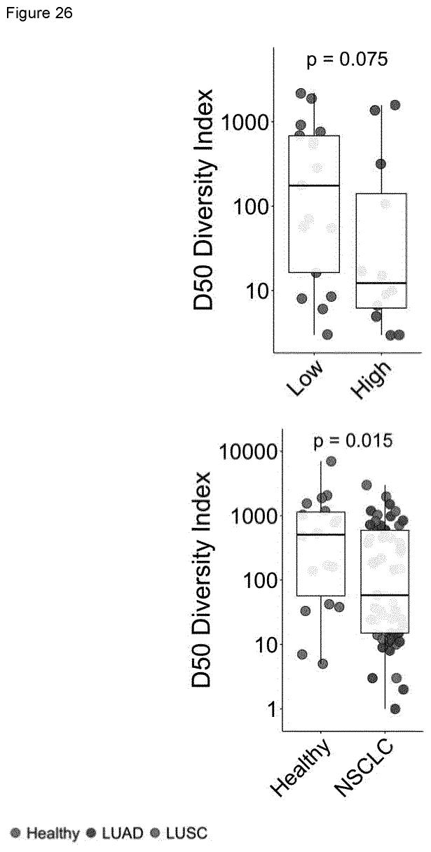

In further analysis of pre-invasive data, a trend in D50 diversity index (higher score in low-grade samples) and clonality (higher score in high-grade samples) was observed. Looking at the same metrics in ASCENT (established very early-stage lung cancer), the same trends were observed when comparing healthy to NSCLC (see FIG. 26)

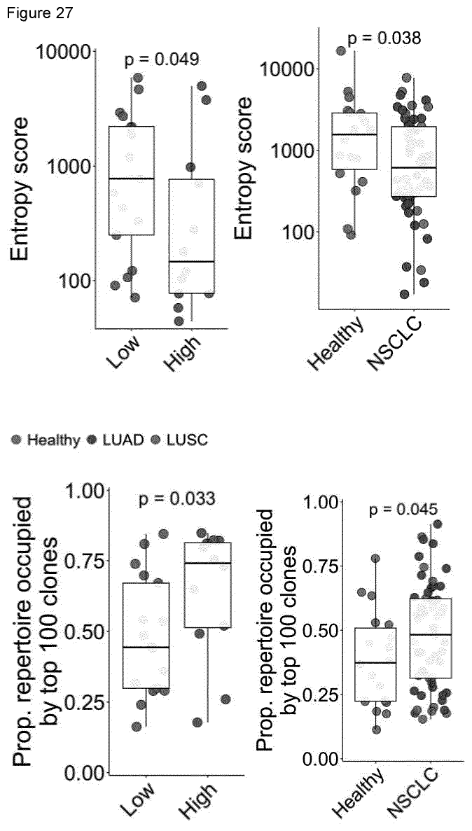

Other metrics measuring diversity and clonality of the repertoires also showed the same trends as the pre-invasive analysis including D50 and the proportion of the repertoire occupied by the top 100 clones as a proxy for clonality, with even stronger results. The Hill score (aka entropy score) and the proportion of the repertoire occupied by the top 100 clones further show the same differences between low-vs high-grade, and healthy vs NSCLC (see FIG. 27).

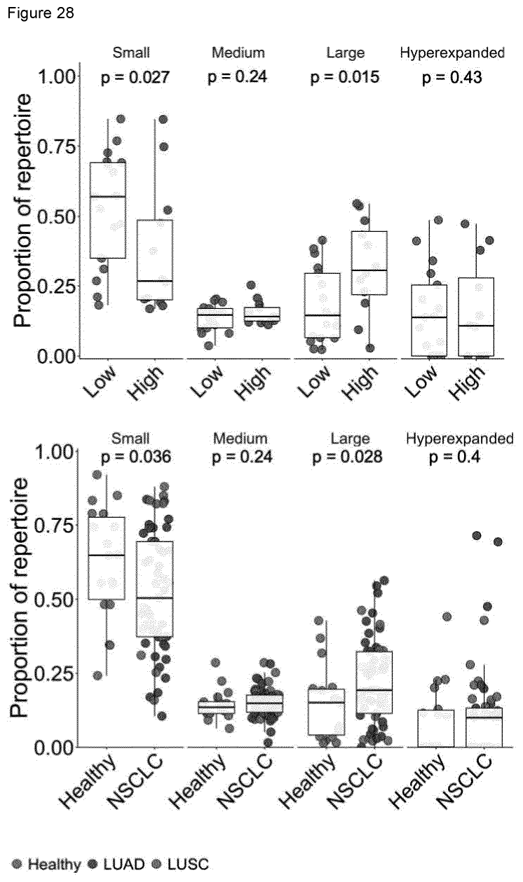

Looking at the proportion of the repertoire occupied by small (<0.01% of repertoire), medium (0.01-0.1% of repertoire), large (0.1-1% of repertoire) or hyperexpanded (>1% of repertoire) clones, an increase in large clones was observed present in the blood of high-grade/progressive pre-invasive patients and established early-stage invasive neoplasia, coupled with a decrease in small clones in low-grade/non-progressive pre-invasive patients and healthy volunteer samples. This demonstrated that an increased proportion of large clones occupying the subject's peripheral TCR repertoire can also be used as an indicator of higher risk of progressing into lung cancer, and/or the presence of a progressive lesion/nodule or early-stage tumour (see FIG. 28)

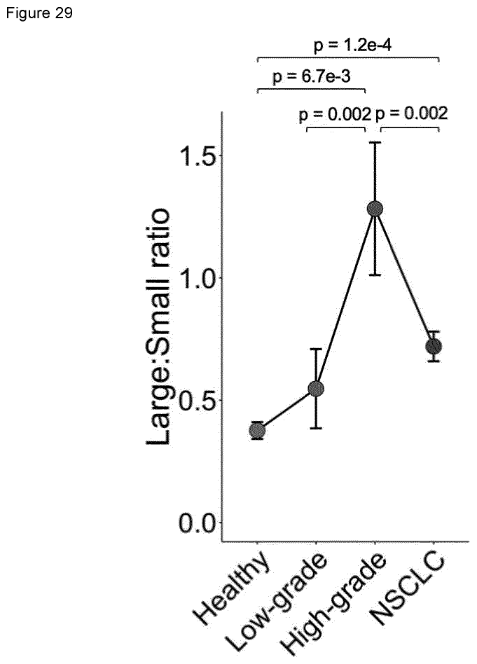

Similar to the phenotypic “TEDI” ratio used in the flow cytometry data representing a shift in differentiation skewing in the blood, a ratio of large:small clones occupying the repertoire can be used to distinguish healthy vs pre-invasive disease vs early-stage NSCLC, with a new metric of clonal expansion. This signal can be tracked as carcinogenesis develops, with the ratio signal peaking at high-grade/progressive lesions. This demonstrates the technique's potential to be used in early detection of cancer (i.e., before cancer formally develops) (see FIG. 29).

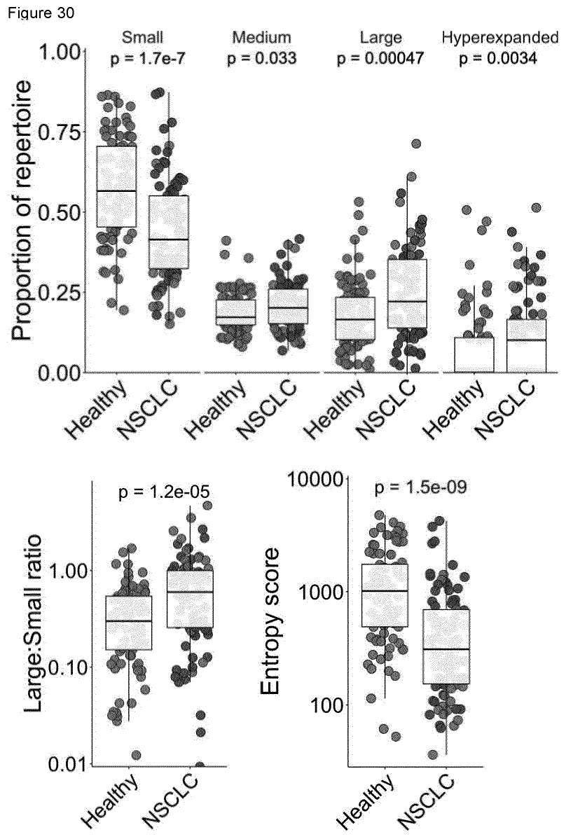

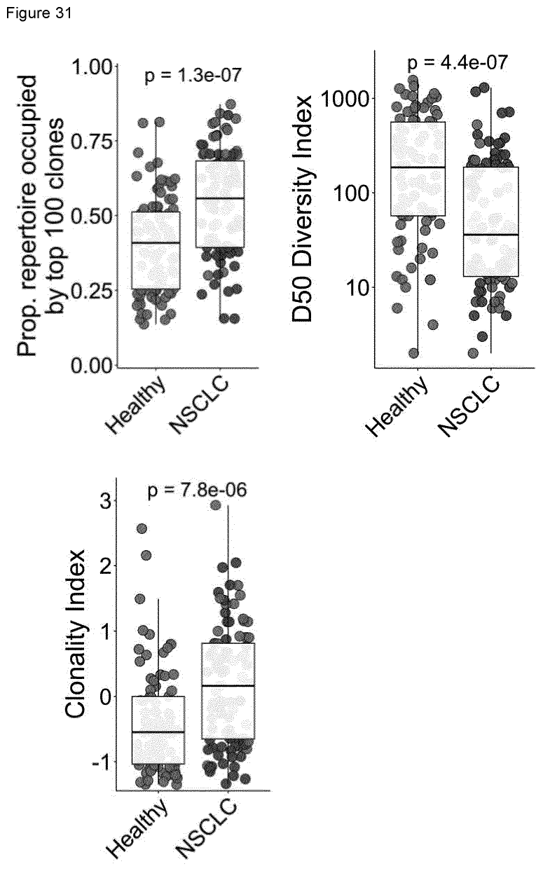

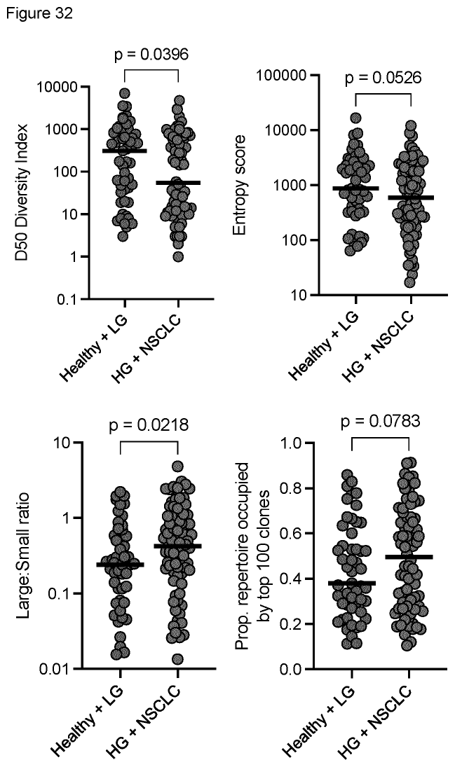

The same trends were also observed when this novel analysis was performed on a publicly available large scale data set acquired via the same wet lab and bioinformatic platform comparing healthy subjects and NSCLC patients (see FIGS. 30 and 31). Combining healthy+low-grade (no tumour and low risk of developing a tumour), and high-grade+NSCLC (high risk or with early stage cancer) therefore allows for a comparison of no/low risk of developing or currently having lung cancer to high risk of developing or currently having lung cancer in situ. This comparison aids the stratification of the general population using peripheral blood for bulk TCR-seq. The trends in the output metrics described above in each cohort remain the same when combined. Generally, the repertoires of higher risk (HG+NSCLC) patients are more clonal and expanded (higher clonality index, higher proportion of the repertoire occupied by top 100 clones, higher large:small expansion ratio), and less diverse (lower D50 index, lower entropy score/hill number) than the repertoires of lower risk (Healthy+LG) patients. Notably, the large:small expansion ratio (which calculates a ratio similar to the phenotypic ratio of exhausted/activated T cells to naïve/resting cells) was found to be the most significant metric to distinguish these two groups (see FIG. 32).

### Further Exemplification—Combining Flow Cytometry and TCR-Seq in a Multi-Omic Approach.

The following findings provide further evidence of using a multi-omic model using the combination of the updated T cell phenotype and TCR-seq signals in the blood for early detection of cancer.

### Example 11

Using follow-up time available from the pre-invasive cohort, we used a combination of the flow cytometry phenotypic signal which uses some of our key biomarkers to differentiate between sample types (e.g., FoxP3, CD39+ to identify effector Treg cells) and the ratio of large:small clones from the TCR-seq analysis to predict the probability of a patient receiving a lung cancer diagnosis. A patient's effector Treg CD39+ frequency is multiplied by their large:small TCR ratio and all patients (low- and high-grade) are split by their median multiplied score.

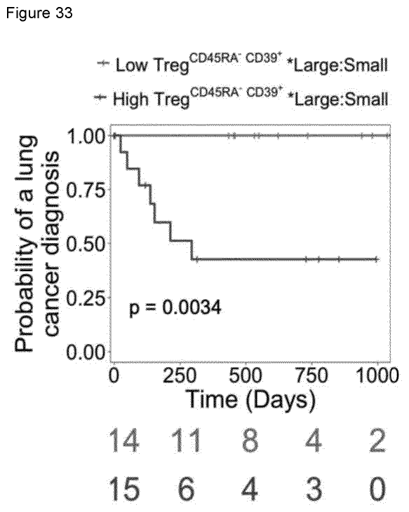

Kaplan-Meier analysis of this combined metric from flow cytometry and TCR-seq shows a significant difference when future lung cancer diagnosis is used as outcome, demonstrating the predictive/diagnostic potential when these two techniques (i.e., TCR-seq and flow cytometry) are used in combination. It is also important to note that using both metrics in combination give stronger predictive potential than using either the flow cytometry data or TCR metrics alone. After accounting for clinical data, this combined flow/TCR-seq metric is able to predict a future lung cancer diagnosis, further highlighting the diagnostic potential of the mentioned peripheral immune metrics (see FIG. 33).

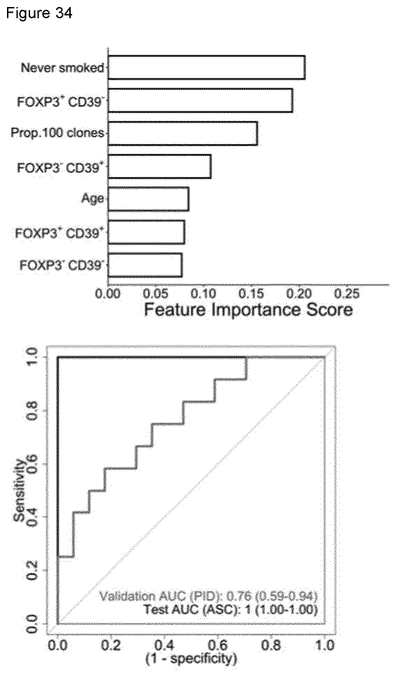

We updated the prior-mentioned machine learning model XGBoost, including the different TCR-seq metrics (proportion of repertoire occupied by top 100 clones, hill number, small clones, hyperexpanded clones), flow cytometry metrics (FoxP3 vs CD39 manually gated quadrant frequencies on total CD4, i.e., FoxP3-CD39−, FoxP3-CD39+, FoxP3+CD39−, FoxP3+CD39+), and standard clinical metrics (age, gender, smoking status). The model was now trained on 70% of the ASCENT cohort, tested in the remaining 30% of ASCENT, and then validated on 31 patients from PID. (Previously the model was trained and tested on 22 PID patients, but with no validation cohort). Test AUC in ASCENT was 100%, and validation AUC in PID was 76%. As demonstrated by ROC analysis, the combination of these techniques alongside standard clinical metrics into a single analysis show great promise in distinguishing low- from high-grade subjects. The relative importance of each metric used (as calculated by the feature importance score) shows that peripheral immune metrics that contribute more towards the model than clinical variables such as age, which is already commonly and strongly associated with lung cancer development (see FIG. 34).

### Materials and Methods

### High-Dimensional Clustering

Clustering of flow cytometry data was completed following a modified pipeline from Nowicka et al [https://f1000research.com/articles/6-748]. FCS files underwent quality control of signal acquisition, assessed by the FlowAI package (v1.24) [https://academic.oup.com/bioinformatics/article/32/16/2473/2240408?login=false]. The arcsinh transformation was applied to the data, using the prepData function from the CATALYST package (v1.18.1) [https://www.ncbi.nlm.nih.gov/pmc/articles/PMC5981006/], with the cofactor set to 150 for standard fluorescence flow cytometry (PID cohort) and to 550 for spectral flow cytometry (ASCENT cohort). Any markers with a poor contribution to phenotypic variance were excluded pre-clustering. These were determined using the PCA-based non-redundancy score (NRS) as described in the Nowicka et al [https://f1000research.com/articles/6-748]. pipeline. CD103, Tim-3, TCF-7, CXCR4, TIGIT, and CD4 were excluded from clustering due to poor marker separation in the PID cohort, as well as the ASCENT cohort for consistency between panels (CD4 excluded as FCS files only included CD4+ T cells).

Data was clustered using the FlowSOM package (v2.2.0) [https://onlinelibrary.wiley.com/doi/full/10.1002/cyto.a.22625] onto a 10×10 node square self-organising map (SOM). Nodes were clustered by the ConsensusCenterPlus package (v1.58.0) [https://www.ncbi.nlm.nih.gov/pmc/articles/PMC2881355/], as described by Nowicka et al [https://f1000research.com/articles/6-748]. The UMAP algorithm was applied as a dimension reduction analysis to characterise the phenotypic relevance between individual clusters. The resulting clusters were manually merged based on marker expression similarity, occupation of space on the UMAP, and previously defined T cell states.

### Manual Gating

Subsets resolved from high-dimensional clustering were manually identified by conventional biaxial gating to ensure validity of clusters. PBMCs with ≥x viable T cells were analysed (PID cohort ≥5000 CD4; ASCENT cohort ≥4000 CD4). Manual gating analysis was carried out on FlowJo v10.8.1. Populations gated are shown on a concatenated file of all samples from one batch. Frequencies from total CD4 can be used to validate cluster significance from the high-dimensional clustering pipeline analysis

### TCR Sequencing

Sequencing of the CDR3 regions of PBMC TCR-β chains was performed using the immunoSEQ® Assay (Adaptive Biotechnologies, Seattle, WA) as previously described [https://www.ncbi.nlm.nih.gov/pmc/articles/PMC7418738/, https://www.nature.com/articles/s41467-019-14273-0 #Sec12]. In brief, extracted genomic DNA was amplified in a multiplex PCR, followed by high-throughput sequencing. Raw files were processed using the MixCR pipeline [https://www.nature.com/articles/nmeth.3364] and exported as tsv files.

Curation of publicly available TCRseq dataset Publicly available data from the immuneACCESS portal (Adaptive Biotechnologies, Seattle, WA) was leveraged to curate an additional validation dataset that had been sequenced on the sample platform as the PID and ASCENT samples. Healthy controls were obtained from the immunoSEQ hsTCRB-V4b Control Data [https://doi.org/10.21417/ADPT2020V4CD]. The non-small cell lung cancer cohort was taken from Reuben et al. [https://www.nature.com/articles/s41467-019-14273-0 #Sec2].

### Analysis of TCR Sequencing Data

Raw tsv files were processed to include only productive sequences. To pass QC, repertoires required a minimum of 1000 unique clones and <3 counts per CDR3 sequence to limit the effect of naïve cells and sequencing artefacts.

The ‘repClonality’ function was used to calculate clonal expansion metrics from the immunarch package (v0.6.9) [https://zenodo.org/record/3367201]. To calculate the Large:Small ratio, the proportion of clones deemed as “large” (1%<repertoire >0.1%) was divided by the proportion of clones deemed as “small” (<0.001%). The diversity metrics D50 and Entropy/Hill score were calculated using the ‘repDiversity’ function from the same package, with the method set to “d50” and “hill”, respectively. For the Entropy score, Q=1 was selected.

The Renyi entropy is a generalised measure of diversity as previously described [Joshi paper]. Briefly, as the Renyi diversity approaches 0, greater importance is given to smaller/rarer clones but, as Renyi diversity tends to infinity, greater importance is given to the more common clones. The Renyi diversity was calculated as a range of values using the ‘renyi’ function from the vegan (v2.6-4) [https://CRAN.R-project.org/package=vegan] package. All repertoires were randomly sampled to 1000 unique sequences, 100 times before the mean Renyi score was calculated for each sequence for each value of Renyi diversity.

To calculate the clonality index, all productive sequences for each repertoire were down-sampled to 1000 clones. The clonality was estimated for this sample using (1-[‘entropy’ function]) from the entropy package (v1.3.1) [https://CRAN.R-project.org/package=entropy]. This was bootstrapped 1000 times and the mean clonality score was taken for each sample.

For TCR clustering, the top 1000 clones from each patient from all 3 cohorts were downsampled and subsequently clustered together using the ‘gliph2’ function from the turbogliph package (v0.99.2) [https://www.nature.com/articles/s41587-020-0505-4]. Clusters were assigned to a histology group based on a count proportion threshold of >50%. Using publicly available databases like VDJdb (URL), viral cluster specificity was assigned. Only one sequence within a cluster required a specificity annotation for the whole cluster to be deemed as viral.

### Statistical Analysis

All statistical tests were performed in R (version 1.4.1106). Tests involving differences between groups were done using a ‘wilcox.test’ using unpaired filters, or a linear mixed effects model from the Ime4 (v1.1-30) [https://www.jstatsoft.org/article/view/v067i01] package. Details of the statistical test used are typically outlined in the corresponding figure legends. When required, p-values were adjusted using Benjamini-Hochberg (BH) p-value correction [doi: 10.1214/193940307000000158], using the ‘p.adjust’ function. Correlation analyses completed using the ‘cor.test’ function, with the method set to ‘Spearman’ and the alternative ‘two.sided’. Logistic regression models to account for potentially confounding variables were completed using the ‘glm’ function. Coefficients were exponentiated to get odds ratios with 95% confidence intervals. Significance was determined with a Wald's test, calculated by taking the cumulative probability associated with the absolute value of the Wald test statistic. All Wald's test p-values are two-tailed. All graphical presentation completed using the ggplot2 package (v3.3.6) [H. Wickham. ggplot2:Elegant Graphics for Data Analysis. Springer-Verlag New York, 2016.].

### Future Lung Cancer Diagnosis Analysis

Patients were split into groups (low/high) based on the median metric value. Kaplan-Meier (KM) analysis was completed using the ‘survfit’ function from the survival package (v3.5-0) [https://CRAN.R-project.org/package=survival]. Significance was determined using a log-rank test. For the Cox proportional hazards models, the ‘coxph’ function from the survival package (v3.5-0) [https://CRAN.R-project.org/package=survival] was used to estimate the coefficients of each of the variables. To calculate the combined flow cytometry and TCRseq immune metric, the frequency of CD45RA⁻CD39⁺ Tregs was multiplied by the Large:Small ratio. In the KM and Cox model analyses, the outcome was a future lung cancer diagnosis (yes/no). The final timepoint and corresponding follow-up time for each patient was selected. This reflected a real-life scenario better than averaging immune metrics and follow-up times. Lesion grade was omitted from the Cox models because no low-grade patients had a future lung cancer diagnosis and so the model was unable to converge, therefore becoming inaccurate.

Paragraphs Relating to Certain Aspects of this Disclosure:
1. A method for determining whether a subject is at risk for having a progressing or high-grade pre-invasive lesion, nodule or small mass, or having a solid malignant tumour, the method comprising:
determining a ratio of activated and/or exhausted T cells:naïve and/or resting T cells in a sample of blood obtained from the subject,
wherein the determining comprises analysing T cells using cytometry to detect the presence or absence of a panel of biomarkers comprising Ki67 and CD39.
2. The method of aspect 1, wherein the subject is at risk if the ratio of activated and/or exhausted T cells:naïve and/or resting T cells is equal to or greater than
a ratio of activated and/or exhausted T cells:naïve and/or resting T cells of a comparison subject, or
an average ratio of activated and/or exhausted T cells:naïve and/or resting T cells of a plurality of comparison subjects.
3. A method for determining whether a subject is at risk for having a progressing or high-grade pre-invasive lesion, nodule or small mass, or having a solid malignant tumour, the method comprising:
determining a ratio of activated and/or exhausted T cells:T cells which are not activated and/or exhausted T cells in a sample of blood obtained from the subject,
wherein the determining comprises analysing T cells using cytometry to detect the presence or absence of a panel of biomarkers comprising Ki67 and CD39.
4. The method of aspect 3, wherein the subject is at risk if the ratio of activated and/or exhausted T cells:T cells which are not activated and/or exhausted is equal to or greater than
a ratio of activated and/or exhausted T cells:T cells which are not activated and/or exhausted of a comparison subject, or
an average ratio of activated and/or exhausted T cells:T cells which are not activated and/or exhausted of a plurality of comparison subjects.
5. The method of aspect 2 or 4 wherein, a comparison subject is selected from:
a) a subject known to have a progressing or high-grade pre-invasive lesion, nodule or small mass or an established solid malignant tumour,
b) the subject at a different time point.
6. The method of aspect 2 or 4 wherein, a plurality of comparison subjects is selected from:
a) a plurality of subjects known to have a progressing or high-grade pre-invasive lesion, nodule or small mass or an established solid malignant tumour,
b) a plurality of healthy subjects, or
c) a plurality of subjects of the general population.
7. The method of any one of the preceding aspects, wherein an activated and/or exhausted T cell expresses CD39 and Ki67 (CD39+ and Ki67+ T cells).
8. The method of any one of the preceding aspects, wherein naïve and/or resting T cells do not express CD39 or Ki67 (CD39− and Ki67− T cells).
9. The method of any one of the preceding aspects, wherein T cells which are not activated and/or exhausted do not express one or both of CD39 or Ki67 (CD39− and Ki67+ T cells, CD39+ and Ki67− T cells, or CD39− and Ki67− T cells).
10. The method of any one of the preceding aspects wherein the panel of biomarkers further comprises one or more biomarkers selected from CD45RA, CCR7, PD-1, CD57 or CD38.
11. The method of any one of the preceding aspects wherein the panel of biomarkers further comprises one or more biomarkers selected from CD3, CD4 or CD8.
12. The method of any one of the preceding aspects wherein the analysis comprises use of a viability dye.
13. The method of any one of aspects 1 to 10, wherein the panel of biomarkers further comprises CD45RA, or CCR7, or CD45RA and CCR7.
14. The method of any one of aspects 1 to 10 wherein, the panel of biomarkers further comprises CD45RA, CCR7, and PD-1.
15. The method of any one of aspects 1 to 10, wherein the panel of biomarkers further comprises CD45RA, CCR7, PD-1 and CD57.
16. The method of any one of aspects 1 to 10, wherein the panel of biomarkers further comprises CD45RA, CCR7, PD-1, CD57 and CD38.
17. The method of any one of aspects 1 to 10, wherein the panel of biomarkers further comprises CD45RA, CCR7, CD57, and CD38.
18. The method of any one of aspects 1 to 10, wherein the panel of biomarkers further comprises CD45RA, PD-1 and CD57.
19. The method of any one of the preceding aspects, wherein the cytometry comprises one or more of flow cytometry, spectral cytometry or mass cytometry, optionally the cytometry comprises flow cytometry.
20. A kit comprising a set of lyophilised antibodies or a fragment thereof, comprising antibodies binding to CD39 and Ki67.
21. The kit according to aspect 20, wherein the set of lyophilised antibodies or fragment thereof comprises antibodies binding to the further biomarkers as defined in any one of aspects 10 to 18.
22. Use of a kit according to aspect 20 or 21 in a method for determining whether a subject is at risk for having a progressing or high-grade pre-invasive lesion, nodule or small mass, or having a solid malignant tumour.
23. A device comprising:
(i) means for receiving a sample of blood, and
(ii) a set of lyophilised antibodies or a fragment thereof, comprising antibodies binding to CD39 and Ki67.
24. A method of treating a subject determined to be at risk for having a progressing or high-grade pre-invasive lesion, nodule or small mass, or having a solid malignant tumour, wherein the method comprises:
   determining whether a subject is at risk for having a progressing or high-grade pre-invasive lesion, nodule or small mass, or having a solid malignant tumour, the method comprising:
determining a ratio of activated and/or exhausted T cells:naïve and/or resting T cells in a sample of blood obtained from the subject,
wherein the determining comprises analysing T cells using cytometry to detect the presence or absence of a panel of biomarkers comprising Ki67 and CD39, and wherein the subject is at risk if
the ratio of activated and/or exhausted T cells:naïve and/or resting T cells is equal to or greater than
a ratio of activated and/or exhausted T cells:naïve and/or resting T cells of a comparison subject, or
an average ratio of activated and/or exhausted T cells:naïve and/or resting T cells of a plurality of comparison subjects, and
providing treatment to said subject, if said subject is determined to be at risk.
25. A method of treating a subject determined to be at risk for having a progressing or high-grade pre-invasive lesion, nodule or small mass, or having a solid malignant tumour, wherein the method comprises:
   determining whether a subject is at risk for having a progressing or high-grade pre-invasive lesion, nodule or small mass, or having a solid malignant tumour, the method comprising:
determining a ratio of activated and/or exhausted T cells:T cells which are not activated and/or exhausted T cells in a sample of blood obtained from the subject, wherein the determining comprises analysing T cells using cytometry to detect the presence or absence of a panel of biomarkers comprising Ki67 and CD39, and wherein the subject is at risk if
the ratio of activated and/or exhausted T cells:T cells which are not activated and/or exhausted is equal to or greater than
a ratio of activated and/or exhausted T cells:T cells which are not activated and/or exhausted of a comparison subject, or
an average ratio of activated and/or exhausted T cells:T cells which are not activated and/or exhausted of a plurality of comparison subjects, and
providing treatment to said subject, if said subject is determined to be at risk.
26. A method for determining whether a subject is at risk for having a progressing or high-grade pre-invasive lesion, nodule or small mass, or having a solid malignant tumour, the method comprising analysing the proportion of T cells in a sample of blood obtained from the subject which are activated and/or exhausted T cells by analysing a trait of the T cells.
27. A method for determining whether a subject is at risk for having a progressing or high-grade pre-invasive lesion, nodule or small mass, the method comprising analysing the proportion of T cells in a sample of blood obtained from the subject which are activated and/or exhausted T cells by analysing a trait of the T cells.
28. The method of paragraph 27 or 28, wherein analysing the proportion of T cells in the sample of blood obtained from the subject which are activated and/or exhausted comprises analysing a plurality of traits of the T cells.
29. The method of paragraph 28, wherein the method further comprises combining results obtained from analysing the plurality of traits.
30. The method of any one of paragraphs 26-29, wherein the trait is a phenotypic trait.
31. The method of paragraph 30, wherein the phenotypic trait is the diversity of T cell receptors on the T cells in the sample of blood, optionally the diversity of CDR3B on TCRs of the T cells in the sample of blood, further optionally the phenotypic trait is analysed using TCR Seq.
32. The method of paragraph 31, wherein the subject is at risk if:
a) the diversity of the repertoire of TCRs is less than the diversity of the repertoire of TCRs of a comparison subject or a plurality of comparison subjects,
b) D50 diversity score is lower than a D50 diversity score obtained from analysing T cells in a sample of blood obtained from a comparison subject or a plurality of comparison subjects,
c) a clonality score is lower than a clonality score obtained from analysing T cells in a sample of blood obtained from a comparison subject or a plurality of comparison subjects, or
d) Hill number is lower than a Hill number obtained from analysing T cells in a sample of blood obtained from a comparison subject or a plurality of comparison subjects,
optionally wherein the comparison subject is a healthy subject, or the plurality of comparison subjects are healthy subjects.
33. The method of paragraph 30, wherein the phenotypic trait is biomarker expression by the T cells in the sample of blood, optionally using cytometry, further optionally wherein cytometry is used to detect the presence or absence of a panel of biomarkers comprising Ki67 and CD39.
34. The method of paragraph 33, wherein the subject is at risk if:
a) the ratio of activated and/or exhausted T cells:naïve and/or resting T cells is equal to or greater than
a ratio of activated and/or exhausted T cells:naïve and/or resting T cells of a comparison subject, or
an average ratio of activated and/or exhausted T cells:naïve and/or resting T cells of a plurality of comparison subjects, or
b) the ratio of activated and/or exhausted T cells:T cells which are not activated and/or exhausted is equal to or greater than
a ratio of activated and/or exhausted T cells:T cells which are not activated and/or exhausted of a comparison subject, or
an average ratio of activated and/or exhausted T cells:T cells which are not activated and/or exhausted of a plurality of comparison subjects.
35. The method of paragraph 33 wherein, a comparison subject is selected from:
a) a subject known to have a progressing or high-grade pre-invasive lesion, nodule or small mass or an established solid malignant tumour,
b) the subject at a different time point.
36. The method of paragraph 33 wherein, a plurality of comparison subjects is selected from:
a) a plurality of subjects known to have a progressing or high-grade pre-invasive lesion, nodule or small mass or an established solid malignant tumour,
b) a plurality of healthy subjects, or
c) a plurality of subjects of the general population.
37. The method of any one of paragraphs 33-36 wherein the panel of biomarkers further comprises one or more biomarkers selected from CD45RA, CCR7, PD-1, CD57 or CD38.
38. The method of any one of paragraphs 33-37, wherein the panel of biomarkers further comprises one or more biomarkers selected from CD3, CD4 or CD8.
39. The method of the preceding paragraphs 33-37, wherein the analysis comprises use of a viability dye.
40. The method of paragraphs 33-36, wherein the panel of biomarkers further comprises CD45RA, or CCR7, or CD45RA and CCR7.
42. The method of paragraphs 33-36, wherein, the panel of biomarkers further comprises CD45RA, CCR7, and PD-1.
43. The method of any one of paragraphs 33-36, wherein the panel of biomarkers further comprises CD45RA, CCR7, PD-1 and CD57.
44. The method of paragraphs 33-36, wherein the panel of biomarkers further comprises CD45RA, CCR7, PD-1, CD57 and CD38.
45. The method of paragraphs 33-36, wherein the panel of biomarkers further comprises CD45RA, CCR7, CD57, and CD38.
48. The method of paragraphs 33-36, wherein the panel of biomarkers further comprises CD45RA, PD-1 and CD57.
47. The method of any one of paragraphs 33-46, wherein the cytometry comprises one or more of flow cytometry, spectral cytometry or mass cytometry, optionally the cytometry comprises flow cytometry.

1-38. (canceled)

39. A method of treating a subject determined to be at risk for having a progressing or high-grade pre-invasive lesion, nodule or small mass, or having a solid malignant tumour, the method comprising:
analysing a proportion of T cells in a sample of blood obtained from the subject which are activated and/or exhausted T cells by analysing a trait of the T cells, wherein the trait is a phenotypic trait, and wherein the analysing comprises:
determining a diversity or clonality of T cells in the sample of blood obtained from the subject, wherein the subject is at risk if
(i) the diversity of a repertoire of T cell receptors (TCRs) is lower than a diversity of the repertoire of TCRs of a comparison subject or a plurality of comparison subjects,
ii) a D50 diversity score is lower than a D50 diversity score obtained from analysing T cells in a sample of blood obtained from a comparison subject or a plurality of comparison subjects,
iii) a clonality score is higher than a clonality score obtained from analysing T cells in a sample of blood obtained from a comparison subject or a plurality of comparison subjects,
iv) a Hill number is lower than a Hill number obtained from analysing T cells in a sample of blood obtained from a comparison subject or a plurality of comparison subjects,
v) a clonality of a repertoire of TCRs occupied by the top n clones is higher than a clonality of a repertoire of TCRs occupied by the top n clones in a comparison subject or a plurality of comparison subjects, wherein n is between 50 and 1000, or
vi) a proportion of a repertoire of TCRs occupied by small clones is lower than a proportion of a repertoire of TCRs occupied by small clones in a comparison subject or a plurality of comparison subjects,
vii) a proportion of a repertoire of TCRs occupied by large clones is higher than a proportion of a repertoire of TCRs occupied by large clones in a comparison subject or a plurality of comparison subjects, or
viii) a ratio of a repertoire of TCRs occupied by large clones:small clones is higher than a ratio of a repertoire of TCRs occupied by large clones:small clones in a comparison subject or a plurality of comparison subjects;
determining the subject is at risk for having a progressing or high-grade pre-invasive lesion, nodule or small mass, or having a solid malignant tumour; and
treating the subject based on the determination that the subject is at risk.

40. The method of claim 39, wherein the solid malignant tumour is a stage I solid malignant tumour.

41. The method of claim 39, wherein the phenotypic trait is a diversity of CDR3B on TCRs of the T cells in the sample of blood.

42. The method of claim 39, wherein determining the diversity or clonality of T cells in the sample of blood obtained from the subject is via T cell receptor sequencing (TCR-seq).

43. The method of claim 39, wherein the method further comprises analysing a proportion of T cells in the sample of blood obtained from the subject which are activated and/or exhausted T cells using biomarker expression, wherein the subject is at risk if
(i) a ratio of activated and/or exhausted T cells:naïve and/or resting T cells is equal to or greater than a ratio of activated and/or exhausted T cells:naïve and/or resting T cells of a comparison subject, or in a plurality of comparison subjects, or
(ii) a ratio of activated and/or exhausted T cells:T cells which are not activated and/or exhausted is equal to or greater than a ratio of activated and/or exhausted T cells:T cells which are not activated and/or exhausted of a comparison subject, or in a plurality of comparison subjects; or
(iii) a proportion of activated and/or exhausted T cells is higher than the proportion of activated and/or exhausted T cells in a comparison subject or a plurality of comparison subjects.

44. The method of claim 43, wherein analysing the proportion of T cells in the sample of blood obtained from the subject which are activated and/or exhausted T cells using biomarker expression is via cytometry.

45. The method of claim 44, wherein the cytometry is flow cytometry.

46. The method of claim 44, wherein cytometry is used to detect the presence or absence of a panel of biomarkers comprising Ki67 and CD39, and wherein the activated and/or exhausted T cells express Ki67 and CD39.

47. The method of claim 46, wherein the panel of biomarkers further comprises:
a) one or more biomarkers selected from CD45RA, CCR7, PD-1, CD57, or CD38;
b) one or more biomarkers selected from CD3, CD4, or CD8;
c) CD45RA, CCR7, or CD45RA and CCR7;
d) CD45RA, CCR7, and PD-1;
e) CD45RA, CCR7, PD-1, and CD57;
f) CD45RA, CCR7, PD-1, CD57, and CD38;
g) CD45RA, CCR7, CD57, and CD38; or
h) CD45RA, PD-1, and CD57.

48. The method of claim 46, wherein the activated and/or exhausted T cells express biomarkers Ki67 and CD39 and do not express biomarkers CD45RA, CCR7, or PD-1.

49. The method of claim 39, wherein the activated and/or exhausted T cells express FoxP3, wherein the T cells are CD4+ T cells.

50. The method of claim 39, wherein the activated and/or exhausted cells express FoxP3 and CD39.

51. The method of claim 48, wherein the activated and/or exhausted T cells further (i) express the biomarker Ki67, or (ii) do not express the biomarker CD45RA.

52. The method of claim 39, wherein n is between 50 and 100.

53. The method of claim 39, wherein the comparison subject is a healthy subject, or the plurality of comparison subjects are healthy subjects.

54. The method of claim 39, wherein the treating comprises administering an anti-cancer therapeutic.

55. The method of claim 39, wherein the treating comprises electrocautery, argon plasma coagulation (APC), cryotherapy, or photodynamic therapy (PDT).

56. The method of claim 39, wherein the subject determined to be at risk for having a progressing or high-grade pre-invasive lesion, nodule or small mass.
